(19) United States
(12) Patent Application Publication
(10) Pub. No.: US 2025/0346875 A1
(43) Pub. Date: Nov. 13, 2025

(54) CRISPR/Cas9-Mediated Exon-Skipping Approach for USH2A-Associated Usher Syndrome

(71) Applicants: Massachusetts Eye and Ear Infirmary, Boston, MA (US); Editas Medicine, Inc., Cambridge, MA (US)

(72) Inventors: Eric A. Pierce, Boston, MA (US); Carrie Marie Margulies, Newton, MA (US); Nachiket D. Pendse, Quincy, MA (US); Sebastian W. Gloskowski, Malden, MA (US); Qin Liu, Boston, MA (US); Morgan L. Maeder, Cambridge, MA (US)

(21) Appl. No.: 19/204,797

(22) Filed: May 12, 2025

Related U.S. Application Data
(63) Continuation of application No. 17/040,629, filed on Sep. 23, 2020, now Pat. No. 12,331,328.
(60) Provisional application No. 62/647,578, filed on Mar. 23, 2018.
(60) Provisional application No. 62/794,402, filed on Jan. 18, 2019.

(51) Int. Cl. C12N 9/22; A61K 48/00; C12N 15/113; C12N 15/86

(52) U.S. Cl. CPC C12N 9/22; A61K 48/0016; C12N 15/113; C12N 15/86; C12N 2310/20; C12N 2320/33; C12N 2750/14143

(57) ABSTRACT

Compositions for use in treating subjects with USH2A-associated retinal and/or cochlear degeneration that result from mutations in exon 13 of the USH2A gene by deletion of exon 13 from the USH2A gene or transcripts, and methods of use thereof, as well as genetically modified animals and cells.

### CLAIM OF PRIORITY

This application is a continuation of U.S. patent application Ser. No. 17/040,629, filed Sep. 23, 2020, which is a 371 U.S. National Stage entry of PCT/US2019/023934, filed on Mar. 25, 2019, which claims the benefit of U.S. Provisional Patent Application Serial Nos. 62/647,578, filed on Mar. 23, 2018, and 62/794,402, filed on Jan. 18, 2019. The entire contents of the foregoing are hereby incorporated by reference.

### SEQUENCE LISTING

This application contains a Sequence Listing that has been submitted electronically as an XML file named “48229-0251002_SL_ST26.XML.” The XML file, created on May 9, 2025, is 193,429 bytes in size. The material in the XML file is hereby incorporated by reference in its entirety.

### TECHNICAL FIELD

Described herein are compositions for use in treating subjects with USH2A-associated retinal and/or cochlear degeneration that result from mutations in exon 13 of the USH2A gene by deletion of exon 13 from the USH2A gene or transcripts, and methods of use thereof, as well as genetically modified animals and cells.

### BACKGROUND

The USH2A gene encodes the transmembrane protein Usherin. Usherin localizes mainly at the periciliary region of mammalian photoreceptors and at the stereocilia or hair bundle of the inner ear hair cells (see, e.g., Maerker et al., Hum Mol Genet. 2008 Jan. 1; 17(1):71-86; Liu et al., Proc Natl Acad Sci USA. 2007 Mar. 13; 104(11):4413-8). The Usherin protein has a large extracellular domain that is proposed to interact with basement membrane collagen IV and fibronectin via laminin domains (see, e.g., Maerker et al., 2008; Reiners et al., Hum Mol Genet. 2005 Dec. 15; 14(24):3933-43). Usherin also interacts with other proteins of USH1 and USH2 complex to form Usher networks (Human Molecular Genetics, 26, 1157-1172). Mutations in USH2A are the most common cause of both Usher syndrome type II and autosomal recessive retinitis pigmentosa (arRP), accounting for approximately 17% of the recessive RP cases [1, 2]. The impairment of both vision and hearing in Usher syndrome results in a reduced ability of the individual to perceive, communicate, and extract vital information from the environment [3]. Longitudinal regression analysis has showed that the disease course for patients with USH2A mutations can be rapidly progressive, particularly with respect to losing visual field and mobility [4].

### SUMMARY

The c.2299delG mutation in exonl3 of the USH2A gene is a single basepair deletion that results in a frameshift and premature stop codon, truncating the protein at exon 132 and truncates protein causing ciliary defects. Exon 13 encodes amino acids 723-936, which span 4 of 8 Laminin EGF-like domains in the protein. As not all of these domains appear to be necessary for proper protein function, complete removal of exon 13 can be used to correct the disease phenotype by restoring the proper reading frame of the gene. Exons 12 and 14 are in frame with each other so deletion of exon 13 by a dual-cut approach, in which one gRNA directs a double-strand break to intron 12 and a second gRNA directs a double-strand break to intron 13, is hypothesized to lead to direct splicing of exon 12 to exon 14, thus generating an in-frame coding sequence lacking several of the Laminin EGF-like domains.

Alternatively, disrupting the exon 13 splice acceptor site using a single gRNA, would provide similar results. As the protein lacking exon 13 retains functionality, this approach could also be applied to other exon 13 mutations, e.g., as known in the art, e.g., as shown in Table A.

Provided herein are nucleic acids comprising sequences encoding a Cas9 protein, and a first gRNA, and a second gRNA, wherein the first and second gRNAs are targeted to sequences flanking exon 13 of an usherin (USH2A) gene of the subject, preferably wherein the target sequence of the first gRNA is in the 3′ 1500 base pairs (bp) of intron 12, and the target sequence of the second gRNA is in the 5′ 1500 bp of intron 13 of the USH2A gene. In some embodiments, the first gRNA comprises a target sequence shown in Table 1 or 6A, and/or wherein the second gRNA comprises a target sequence shown in Table 2 or 6B. In some embodiments, the gRNAs comprise In12_307 with In13_318; In12_307 with Inl3_322; In12_307 with In13_323; In12_307 with In13_327; In12_307 with In13_328; In12_321 with In13_318; In12_321 with Inl3_322; In12_321 with Inl3_323; In12_321 with In13_327; or In12_321 with In13_328.

Also provided herein are nucleic acids comprising sequences encoding a Cas9 protein, and a gRNA targeted to a splice acceptor site for exon 13 of an USH2A gene of the subject. In humans, the splice acceptor site is TAGG where TAG is in intron 12 and G is in exon 13. In some embodiments, the gRNA comprises a target sequence shown in Table 3.

In some embodiments, the nucleic acid encodes S. aureus Cas9, preferably wherein the nucleic acid comprises a Cas9 coding sequence according to SEQ ID NO: 10 or encodes a Cas9 comprising the sequence of SEQ ID NO: 11 of WO 2018/026976.

In some embodiments, the sequences encoding Cas9 comprises a nuclear localization signal, e.g., a C-terminal nuclear localization signal and/or an N-terminal nuclear localization signal; and/or wherein the sequences encoding Cas9 comprises a polyadenylation signal.

In some embodiments, the gRNA is a unimolecular S. aureus gRNA comprising SEQ ID NO:7 or SEQ ID NO: 8 of WO 2018/026976, or the corresponding two-part modular S. aureus gRNA, wherein the crRNA component comprises the underlined section and the tracrRNA component comprises the double underlined section of SEQ ID NO:7 or SEQ ID NO:8 of WO 2018/026976.

In some embodiments, the nucleic acid comprises a viral delivery vector. In some embodiments, the viral delivery vector comprises a promoter for Cas9, preferably a CMV, EFS, or hGRK1 promoter. In some embodiments, the viral delivery vector comprises an adeno-associated virus (AAV) vector.

In some embodiments, the nucleic acid comprises: (i) a first guide RNA comprising a targeting domain sequence selected from the group listed in Table 1 or 6a and a second guide RNA comprising a targeting domain sequence selected from the group listed in Table 2 or 6b, or a single guide RNA comprising a targeting domain sequence selected from the group listed in Table 3; (ii) a first and a second inverted terminal repeat sequence (ITR); and (iii) a promoter for driving expression of the Cas9 selected from the group consisting of a CMV, an EFS, or an hGRK1 promoter. In some embodiments, the gRNAs comprise In12_307 with In13_318; In12_307 with Inl3_322; In12_307 with Inl3_323; In12_307 with Inl3_327; In12_307 with Inl3_328; In12_321 with Inl3_318; In12_321 with Inl3_322; In12_321 with Inl3_323; In12_321 with Inl3_327; or In12_321 with Inl3_328.

Also provided are the nucleic acids described herein for use in therapy, for use in preparation of a medicament; and/or for use in a method of treating a subject who has a condition associated with a mutation in exon 13 of USH2A gene.

In some embodiments, the condition is Usher Syndrome type 2 or autosomal recessive retinitis pigmentosa (arRP). In some embodiments, the AAV vector is delivered to a retina of a subject by injection, such as by subretinal injection, or is delivered to the inner ear of a subject by injection, e.g., through the round window.

Also provided herein are transgenic non-human mammal, e.g., a mouse, wherein the genome of the mouse comprises a mouse USH2A gene lacking exon 12 or a mutation in a splice acceptor site for exon 12 of the USH2A gene, and wherein the cells of the mouse express an usherin protein lacking exon 12; and/or wherein the genome of the mouse comprises a human USH2A gene lacking exon 13 or a mutation in a splice acceptor site for exon 13 of the USH2A gene, and wherein the cells of the mouse express an usherin protein lacking exon 13.

Also provided herein are cells, tissue, or organ (e.g., an eye or cochlea) obtained from the transgenic non-human mammals described herein.

Also provided herein are isolated cells, wherein the genome of the cells comprises a human USH2A gene lacking exon 13 or a mutation in a splice acceptor site for exon 13 of the USH2A gene, and wherein the cells express a human usherin protein lacking exon 13, and wherein the cells do not express a functional mouse usherin protein.

In some embodiments, the cell is a cultured mouse cochlear cell. In some embodiments, the cultured mouse cochlear cell is an Oc-K1 cell.

In addition, provided herein is an isolated human usherin protein lacking exon 13, e.g., comprising SEQ ID NO:2, and a nucleic acid encoding the isolated human usherin protein.

In some embodiments, a CRISPR-Cas9 method of altering a cell described herein comprises forming a first double strand break within intron 12 of the human USH2A gene and a forming a second double strand within intron 13 of the human USH2A gene. In various embodiments described herein, the first double strand break is generated using a gRNA targeting domain sequence selected from Table 1 and the second double strand break is generated using a gRNA targeting domain sequence selected from Table 2.

In some embodiments described herein, a CRISPR-Cas9 method of altering a cell is described, which method comprises the step of forming a first double strand break between nucleotides 216,232,137 to 216,246,584 of chromosome 1 and the step of forming a second double strand break between nucleotides 216,247,227 and 216,250,902 of chromosome 1, wherein the first and second double strand breaks are repaired by NHEJ in a manner that results in the removal of exon 13 of the USH2A gene on chromosome 1. In some embodiments, the step of forming the first strand break comprises contacting the cell with a gRNA which comprises a targeting domain sequence selected from Table 1 and the step of forming the second strand break comprises contacting the cell with a gRNA which comprises a targeting domain sequence selected from Table 2. In various embodiments, a gRNA is configured to form a complex with a Cas9 molecule.

In further embodiments, a CRISPR-Cas9 method of altering a cell is described, which method comprises the step of forming a first double strand break between nucleotides 216,248,383 to 216,248,639 of chromosome 1 and the step of forming a second double strand break between nucleotides 216,245,292 and 216,246,542 of chromosome 1, wherein the first and second double strand breaks are repaired by NHEJ in a manner that results in the removal of exon 13 of the USH2A gene on chromosome 1. In some embodiments, the step of forming the first strand break comprises contacting the cell with a gRNA selected from Table 6a and the step of forming the second strand break comprises contacting the cell with a gRNA selected from Table 6b. In various embodiments, the gRNAs selected from Tables 6a and 6b are configured to form a first and second complex with a Cas9 molecule, respectively.

In various embodiments described herein, the cell is from a subject suffering from Usher syndrome type 2A. In some embodiments, the cell is a retinal cell or a photoreceptor cell. In some embodiments, the photoreceptor cell is a cone photoreceptor cell or a cone cell, a rod photoreceptor cell or a rod cell or a macular cone photoreceptor cell.

In some embodiments, a method of altering a cell comprises contacting the cell with a recombinant viral particle comprising:
(a) a nucleotide sequence encoding a first gRNA molecule comprising a targeting domain sequence selected from Table 1:
(b) a nucleotide sequence encoding a second gRNA molecule comprising a targeting domain sequence selected from Table 2; and
(c) a nucleotide sequence encoding a Cas9 molecule;
wherein said viral particle is capable of delivery to a non-dividing cell, and wherein said contacting results in removal of exon 13 of human USH2A gene.

In some embodiments, the viral particle is an AAV viral particle.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1D: Expression of Ush2a in OC-k1 cells. 1A. RT-PCR assay shows the expression of Ush2a and other cilia proteins, including Whrn, Vlgrl, Sept7, Sept2 and Ehdl, in the OC-k1 cells. 1B. Three experimental replicates of RT-PCR for Ush2a gene in OC-k1 cells. 1C. Ush2a protein detected by anti-Ush2a antibody in OC-1 cells in green. Primary cilia in the OC-k1 cells were labeled with acetylated alpha tubulin in red. 1D. Merged images of 1C, showing that Ush2a protein is localized at the base of primary cilia.

FIGS. 2A-2D: Generation of null Ush2a cell model in OC-k1 cells. 2A.

Schematic illustration of sgRNA (SEQ ID NO:3) for targeting the exon 5 of Ush2a gene on mouse chromosome 1 (SEQ ID NO: 189). 2B. The percentage of in-frame (15%) and out-of-frame (85%) indels introduced by NHEJ in cells transfected with U6-sgRNA and CAG-SpCas9-P2A-GFP plasmids. 2C. Examples of T7E1 assay for individual cell clones after transfection with Cas9/sgRNA. 2D. Allele sequences at the target region of Ush2a gene in clone 4, 17 and J (SEQ ID NOs:4-11).

FIGS. 3A-3D: Ciliogenesis in wild-type and Ush2a null OC-K1 cell lines.

Immunostaining for ciliary markers for clone 4 (3A), 17 (3B) and J (3C). Ac-Tub appears in red stains the ciliary axoneme, ush2a appears in green that co-localizes at the base of cilia. The length of cilia in clone 17 and clone J is significantly shorter (3D).

FIGS. 4A-E: Expression of USH2A-AEx13 cDNA rescues the ciliogenesis in Ush2a null cells. Ush2a null cells from clone J transfected with mouse full-length Ush2a cDNA (4B), human wild-type full length USH2A cDNA (4C) and human USH2A-AEx13 cDNAs (4D). Non-transfected cell used as control (4A). Cilia were labeled with Ac-tubulin and the expression of Ush2a is detected by C-term anti-Ush2a antibody. 4E. The cilia length was significantly increased in cells transfected with wild-type and human USH2A-AEx13 cDNAs as compared to the knockout.

FIGS. 5A-D: Generation of Ush2a-AEx12 mouse lines using CRISPR/Cas9. 5A. Schematic illustration of approach for knocking out exon 12 in mouse Ush2a gene. 5B. sgRNA sequences to target intron 11 (11A (SEQ ID NO:12), 11B (SEQ ID NO:13) and 11C (SEQ ID NO:14)) and intron 12 (12A (SEQ ID NO:15), 12C (SEQ ID NO:16) and 12D (SEQ ID NO:17)). 5C. Cleavage efficiencies of selected sgRNAs on a DNA template derived from Ush2a genomic region surrounding exon 12. 5D. Schematic illustration of deletion of exonl2 and surrounding intronic sequence in 9 Ush2a-AEx12 found mice.

FIGS. 5E-K: Phenotypic rescue by Ush2a-AEx12. 5E, both the wild type and exon 12-skippped Ush2a proteins were localized at the transition zone of photoreceptor sensory cilia in ΔEx12/ΔEx12, ΔEx12/ko, and wt mice. 5F Cochleas isolated from P3 ΔEx12 ΔEx12 and wt mice and stained for phalloidin (top panel), and Ush2a, FM1-43, and phalloidin. 5G, Inner and outer hair cell structures in ΔEx12 ΔEx12, ΔEx12 ko, and wt mice on staining with Ush2a, as compared with the knockout. 5H, Auditory brain stem recordings showing restoration of the ABR response in the Ush2a ko/ko mice by the Ush2a-Exl2 allele as compared to the knockout. 5H, Phalloidin staining showed disrupted bundles in the ko/ko mice, but not in ΔEx12 ΔEx12, ΔEx12 ko, or wt mice. 5J, Arrows indicate the accumulation of GFAP protein in ko retina but not in ΔEx12 ΔEx12, ΔEx12 ko, or wt retina. 5K, Normal cone opsin localization in ΔEx12 ΔEx12 and ΔEx12 ko mice, as compared to ko/ko mice.

FIG. 6. Schematic illustration of two approaches to USH2A gene editing.

Exons 12 and 14 are in frame with each other, so the goal of both approaches is to generate USH2A mRNA without exon 13 and thereby restore reading frame. The skipping of exon 13 results in the formation of a novel domain composed of the n-terminus of Laminin EGF-like (LE) domain repeat 4 and the C-terminus of LE repeat 8.

FIG. 7. The splice acceptor sequence is well-conserved across humans (SEQ ID NO:18), African Green monkeys (SEQ ID NO:19), and cynomolgus monkeys (SEQ ID NO:20). The target site of SaCas9-KKH sgRNAs for disrupting the splicing acceptor of exon 13 in human USH2A gene.

FIG. 8 schematically depicts a gRNA used in certain embodiments of the disclosure (SEQ ID NO:21).

FIG. 9. Schematic illustration of an exemplary AAV vector to deliver SaCas9 and gRNAs. U6, human U6 promoter; hGRK1, human photoreceptor-specific rhodopsin kinase promoter; SV40 SD/SA, SV40 intron sequence; NLS, nuclear localization signal; pA, polyadenylation signal.

FIGS. 10A-B. Screening of top dual gRNA pairs and single splice acceptor gRNA. 10A: Editing of top 16 gRNA pairs for complete removal of USH2A exon 13 as determined by a ddPCR assay. 10B: Total editing of identified single gRNA that fall near the USH2A exon 13 splice acceptor.

FIGS. 11A-D. Editing of USH2A human cells and measurement of USH2A delta exon 13 transcripts. 11A: Total editing of CRL-5923 cells with single gRNA targeting the exon 13 splice acceptor. 11B: Delta 13 USH2A transcripts compared to WT USH2A after editing with single gRNA as shown in 10A. 11C: Percent deletion of exon 13 after editing of CRL-5923 cells with the dual gRNA approach as determined by ddPCR. 11D. Delta 13 USH2A transcripts as a fraction of WT USH2A transcripts after editing with dual gRNAs as shown in 11C.

FIG. 12. Percent expression of delta 13 USH2A transcripts in human retinal explants after editing with dual guide approach delivered as an AAV5.

### DETAILED DESCRIPTION

Despite the success of clinical and pre-clinical studies of AAV mediated gene augmentation therapy for multiple genetic types of inherited retinal degeneration [5-13], developing gene therapy for the USH2A form of arRP has been challenging, because the large size of the USH2A coding sequence (CDS15602 bp, 5202aa) far exceeds the packaging capacity of commonly used AAV viral delivery vectors. The present methods overcome these translational barriers by using a Cas9 gene editing approach for USH2A associated arRP [14, 15]. The CRISPR/Cas system is capable of maintaining the edited gene under its endogenous regulatory elements by directly altering the genomic DNA, thereby avoiding ectopic expression and abnormal gene production that may occur with conventional viral-mediated gene augmentation therapies [14, 15].

The usherin protein encoded by USH2A (GenBank Acc No. NC_000001.11, Reference GRCh38.p7 Primary Assembly, Range 215622894-216423396, complement; SEQ ID NO:1) is a transmembrance protein anchored in the photoreceptor plasma membrane (van Wijk, E., et al., Am J Hum Genet, 2004. 74(4): p. 738-44; Grati, M., et al., J Neurosci, 2012. 32(41): p. 14288-93). Its extracellular portion, which accounts for over 96% of the length of the protein and projects into the periciliary matrix, is thought to have an important structural and a possible signaling role for the long-term maintenance of photoreceptors (van Wijk, E., et al., Am J Hum Genet, 2004. 74(4): p. 738-44; Grati, M., et al., J Neurosci, 2012. 32(41): p. 14288-93). Two isoforms of USH2A have been described. Isoform b (GenBank Acc. No. NM_206933.2 (transcript) and NP_996816.2 (protein)) is most abundantly expressed in retina and is used as the canonical, standard sequence in the literature and in this application. Usherin is a protein with a high degree of homologous domain structures (Liu, X., et al., Proc Natl Acad Sci USA, 2007. 104(11): p. 4413-8). Intracellularly, a PDZ domain has been identified to bind whirlin, whereas extracellularly, several domains are present and in most cases in a repetitive fashion, includingl0 Laminin EGF-like (LE) domains and 35 Fibronectin type 3 (FN3) domains. These repetitive domains comprise over 78% of the protein structure combined. The most common mutation c.2299delG, p.Glu767fs in USH2A gene, which causes approximately 15%-30% of USH2A cases is USA [19, 20], is located in exon 13 that encodes LE domain 5 (aa 747-794) (Liu, X., et al., Proc Natl Acad Sci USA, 2007. 104(11): p. 4413-8). Given the high degree of repetitive regions in usherin, it was hypothesized that an usherin protein that lacks one or more of the repetitive domains would retain partial or complete structural integrity and function, such that the abbreviated USH2A can serve as a therapeutic strategy for Usher syndrome type II and autosomal recessive retinitis pigmentosa (arRP) by skipping the mutant exon in USH2A gene.

As shown herein, Ush2a lacking exon 12 and with exons 11 and 13 fused in frame is expressed and localized correctly in the mouse retina and cochlea. When the Ush2a-ΔEx12 allele was expressed on an Ush2a null background, the Ush2a-ΔEx12 protein appeared to rescue the impaired hair cell structure and auditory function as shown by ABR, as compared to Ush2a^{-/-} mice and also showed early signs of at least partial rescue of retinal phenotype. Without wishing to be bound by theory, this data supports the use of the present compositions and methods to restore sight and/or hearing, e.g., at least partially restore sight and/or hearing, in a subject who has Usher syndrome, e.g., associated with a mutation in exon 13 of USH2A gene. Thus a CRISPR/Cas9-based exon-skipping gene editing strategy to restore the reading frame of USH2A by deleting exon 13 holds therapeutic potential for the treatment of USH2A patients.

In one embodiment, an Ush2A nucleic acid molecule includes a nucleotide sequence that is at least about 85% or more identical to the entire length of SEQ ID NO:1. In some embodiments, the nucleotide sequence is at least about 90%, 91%, 92%, 93%, 94%, 95%, 96%, 97%, 98%, 99% or 100% identical to SEQ ID NO:1.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared.

When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. In another embodiment, the percent identity of two amino acid sequences can be assessed as a function of the conservation of amino acid residues within the same family of amino acids (e.g., positive charge, negative charge, polar and uncharged, hydrophobic) at corresponding positions in both amino acid sequences (e.g., the presence of an alanine residue in place of a valine residue at a specific position in both sequences shows a high level of conservation, but the presence of an arginine residue in place of an aspartate residue at a specific position in both sequences shows a low level of conservation).

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Methods of Treatment CRISPR/Cas-based exon-skipping has been successfully used for restoring the expression of functional dystrophin and dystrophic muscle function in the Duchene muscular dystrophy mouse model [21-25]. The methods described herein include methods for the treatment of disorders associated with mutations in exon 13 of the USH2A gene. Exemplary mutations, including 12 nonsense or frameshift mutations and 7 missense mutations on exon 13 (LOVD database), as shown in Table A, such as the most common missense mutation c.2276G>T.

TABLE A
Reported mutations on exon 13 of USH2A gene

| DNA change | Times | Protein change | Nature | Protein domain | References |
|---|---|---|---|---|---|
| c.2209C > T | 19 | p.(Arg737*) | Stop | Laminin EGF- | Auslender et |
| | | | | like 4 (694-746) | al., 2008 |
| c.2242C > T | 2 | p.(Gln748*) | Stop | Laminin EGF- | Pennings et |
| | | | | like 5 (747-794) | al., 2004 |
| c.2276G > T | 173 | p.(Cys759Phe) | Missense | Laminin EGF- | Dreyer et |
| | | | | like 5 (747-794) | al., 2000 |
| c.2299delG | 597 | p.(Glu767Serfs*21) | frameshift | Laminin EGF- | Dreyer et |
| | | | | like 5 (747-794) | al., 2001 |
| c.2310delA | 1 | p.(Glu771Lysfs*17) | frameshift | Laminin EGF- | Boo et |
| | | | | like 5 (747-794) | al., 2013 |
| c.2391_2392delTG | 2 | p.(Cys797*) | Stop | Laminin EGF- | Baux et |
| | | | | like 6 (795-846) | al., 2014 |
| c.2431A > T | 1 | p.(Lys811*) | Stop | Laminin EGF- | Bonnet et |
| | | | | like 6 (795-846) | al., 2016 |
| c.2431_2432delAA | 5 | p.(Lys811Aspfs*ll) | frameshift | Laminin EGF- | Jaijo et |
| | | | | like 6 (795-846) | al., 2010 |
| c.2440C > T | 2 | p.(Gln814*) | Stop | Laminin EGF- | Vona et |
| | | | | like 6 (795-846) | al., 2014 |
| c.2525dup | 1 | p.(Leu843Profs*8) | frameshift | Laminin EGF- | Dreyer et |
| | | | | like 6 (795-846) | al., 2008 |
| c.2541C > A | 1 | p.(Cys847*) | Stop | Laminin EGF- | Weston et |
| | | | | like 7 (847-899) | al., 2000 |
| c.2610C > A | 19 | p.(Cys870*) | Stop | Laminin EGF- | Le Quesne |
| | | | | like 7 (847-899) | Stabej et |
| | | | | | al., 2012 |
| c.2755C > T | 3 | p.(Gln919*) | Stop | Laminin EGF- | Baux et |
| | | | | like 8 (900-950) | al., 2014 |
| c.2761delC | 1 | p.(Leu921Cysfs*46) | frameshift | Laminin EGF- | Seyedahma |
| | | | | like 8 (900-950) | di et al., |
| | | | | | 2004 |
| c.2176T > C | 1 | p.(Cys726Arg) | Missense | Laminin EGF- | Besnard, |
| | | | | like 4 (694-746) | Garcia- |
| | | | | | Garcia et |
| | | | | | al., 2014 |
| c.2236C > G | 1 | p.(Pro746Ala) | Missense | Laminin EGF- | Le Quesne |
| | | | | like 4 (694-746) | Stabej et |
| | | | | | al., 2012 |
| c.2296T > C | 4 | p.(Cys766Arg) | Missense | Laminin EGF- | — |
| | | | | like 5 (747-794) | |
| c.2332G > T | 3 | p.(Asp778Tyr) | Missense | Laminin EGF- | Aller et |
| | | | | like 5 (747-794) | al., 2006 |
| c.2776C > T | 1 | p.(Arg926Cys) | Missense | Laminin EGF- | Zhao et |
| | | | | like 8 (900-950) | al., 2014 |
| c.2802T > G | 15 | p.(Cys934Trp) | Missense | Laminin EGF- | Xu et |
| | | | | like 8 (900-950) | al., 2011 |

In some embodiments, the disorder is Usher syndrome, e.g., type 2 Usher syndrome. Subjects with type 2 Usher syndrome typically have moderate to severe^{PGP}-2¹,T¹,M hearing loss at birth, and vision that becomes progressively impaired starting in adolescence. In some embodiments, the disorder is autosomal recessive retinitis pigmentosa (arRP). Generally, the methods include administering a therapeutically effective amount of a genome editing system as described herein, to a subject who is in need of, or who has been determined to be in need of, such treatment. The term “genome editing system” refers to any system having RNA-guided DNA editing activity. Genome editing systems of the present disclosure include at least two components adapted from naturally occurring CRISPR systems: a gRNA and an RNA-guided nuclease. These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution. See, e.g., WO2018/026976 for a full description of genome editing systems.

As used in this context, to “treat” means to ameliorate at least one symptom of the disorder associated with mutations in exon 13 of the USH2A gene. Often, these mutations result in hearing loss and/or loss of sight; thus, a treatment comprising administration of a therapeutic gene editing system as described herein can result in a reduction in hearing impairment and/or visual impairment; a reduction in the rate of progression of hearing loss and/or vision loss; and/or a return or approach to normal hearing and/or vision. Hearing and vision can be tested using known methods, e.g., electroretinogram, optical coherence tomography, videonystagmography, and audiology testing.

The methods can be used to treat any subject (e.g., a mammalian subject, preferably a human subject) who has a mutation in exon 13 of the USH2A gene, e.g., the c.2299delG mutation or c.2276G>T mutation, e.g., in one or both alleles of USH2A. As used herein, an “allele” is one of a pair or series of genetic variants of a polymorphism (also referred to as a mutation) at a specific genomic location. As used herein, “genotype” refers to the diploid combination of alleles for a given genetic polymorphism. A homozygous subject carries two copies of the same allele and a heterozygous subject carries two different alleles. Methods for identifying subjects with such mutations are known in the art; see, e.g., Yan et al., J Hum Genet. 2009 Dec; 54(12): 732-738; Leroy et al., Exp Eye Res. 2001 May; 72(5):503-9; or Consugar et al., Genet Med. 2015 April; 17(4):253-261. For example, gel electrophoresis, capillary electrophoresis, size exclusion chromatography, sequencing, and/or arrays can be used to detect the presence or absence of the allele or genotype.

Amplification of nucleic acids, where desirable, can be accomplished using methods known in the art, e.g., PCR. In one example, a sample (e.g., a sample comprising genomic DNA), is obtained from a subject. The DNA in the sample is then examined to identify or detect the presence of an allele or genotype as described herein. The allele or genotype can be identified or determined by any method described herein, e.g., by Sanger sequencing or Next Generation Sequencing (NGS). Since the exon 13 is 643 bp in size, thus the genotyping of the patients with exon 13 mutations is simple and straight forward using Sanger sequencing or NGS. Other methods can include hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular mutation (also referred to as a polymorphic variant).

Other methods of nucleic acid analysis can include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP) (Schafer et al., Nat. Biotechnol. 15:33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)); denaturing high performance liquid chromatography (DHPLC, Underhill et al., Genome Res. 7:996-1005 (1997)); infrared matrix-assisted laser desorption/ionization (IR-MALDI) mass spectrometry (WO 99/57318); mobility shift analysis (Orita et al., Proc. Natl. Acad. Sci. USA 86:2766-2770 (1989)); restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, and combinations of such methods. See, e.g., Gerber et al., U.S. Patent Publication No. 2004/0014095 which is incorporated herein by reference in its entirety.

In certain aspects, the present disclosure provides AAV vectors encoding CRISPR/Cas9 genome editing systems, and on the use of such vectors to treat USH2A associated disease. Exemplary AAV vector genomes are schematized in FIG. 9, which illustrates certain fixed and variable elements of these vectors: inverted terminal repeats (ITRs), one or two gRNA sequences and promoter sequences to drive their expression, a Cas9 coding sequence and another promoter to drive its expression (an exemplary construct for use in the Method Two described herein would be the same as that illustrated in FIG. 9, but with only 1 gRNA and U6). Each of these elements is discussed in detail herein. Although FIG. 9 shows a single vector used to deliver a Cas9 and two gRNAs, in some embodiments a plurality of vectors are used, e.g., wherein one vector is used to deliver Cas9, and another vector or vectors is used to deliver one or more gRNAs (e.g., one vector for one gRNA, one vector for two gRNAs, or two vectors for each of two gRNAs).

RNA-guided nucleases/Cas9

Various RNA-guided nucleases can be used in the present methods, e.g., as described in WO 2018/026976. In some embodiments, the RNA-guided nuclease used in the present methods and compositions is a S. aureus Cas9 or a S. pyogenes cas9. In some embodiments of this disclosure a Cas9 sequence is modified to include two nuclear localization sequences (NLSs) (e.g., PKKKRKV (SEQ ID NO:22) at the C- and N-termini of the Cas9 protein, and a mini-polyadenylation signal (or Poly-A sequence). An exemplary NLS is SV40 large T antigen NLS (PKKKRRV (SEQ ID NO:23)) and nucleoplasmin NLS (KRPAATKKAGQAKKKK (SEQ ID NO:24)). Other NLSs are known in the art; see, e.g., Cokol et al., EMBO Rep. 2000 Nov. 15; 1(5):411-415; Freitas and Cunha, Curr Genomics. 2009 December; 10(8): 550-557.An exemplary polyadenylation signal is TAGCAATAAAGGATCGTTTATTTTCATTGGAAGCGTGTG TTGGTTTTTTGATCAGGCGCG (SEQ ID NO:25)). Exemplary S. aureus Cas9 sequences (both nucleotide and peptide) are described in Table 4 of WO 2018/026976, e.g., SEQ ID NOs 10 and 11 therein.

Guide RNAs

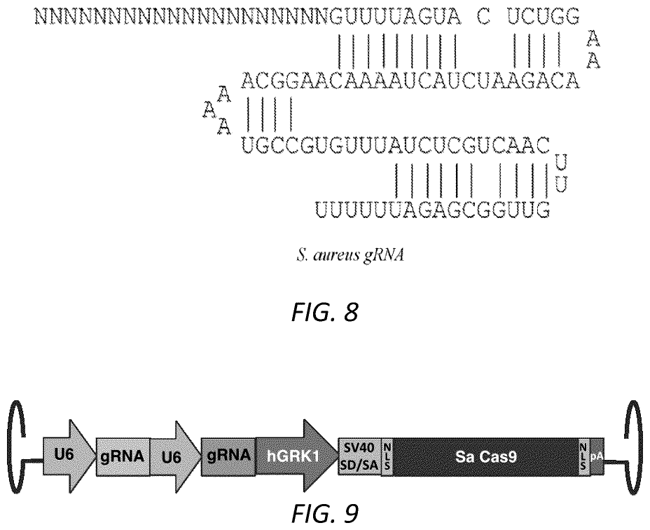

In some embodiments, the gRNAs used in the present disclosure can be unimolecular or modular, as described below. An exemplary unimolecular S. aureus gRNA is shown in FIG. 8, and exemplary DNA and RNA sequences corresponding to unimolecular S. aureus gRNAs are shown below:

DNA:
(SEQ ID NO: 26)
[N_{16-24]}GTTTTAGTACTCTGgaaaCAGAATCTACTAAAACAAGGCAA
AATGCCGTGTTTATCTCGTCAACTTGTTGGCGAGATTTTTT
and
RNA:
(SEQ ID NO: 27)
[N_{16-24]}GUUUUAGUACUCUGgaaaCAGAAUCUACUAAAACAAGGC
AAAAUGCCGUGUUUAUCUCGUCAACUUGUUGGCGAGAUUUUUU.
DNA:
(SEQ ID NO: 28)
[N_{16-24]}GTTATAGTACTCTGgaaaCAGAATCTACTATAACAAGGCAA
AATGCCGTGTTTATCTCGTCAACTTGTTGGCGAGATTTTTT
and
RNA:
(SEQ ID NO: 29)
[N_{16-24]}GUUAUAGUACUCUGgaaaCAGAAUCUACUAUAACAAGG
CAAAAuGCCGUGUUUAUCUCGUCAACUUGUUGGCGAGAUUUUUU.

It should be noted that, while FIG. 8 depicts a targeting domain of 20 nucleotides, the targeting domain can have any suitable length. As indicated by the “N16-24” notation in the sequences above, gRNAs used in the various embodiments of this disclosure preferably include targeting domains of between 16 and 24 (inclusive) bases in length at their 5′ ends, and optionally include a 3′ U6 termination sequence as illustrated.

The gRNA in FIG. 8 is depicted as unimolecular, but in some instances modular guides can be used. In the exemplary unimolecular gRNA sequences above, a 5′ portion corresponding to a crRNA (bold) is connected by a GAAA linker (lower case) to a 3′ portion corresponding to a tracrRNA. Skilled artisans will appreciate that two-part modular gRNAs can be used that correspond to the bold and double underlined sections.

Either one of the gRNAs presented above can be used with any of targeting sequences in tables 1-3, and two gRNAs in a pair do not necessarily include the same backbone sequence. Additionally, skilled artisans will appreciate that the exemplary gRNA designs set forth herein can be modified in a variety of ways, which are described below or are known in the art; the incorporation of such modifications is within the scope of this disclosure.

### Method One: Dual-gRNA Deletion of Exon 13

Described herein are two approaches for treating subjects with mutations in exon 13 of USH2A. The first makes use of dual-gRNAs for deletion of exon 13. Two gRNAs (one in intron 12, one in intron 13) are used in combination to cut out a segment of DNA including exon 13. In addition to deleting this segment, it may also be inverted and reinserted. In the present studies, inversion of the exon was seen as commonly as deletion, and the inverted version was equally functional; without wishing to be bound by theory, the rearrangement may remove the functional splice sites, so the protein still lacks exon 13 and thus corrects the phenotype.

In some embodiments, this approach uses Staphylococcus aureus Cas9 (SaCas9) and corresponding gRNAs. SaCas9 is one of several smaller Cas9 orthologues that are suited for viral delivery (Horvath et al., J Bacteriol 190, 1401-1412 (2008); Ran et al., Nature 520, 186-191 (2015); Zhang et al., Mol Cell 50, 488-503 (2013)). The wild type recognizes a longer NNGRRT PAM that is expected to occur once in every 32 bps of random DNA; or the alternative NNGRRA PAM. Preferably, the 5′ base ofeach gRNAis a G, and the protospacer is length 20, 21 or 22 nucleotides, and the target sequence falls in the 3′ 1500 bp of intron 12 or 5′ 1500 bp of intron 13.

The methods can include using two gRNAs, one that targets intron 12, and one that targets intron 13. The genomic coordinates of introns 12 and 13 are provided in the following table.

| | Genomic | Focused Genomic |
|---|---|---|
| | Coordinates | Coordinates |
| Intron 12 | Chr 1: 216, 232, 137- | Chr 1: 216, 248, 383- |
| | 216, 246, 584 | 216, 248, 639 |
| Intron 13 | Chr 1: 216, 247, 227- | Chr 1: 216, 245, 292- |
| | 216, 250, 902 | 216, 246, 542 |

Tables 1 and 2 provide exemplary sequences for the gRNAs in exons 12 and 13, respectively. Note that in the sequences provided herein, the actual gRNA would have U in place ofT.

TABLE 1
Exemplary Guide RNAs for approach 1: Intron 12

| | | SEQ | | | |
|---|---|---|---|---|---|
| gRNA | | ID | | | hg38 |
| Name | gRNA sequence | NO: | Length | Strand | coordinates |
| in12_67 | GAGAGCATGATTTATATTAA | 30. | 20 | − | chr1:216248081-216248101 |
| in12_68 | GGCCCCCTATGGCATTGCTT | 31. | 20 | − | chr1:216247344-216247364 |
| in12_69 | GTCCTTTTTTATTTTCCTTA | 32. | 20 | − | chr1:216248638-216248658 |
| in12_70 | GTGATGGATACATTAATTAG | 33. | 20 | + | chr1:216247802-216247822 |
| in12_71 | GGATATTTGGAAACTATCTA | 34. | 20 | − | chr1:216248361-216248381 |
| in12_72 | GCATATCATTTAATTTCAAT | 35. | 20 | + | chr1:216248619-216248639 |
| in12_73 | GTAGAAAAGCATTTCCTAAA | 36. | 20 | + | chr1:216248500-216248520 |
| in12_74 | GTCAGCTGATAACTATTTTT | 37. | 20 | + | chr1:216247938-216247958 |
| in12_75 | GTACTTATCATGTTTTTGGG | 38. | 20 | − | chr1:216247777-216247797 |
| in12_76 | GAAAAGCATTTCCTAAACTGG | 39. | 21 | + | chr1:216248503-216248524 |
| in12_77 | GCATATCATTTAATTTCAATA | 40. | 21 | + | chr1:216248619-216248640 |
| in12_78 | GGAGAGCATGATTTATATTAA | 41. | 21 | − | chr1:216248081-216248102 |
| in12_79 | GAACTTATTCATTCTGTCTAG | 42. | 21 | − | chr1:216247663-216247684 |
| in12_80 | GTTATTTAAATTCATGGATAT | 43. | 21 | − | chr1:216248375-216248396 |
| in12_81 | GGCATATCATTTAATTTCAAT | 44. | 21 | + | chr1:216248618-216248639 |
| in12_82 | GCCCCCTATGGCATTGCTTGT | 45. | 21 | − | chr1:216247342-216247363 |
| in12_83 | GGTCCTTTTTTATTTTCCTTA | 46. | 21 | − | chr1:216248638-216248659 |
| in12_84 | GGTGATGGATACATTAATTAG | 47. | 21 | + | chr1:216247801-216247822 |
| in12_85 | GTTATTTAAATTCATGGATATT | 48. | 22 | − | chr1:216248374-216248396 |
| in12_86 | GTTTGTGTCTCGTCTATCTTGA | 49. | 22 | − | chr1:216247274-216247296 |
| in12_87 | GATAAAAGGGTATTACAAGGCA | 50. | 22 | + | chr1:216248288-216248310 |
| in12_88 | GGCCCCCTATGGCATTGCTTGT | 51. | 22 | − | chr1:216247342-216247364 |
| in12_89 | GGCATATCATTTAATTTCAATA | 52. | 22 | + | chr1:216248618-216248640 |
| in12_90 | GAAAAGCATTTCCTAAACTGGA | 53. | 22 | + | chr1:216248503-216248525 |
| in12_307 | GAATTTAAATAACTCACTGC | 54. | 20 | + | chr1:216248383-216248403 |
| in12_308 | GCTTTTCTACATATGAGTTC | 55. | 20 | − | chr1:216248490-216248510 |
| in12_309 | GATGATACGAACACAAAATA | 56. | 20 | + | chr1:216247308-216247328 |
| in12_310 | GGATTAAACCAAAAATTGCC | 57. | 20 | − | chr1:216248029-216248049 |
| in12_311 | GGAGTACACATATACATTTT | 58. | 20 | + | chr1:216248204-216248224 |
| in12_312 | GTTAAAGAACTTGCCTTCAT | 59. | 20 | − | chr1:216248577-216248597 |
| in12_313 | GTGCTCATTTAAAATTATAG | 60. | 20 | + | chr1:216247551-216247571 |
| in12_314 | GCAGTGAGTTATTTAAATTC | 61. | 20 | − | chr1:216248383-216248403 |
| in12_315 | GATGTTTAATAAAAGGTTAAG | 62. | 21 | + | chr1:216247624-216247645 |
| in12_316 | GTGATGGATACATTAATTAGC | 63. | 21 | + | chr1:216247802-216247823 |
| in12_317 | GATAAAATATATTTAAAAGGT | 64. | 21 | + | chr1:216247231-216247252 |
| in12_318 | GTAAGGTTATTCTAAAAGATAA | 65. | 22 | + | chr1:216248271-216248293 |
| in12_319 | GAACTTGCCTTCATTGGAGTTC | 66. | 22 | − | chr1:216248569-216248591 |
| in12_320 | GGTGATGGATACATTAATTAGC | 67. | 22 | + | chr1:216247801-216247823 |
| in12_321 | GAAATTAAATGATATGCCTTAG | 68. | 22 | − | chr1:216248614-216248636 |
| in12_322 | GAAGGCAAGTTCTTTAACAGTG | 69. | 22 | + | chr1:216248579-216248601 |
| in12_323 | GATAAAATAGAGGAGCATACAA | 70. | 22 | − | chr1:216248051-216248073 |

TABLE 2
Exemplary Guide RNAs for approach 1: Intron 13

| | | SEQ | | | |
|---|---|---|---|---|---|
| gRNA | | ID | | | hg38 |
| Name | gRNA sequence | NO: | Length | Strand | coordinates |
| in13_73 | GTCCCCTTCTGAGAGAGAGA | 71. | 20 | + | chr1:216245460-216245480 |
| in13_74 | GGTCTATCCCTCTCCCAATT | 72. | 20 | + | chr1:216246017-216246037 |
| in13_75 | GGGGTCTATCCCTCTCCCAA | 73. | 20 | + | chr1:216246015-216246035 |
| in13_76 | GAAAGATAAGTTTGTATATA | 74. | 20 | + | chr1:216246049-216246069 |
| in13_77 | GCCATATACCCATGTAGAGA | 75. | 20 | + | chr1:216245426-216245446 |
| in13_78 | GAGGTAAAGTCCCCTTCTGA | 76. | 20 | + | chr1:216245452-216245472 |
| in13_79 | GGTGGCCATATACCCATGTA | 77. | 20 | + | chr1:216245422-216245442 |
| in13_80 | GGACCACTGCAGTCAGGACT | 78. | 20 | − | chr1:216245556-216245576 |
| in13_81 | GAAGCCACAAACCAGAAACA | 79. | 20 | + | chr1:216246494-216246514 |
| in13_82 | GAAGTTACCTAAGTTAACAA | 80. | 20 | + | chr1:216246518-216246538 |
| in13_83 | GTAGAAATTGAGTCTCAATT | 81. | 20 | + | chr1:216246324-216246344 |
| in13_84 | GTAATCAGTGTCAACCAGGT | 82. | 20 | − | chr1:216246582-216246602 |
| in13_85 | GTGGTAAGTATCCCAGAAGA | 83. | 20 | − | chr1:216245191-216245211 |
| in13_86 | GAGAGGTAAAGTCCCCTTCT | 84. | 20 | + | chr1:216245450-216245470 |
| in13_87 | GTAGAATTATAAACAATTTC | 85. | 20 | + | chr1:216245391-216245411 |
| in13_88 | GGTAAAGTCCCCTTCTGAGA | 86. | 20 | + | chr1:216245454-216245474 |
| in13_89 | GGGTCTATCCCTCTCCCAAT | 87. | 20 | + | chr1:216246016-216246036 |
| in13_90 | GAGAGAGGTAAAGTCCCCTT | 88. | 20 | + | chr1:216245448-216245468 |
| in13_91 | GTAAAGTCCCCTTCTGAGAGA | 89. | 21 | + | chr1:216245455-216245476 |
| in13_92 | GGGTCTATCCCTCTCCCAATT | 90. | 21 | + | chr1:216246016-216246037 |
| in13_93 | GGGTGGCCATATACCCATGTA | 91. | 21 | + | chr1:216245421-216245442 |
| in13_94 | GCTATATGTTCTAGTTTTATA | 92. | 21 | − | chr1:216246135-216246156 |
| in13_95 | GATATAAATACAGTAATGATT | 93. | 21 | + | chr1:216246455-216246476 |
| in13_96 | GAGACAAATGAATATGTATCA | 94. | 21 | + | chr1:216245514-216245535 |
| in13_97 | GGCCATATACCCATGTAGAGA | 95. | 21 | + | chr1:216245425-216245446 |
| in13_98 | GAGTCAATTCGAATTTTCCTC | 96. | 21 | + | chr1:216245319-216245340 |
| in13_99 | GGTGGTAAGTATCCCAGAAGA | 97. | 21 | − | chr1:216245191-216245212 |
| in13_100 | GTAGAAGCCACAAACCAGAAA | 98. | 21 | + | chr1:216246491-216246512 |
| in13_101 | GGTAGAATTATAAACAATTTC | 99. | 21 | + | chr1:216245390-216245411 |
| in13_102 | GTCAATTTTATTTTCCAGAGA | 100. | 21 | − | chr1:216246251-216246272 |
| in13_103 | GGAAAGATAAGTTTGTATATA | 101. | 21 | + | chr1:216246048-216246069 |
| in13_104 | GGGGTCTATCCCTCTCCCAAT | 102. | 21 | + | chr1:216246015-216246036 |
| in13_105 | GTGTAAGATCAAAGAGACAAG | 103. | 21 | − | chr1:216245696-216245717 |
| in13_106 | GTCCCCTTCTGAGAGAGAGAGA | 104. | 22 | + | chr1:216245460-216245482 |
| in13_107 | GATATAAATACAGTAATGATTT | 105. | 22 | + | chr1:216246455-216246477 |
| in13_365 | GGGGTCTATCCCTCTCCCAATT | 106. | 22 | + | chr1:216246015-216246037 |
| in13_393 | GGCTATATGTTCTAGTTTTATA | 107. | 22 | − | chr1:216246135-216246157 |
| in13_404 | GTGTAGAAATTGAGTCTCAATT | 108. | 22 | + | chr1:216246322-216246344 |
| in13_405 | GAGAGAGGTAAAGTCCCCTTCT | 109. | 22 | + | chr1:216245448-216245470 |
| in13_408 | GGTAAAGTCCCCTTCTGAGAGA | 110. | 22 | + | chr1:216245454-216245476 |
| in13_417 | GCCGATCGGATTTATTTCATAA | 111. | 22 | − | chr1:216246342-216246364 |
| in13_421 | GATTTTTAAAAAACTGTTAAAA | 112. | 22 | + | chr1:216246284-216246306 |
| in13_437 | GCCATATACCCATGTAGAGAAA | 113. | 22 | + | chr1:216245426-216245448 |
| in13_438 | GAGAAGTTACCTAAGTTAACAA | 114. | 22 | + | chr1:216246516-216246538 |
| in13_452 | GTGTAATCAGTGTCAACCAGGT | 115. | 22 | − | chr1:216246582-216246604 |
| in13_454 | GTCAATTTTATTTTCCAGAGAA | 116. | 22 | − | chr1:216246250-216246272 |
| in13_460 | GAGAGGTAAAGTCCCCTTCTGA | 117. | 22 | + | chr1:216245450-216245472 |
| in13_466 | GAGGTAAAGTCCCCTTCTGAGA | 118. | 22 | + | chr1:216245452-216245474 |
| in13_472 | GTATATGGCCACCCTATGTCCC | 119. | 22 | − | chr1:216245413-216245435 |
| in13_478 | GTCTCCATAATCTTCCTGTCTT | 120. | 22 | + | chr1:216245173-216245195 |
| in13_314 | GTTAAATAGTTATATATGTG | 121. | 20 | + | chr1:216246426-216246446 |
| in13_315 | GACCACTGCAGTCAGGACTC | 122. | 20 | − | chr1:216245555-216245575 |
| in13_316 | GTAAGATCAAAGAGACAAGA | 123. | 20 | − | chr1:216245695-216245715 |
| in13_317 | GGGTGTCACGTACTTATAAA | 124. | 20 | − | chr1:216245254-216245274 |
| in13_318 | GAGACAAGAAGGAATTGATG | 125. | 20 | − | chr1:216245684-216245704 |
| in13_319 | GATTTACTTCAAGTGTAGAAA | 126. | 21 | + | chr1:216246310-216246331 |
| in13_320 | GTCCTTCTCCTTAGGTTTTTA | 127. | 21 | + | chr1:216245572-216245593 |
| in13_321 | GCATGGCCCAATTATCCTAGG | 128. | 21 | + | chr1:216245739-216245760 |
| in13_322 | GTGTGATTTGCTTGCCAGAGA | 129. | 21 | − | chr1:216245802-216245823 |
| in13_323 | GAAGTTACCTAAGTTAACAAA | 130. | 21 | + | chr1:216246518-216246539 |
| in13_324 | GGACCACTGCAGTCAGGACTC | 131. | 21 | − | chr1:216245555-216245576 |
| in13_325 | GGGGTGTCACGTACTTATAAA | 132. | 21 | − | chr1:216245254-216245275 |
| in13_326 | GTTAATTGATTGCAAATTTGA | 133. | 21 | + | chr1:216245088-216245109 |
| in13_327 | GAGGTAACCAACCAAACAAAA | 134. | 21 | + | chr1:216245296-216245317 |
| in13_328 | GTGTAAGATCAAAGAGACAAGA | 135. | 22 | − | chr1:216245695-216245717 |
| in13_329 | GGTCCTTCTCCTTAGGTTTTTA | 136. | 22 | + | chr1:216245571-216245593 |
| in13_379 | GTTATAATTTCTAGAGGAAAAT | 137. | 22 | − | chr1:216245331-216245353 |
| in13_384 | GTACGTGACACCCCTGGCCACA | 138. | 22 | + | chr1:216245261-216245283 |
| in13_392 | GTTTTTTAAAAATCAGATCAAC | 139. | 22 | − | chr1:216246276-216246298 |
| in13_463 | GTCCTGTCAGCCAATATTAGCT | 140. | 22 | − | chr1:216245869-216245891 |
| in13_469 | GCCAATATTAGCTCTGAGTTAT | 141. | 22 | − | chr1:216245860-216245882 |
| in13_480 | GATTAACCTGAAGGTAAAAAGA | 142. | 22 | + | chr1:216245112-216245134 |

In some embodiments of these methods, any of the intron 12 gRNAs in Table 1 can be used with any of the intron 13 gRNAs in Table 2, though certain combinations may be more suitable for certain applications. It should be noted, notwithstanding the use of “first” and “second” as nomenclature for gRNAs, that any guide in a pair, in intron 12 or intron 13, can be placed in either one of the gRNA coding sequence positions illustrated in FIG. 9.

In some embodiments, one of the combinations of gRNAs in the following table is used; each row shows a preferred combination (e.g., In12_307 with In12_318).

| Intron 12 gRNA | Intron 13 gRNA |
|---|---|
| In12_307 | In13_318 |
| In12_307 | In13_322 |
| In12_307 | In13_323 |
| In12_307 | In13_327 |
| In12_307 | In13_328 |
| In12_321 | In13_318 |
| In12_321 | In13_322 |
| In12_321 | In13_323 |
| In12_321 | In13_327 |
| In12_321 | In13_328 |

In any of the methods described herein, the engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCpl recognize TTTN PAMs that are found 5′ of the protospacer (Id.). In some embodiments, the Cas12a is, e.g., Acidaminococcus sp. BV3L6 Cpf1 (AsCpf1, UniProt U2UMQ6.1) or Lachnospiraceae bacterium ND2006 (LbCpf1, UniProt AOA182DWE3.1), with corresponding gRNAs.

Method Two: Single gRNA Deletion ofExon 13 Splice Acceptor

The second approach makes use of a single gRNA for destruction of the exon 13 splice acceptor. Non-homologous end joining (NHEJ)-mediated indels destroy the splice acceptor, thus preventing exon 13 from being spliced into mRNA.

Preferably, this method uses Staphylococcus aureus wild type or KKH variant SaCas9 (See Kleinstiver et al., Nat Biotechnol. 2015 December; 33(12): 1293-1298; WO 2016/141224) or Casl2a and corresponding gRNAs. Table 3 provides exemplary target sites in the splice acceptor.

TABLE 3
Guide RNAs for approach 2:

| | | SEQ ID | | |
|---|---|---|---|---|
| gRNA Name | Target Site | NO: | Enzyme | Length |
| SA_1 | TATTTTATCTTTAGGGCTTAGG | 143. | SaCas9-KKH | 22 |
| SA_2 | AAATATATTTTATCTTTAGGGC | 144. | SaCas9-KKH | 22 |
| SA_3 | TAAATATATTTTATCTTTAGGG | 145. | SaCas9-KKH | 22 |
| SA_4 | CCTTTTAAATATATTTTATCTT | 146. | SaCas9-KKH | 22 |
| SA_5 | ACCTTTTAAATATATTTTATCT | 147. | SaCas9-KKH | 22 |
| SA_6 | CACCTTTTAAATATATTTTATC | 148. | SaCas9-KKH | 22 |
| SA_7 | GATCACACCTAAGCCCTAAAGA | 149. | SaCas9-KKH | 22 |
| SA_8 | TGATCACACCTAAGCCCTAAAG | 150. | SaCas9-KKH | 22 |
| SA_9 | ATGATCACACCTAAGCCCTAAA | 151. | SaCas9-KKH | 22 |
| SA_10 | GCAATGATCACACCTAAGCCCT | 152. | SaCas9-KKH | 22 |
| SA_11 | TGCAATGATCACACCTAAGCCC | 153. | SaCas9-KKH | 22 |
| SA_12 | TTGCAATGATCACACCTAAGCC | 154. | SaCas9-KKH | 22 |
| SA_13 | ATTGCAATGATCACACCTAAGC | 155. | SaCas9-KKH | 22 |
| SA_14 | CCTTTTAAATATATTTTATCTT | 156. | SaCas9 | 22 |
| SA_18 | ATTTTATCTTTAGGGCTTAGG | 157. | SaCas9-KKH | 21 |
| SA_19 | AATATATTTTATCTTTAGGGC | 158. | SaCas9-KKH | 21 |
| SA_20 | AAATATATTTTATCTTTAGGG | 159. | SaCas9-KKH | 21 |
| SA_21 | CTTTTAAATATATTTTATCTT | 160. | SaCas9-KKH | 21 |
| SA_22 | CCTTTTAAATATATTTTATCT | 161. | SaCas9-KKH | 21 |
| SA_23 | ACCTTTTAAATATATTTTATC | 162. | SaCas9-KKH | 21 |
| SA_24 | ATCACACCTAAGCCCTAAAGA | 163. | SaCas9-KKH | 21 |
| SA_25 | GATCACACCTAAGCCCTAAAG | 164. | SaCas9-KKH | 21 |
| SA_26 | TGATCACACCTAAGCCCTAAA | 165. | SaCas9-KKH | 21 |
| SA_27 | CAATGATCACACCTAAGCCCT | 166. | SaCas9-KKH | 21 |
| SA_28 | GCAATGATCACACCTAAGCCC | 167. | SaCas9-KKH | 21 |
| SA_29 | TGCAATGATCACACCTAAGCC | 168. | SaCas9-KKH | 21 |
| SA_30 | TTGCAATGATCACACCTAAGC | 169. | SaCas9-KKH | 21 |
| SA_31 | CTTTTAAATATATTTTATCTT | 170. | SaCas9 | 21 |
| SA_32 | TTTTATCTTTAGGGCTTAGG | 171. | SaCas9-KKH | 20 |
| SA_33 | ATATATTTTATCTTTAGGGC | 172. | SaCas9-KKH | 20 |
| SA_34 | AATATATTTTATCTTTAGGG | 173. | SaCas9-KKH | 20 |
| SA_35 | TTTTAAATATATTTTATCTT | 174. | SaCas9-KKH | 20 |
| SA_36 | CTTTTAAATATATTTTATCT | 175. | SaCas9-KKH | 20 |
| SA_37 | CCTTTTAAATATATTTTATC | 176. | SaCas9-KKH | 20 |
| SA_38 | TCACACCTAAGCCCTAAAGA | 177. | SaCas9-KKH | 20 |
| SA_39 | ATCACACCTAAGCCCTAAAG | 178. | SaCas9-KKH | 20 |
| SA_40 | GATCACACCTAAGCCCTAAA | 179. | SaCas9-KKH | 20 |
| SA_41 | AATGATCACACCTAAGCCCT | 180. | SaCas9-KKH | 20 |
| SA_42 | CAATGATCACACCTAAGCCC | 181. | SaCas9-KKH | 20 |
| SA_43 | GCAATGATCACACCTAAGCC | 182. | SaCas9-KKH | 20 |
| SA_44 | TGCAATGATCACACCTAAGC | 183. | SaCas9-KKH | 20 |
| SA_45 | TTTTAAATATATTTTATCTT | 184. | SaCas9 | 20 |
| SA_46 | TTTTATCTTTAGGGCTTAGG | 185. | AsCpf1 RVR | 20 |
| SA_47 | AATATATTTTATCTTTAGGG | 186. | LbCpf1 | 20 |
| SA_48 | AAATTGCAATGATCACACCT | 187. | AsCpf1 | 20 |

### AAV Delivery Systems

The methods include delivery of a CRISPR/Cas9 genome editing system, including a Cas9 nuclease and one or two guide RNAs, to a subject in need thereof. The delivery methods can include, e.g., viral delivery, e.g., preferably using an adeno-associated virus (AAV) vector that comprises sequences encoding the Cas9 and guide RNA(s). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol.158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., Pang et al., Vision Research 2008, 48(3):377-385; Khani et al., Invest Ophthalmol Vis Sci. 2007 September;48(9):3954-61; Allocca et al., J. Virol. 2007 81(20):11372-11380). In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and a Cas9 sequence and guide RNA sequence as described herein. In some embodiments, the AAV capsid polypeptide is an Anc80 polypeptide, e.g., Anc80L27; Anc80L59; Anc80L60; Anc80L62; Anc80L65; Anc80L33; Anc80L36; or Anc80L44. In some embodiments, the AAV incorporates inverted terminal repeats (ITRs) derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

Cas9 expression is driven by a promoter known in the art. In some embodiments, expression is driven by one of three promoters: cytomegalovirus (CMV), elongation factor-1 (EFS), or human g-protein receptor coupled kinase-1 (hGRK1), which is specifically expressed in retinal photoreceptor cells. Nucleotide sequences for each of these promoters are provided in Table 5 of WO 2018/026976. Modifications of these sequences may be possible or desirable in certain applications, and such modifications are within the scope of this disclosure.

Expression of the gRNAs in the AAV vector is driven by a promoter known in the art. In some embodiments, a polymerase III promoter, such as a human U6 promoter. An exemplary U6 promoter sequence is presented below:

(SEQ ID NO: 188)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACC.

In some embodiments, the nucleic acid or AAV vector shares at least 80%, 85%, 90%, 95%, 96%, 97%, 98%, 99% or greater sequence identity with one of the^{PGP}-2⁸,D^{NA}nucleic acids or AAV vectors recited above

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., TWEEN® 20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection, for example, subretinal injection or injection through the round window. The concentration of AAV vector within the composition is selected to ensure, among other things, that a sufficient AAV dose is administered to the retina or inner ear of the subject, taking account of dead volume within the injection apparatus and the relatively limited volume that can be safely administered. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal or cochlear space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of all three types of retinal photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

For delivery to the inner ear, injection to the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane (RWM). The RWM, which is the only non-bony opening into the inner ear, is relatively easily accessible in many animal models and administration of viral vector using this route is well tolerated. Administration through the oval window or across the tympanic membrane can also be used. See, e.g., WO2017100791 and U.S. Pat. No. 7,206,639.

For pre-clinical development purposes, systems, compositions, nucleotides and vectors according to this disclosure can be evaluated ex vivo using a retinal explant system, or in vivo using an animal model such as a mouse, rabbit, pig, nonhuman primate, etc. Retinal explants are optionally maintained on a support matrix, and AAV vectors can be delivered by injection into the space between the photoreceptor layer and the support matrix, to mimic subretinal injection. Tissue for retinal explanation can be obtained from human or animal subjects, for example mouse.

Explants are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

Genetically modified Animals and Cells Lacking Exon 13

Also provided herein are non-human genetically modified animals comprising a mutation in exon 13 (or the equivalent exon, for example, exon 12 in the mouse) in the USH2A gene. Such animals are useful as models of disease, e.g., of Usher syndrome or arRP, for studying the function and/or activity of USH2A protein and for identifying and/or evaluating potential therapeutic compounds for treating conditions associated with mutations in exon 13 of the USH2A gene. As used herein, a “genetically modified animal” is a non-human animal, preferably a mammal, more preferably a rodent such as a rat or mouse, in which one or more of the cells of the animal includes a modified gene. Other examples of genetically modified animals include non-human primates, sheep, dogs, cows, goats, chickens, amphibians, and the like.

The genetically modified animals can have a complete deletion of exon 13, an inversion of exon 13, or a mutation that disrupts the exon 13 splice acceptor site, integrated into or occurring in the genome of the cells of a genetically modified animal (e.g., in one or both alleles of the gene in the genome). In preferred embodiments, the animal has had both endogenous USH2A alleles replaced with a human USH2A gene, or has had part of both endogenous USH2A alleles containing the relevant exon and flanking intronic regions replaced with a human USH2A exon 13 and flanking intronic regions, with a complete deletion of exon 13, an inversion of exon 13, or a mutation that disrupts the exon 13 splice acceptor site.

Methods for making genetically modified animals are known in the art; see, e.g., WO2016049024; WO201604925; WO2017124086; WO2018009562; and U.S. Pat. No. 9,901,080. Such techniques include, without limitation, pronuclear microinjection (See, e.g., U.S. Pat. No. 4,873,191), retrovirus mediated gene transfer into germ lines (Van der Putten et al., Proc. Natl. Acad. Sci. USA, 82:6148-1652 (1985)), gene targeting into embryonic stem cells (Thompson et al., Cell 56:313-321 (1989)), electroporation of embryos (Lo, Mol. Cell. Biol., 3:1803-1814 (1983)), and in vitro transformation of somatic cells, such as cumulus or mammary cells, followed by nuclear transplantation (Wilmut et al., Nature, 385:810-813 (1997); and Wakayama et al., Nature, 394:369-374 (1998)); these methods can be modified to use CRISPR as described herein. For example, fetal fibroblasts can be genetically modified using CRISPR as described herein, and then fused with enucleated oocytes. After activation of the oocytes, the eggs are cultured to the blastocyst stage. See, for example, Cibelli et al., Science, 280:1256-1258 (1998)

A founder animal can be identified based upon the presence of a mutation in exon 13 of USH2A in its genome and/or expression of USH2A mRNA lacking exon 13 in tissues or cells of the animals. A genetically modified founder animal can then be used to breed additional animals carrying the modified gene. Moreover, animals carrying a modified encoding an Ush2A protein lacking exon 13 can further be bred to Ush2a knockout animals.

The invention also includes a population of cells isolated from an animal as described herein, as well as primary or cultured cells, e.g., isolated cells, engineered to include a mutation in exon 13 of human USH2A gene or a deletion of exon 12 in the mouse Ush2a gene. The cells can have a complete deletion of exon13, an inversion of exon 13, or a mutation that disrupts the exon 13 splice acceptor site, integrated into or occurs in the genome of the cells. The cells can be from any mammal, e.g., a human or non-human mammal, or other animal.

Further provided herein are nucleic acids (e.g., isolated nucleic acids) that comprise or encode an USH2A mRNA that lacks exon 13, e.g., that have a complete deletion of exon13, an inversion of exon 13, or a mutation that disrupts the exon 13 splice acceptor site, as well as expression and delivery vectors (including viral and non-viral vectors) comprising the nucleic acids, and usherin proteins lacking exon 13. Preferably the sequences are generated using a human USH2A sequence, but they can also be generated from other mammals, including mouse (mRNA: NM_021408.3; syntenic exon: 12) rat (mRNA: NM_001302219.1; syntenic exon 13); chimpanzee (mRNA: XM_016938662.1; syntenic exon: 12); Cynomolgus macaque (macaca fasicularis, mRNA: XM_005540847.2 or XM_005540848.1) and african green monkey (chlorocebus sabeus, mRNA: XM_007988447.1).

### Examples

The invention is further described in the following examples, which do not limit the scope of the invention described in the claims.

### Example 1. USH2A In vitro Model

This Examiner describes the generation of an USH2A knockout cell line.

1. Characterization of OC-k1 cells.

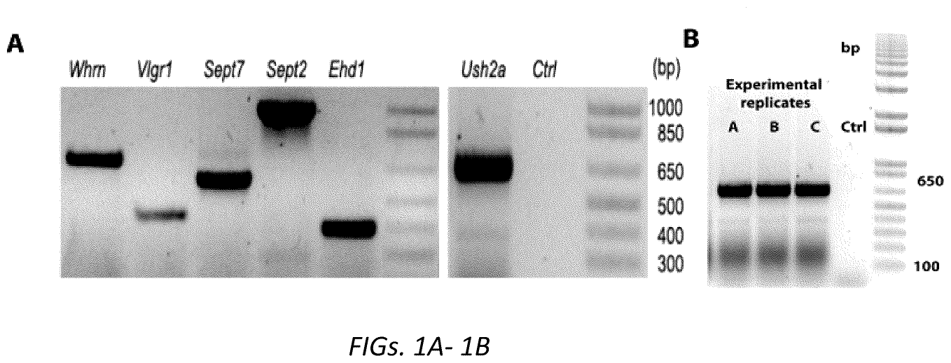

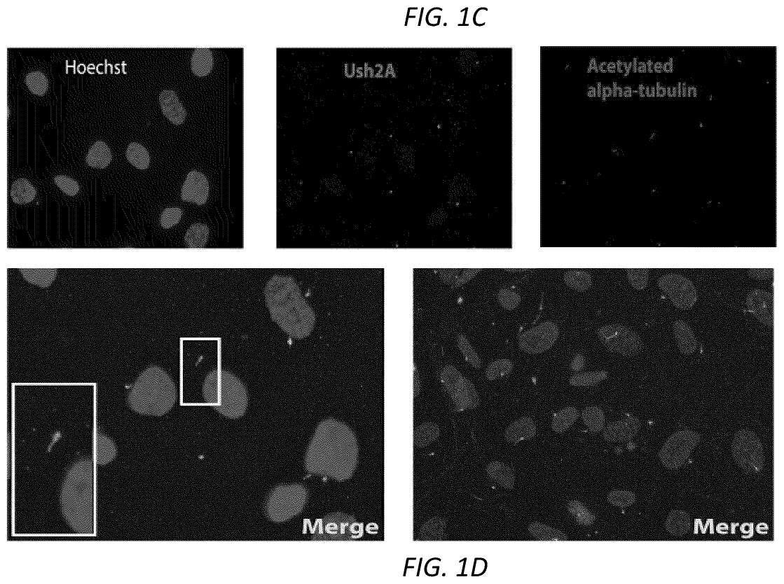

OC-k1 cells were selected as a model systemfor this study. OC-k1 cells are derived from mouse cochlea (Kalinec et al. (1999) Cell biology international 23(3):175-184; Kelley et al. (1993) Development 119(4):1041-1053). These are tri allelic, expressing Ush2a protein and its interacting proteins such as Whrn, Vlgrl in the Usher 2 complex (FIGS. 1A, 1B), making these cells particularly appropriate for these studies. The preliminary results illustrated that the OC-k1 wild type cells stably expressed the Ush2a protein at the base of primary cilia. (FIGS. 1C, 1D).

2. Generation of Ush2A knockout cell line in OC-k1 cells

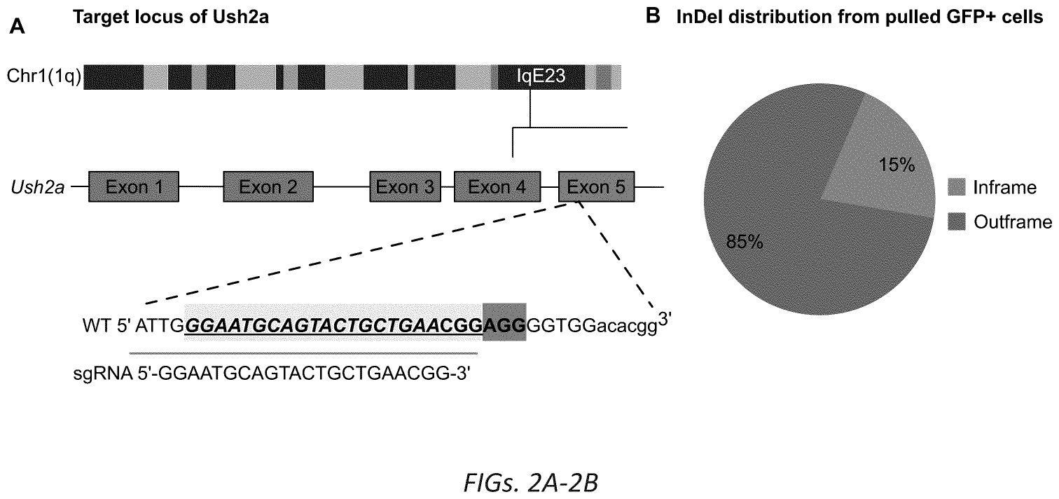

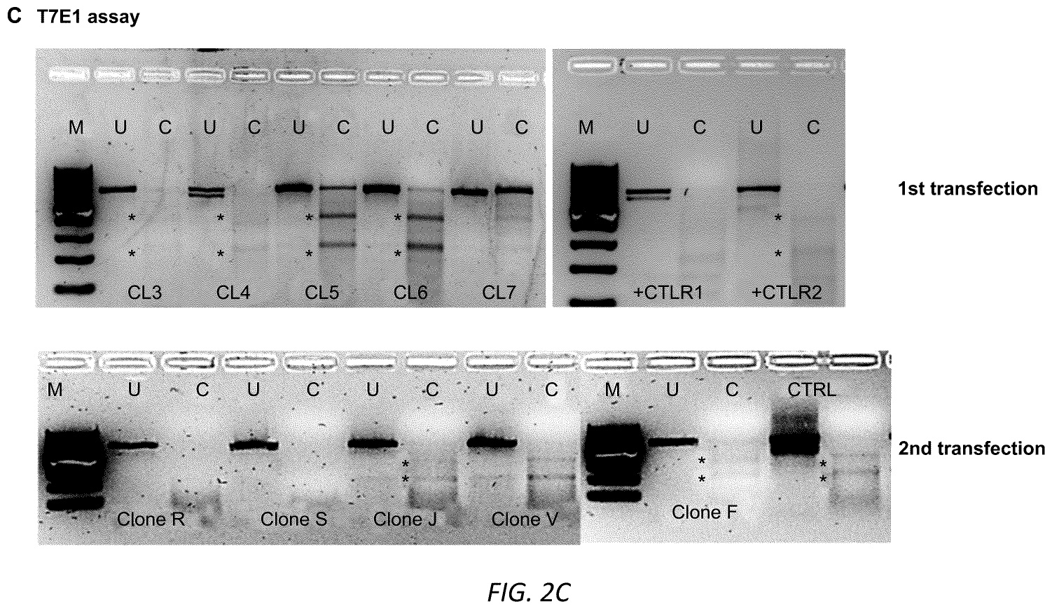

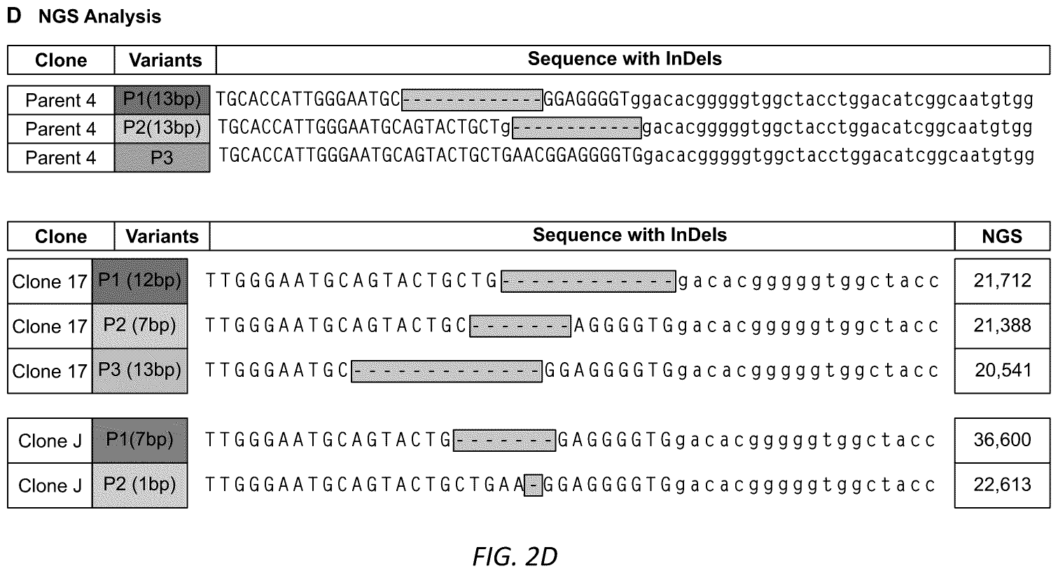

CRISPR/Cas9 technology was used to create the Ush2a null cell model in OC-k1 cells. Guide RNA 5′-GGAATGCAGTACTGCTGAACGG-3′ (SEQ ID NO:3) for wild-type SpCas9 was designed to target the exon 5 of mouse Ush2a gene (FIG. 2A). U6-sgRNA and CAG-SpCas9-P2A-GFP plasmids were CO-transfected into OC-k1 cells using lipofectamine 3000. Two days post-transfection, the GFP positive cells were collected by sorting, plated at low density, and grown to mature colonies. Post genomic DNA isolation from these cells, the region spanning the sgRNA target site was PCR amplified and deep sequencing analysis was performed to get insights into the frequency of NHEJ-induced insertion-deletions (Indels) in Ush2a alleles. A cleavage efficiency of ˜69% was observed with a wide variety of indels with 85% of out-of-frame indels (FIG. 2B). A total of 328 clones were screened with T7E1 assay (FIG. 2C) and found that the clone 4 obtained from first attempt of transfections resulted in a mixed pattern of indels including in-frame, out-of-frame and uncut alleles (FIG. 2D). In order to knockout all three Ush2a alleles in a single clone, clone 4 was re-transfected with the same SpCas9-sgRNA pair and serial dilution of the culture was performed. Of many different single clones analyzed with both T7E1 and NGS analysis, clone J was confirmed to be completely null for Ush2a (two alleles with 7 bp and remaining one allele with lbp out of frame deletions, based on ratios of NGS paired reads) as shown in FIG. 2D. Other clones, for example clone 17, showed in-frame deletion for one allele and out-of-frame deletion for other two alleles.

3. Characterization of Ush2A knockout cell lines

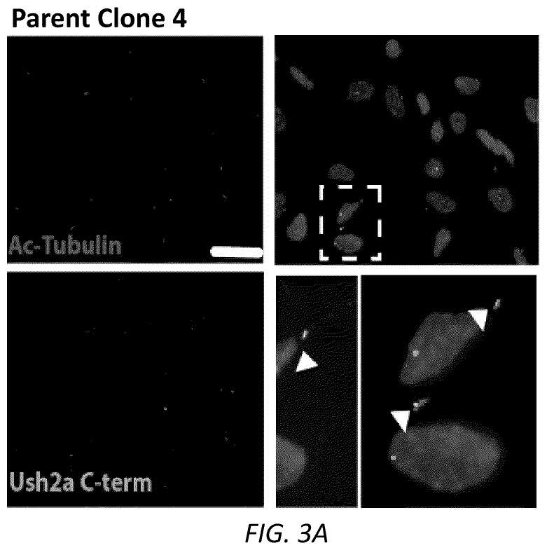

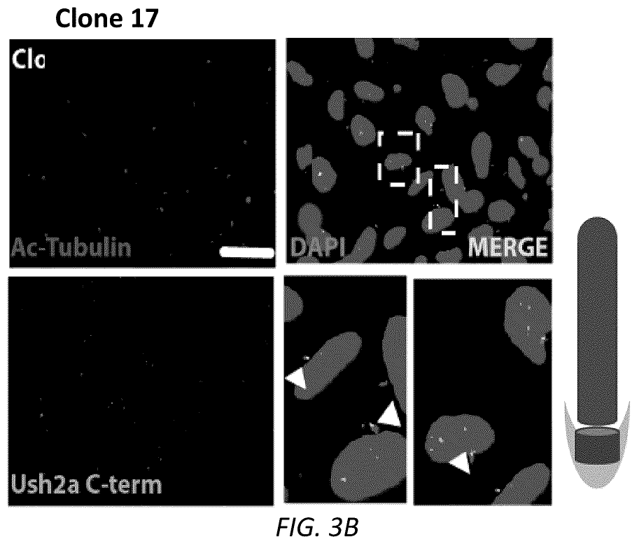

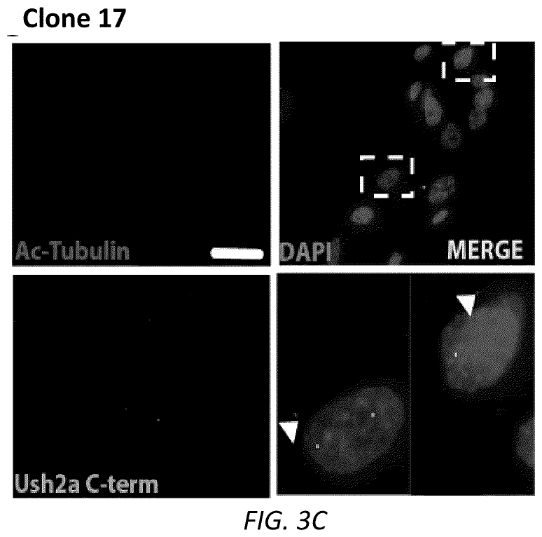

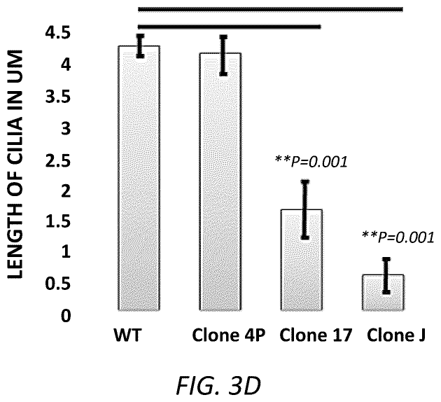

Ush2a is expressed at the base of cilia. This experiment investigated whether depletion of Ush2a would affect ciliogenesis. Cells were serum starved and stained for Ush2a and acetylated alpha-tubulin, a ciliary marker. As illustrated in FIG. 3, clone 4 and clone 17 produced cilia as shown by Ace-tubulin staining (FIGS. 3A and 3B). They also expressed Ush2a protein at base of cilium, detected by anti-C-term Ush2a antibody (Zou et al., Hum Mol Genet. 2015 Dec. 15; 24(24):6944-57). The null clone J failed to produce any detectable cilia (FIG. 3C). However, a stunted basal body look-like structure was observed in clone J. Cilia length was further measured from the basal body to the tip of cilia using an acetylated tubulin antibody. (FIG. 3D, n=50). Significant shortening of cilia in clone 17 and J, compared to the wild-type cells was observed, indicating that ciliogenesis is hampered in the Ush2a null OC-k1 cells.

4. Expression of USH2A-ΔEx13 cDNA rescued the ciliogenesis in Ush2a null cells

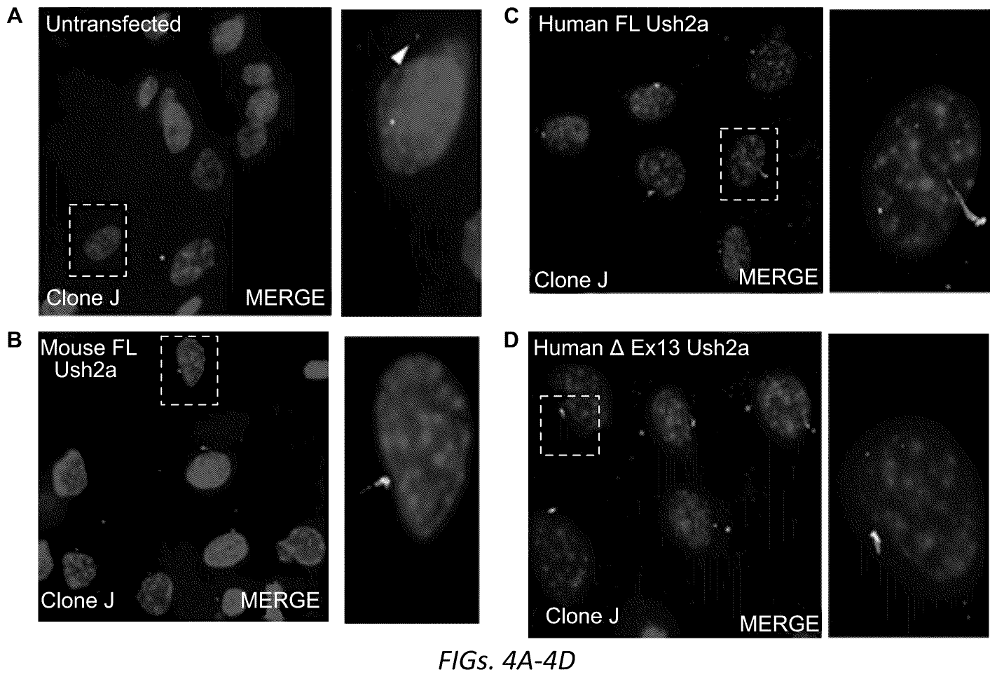

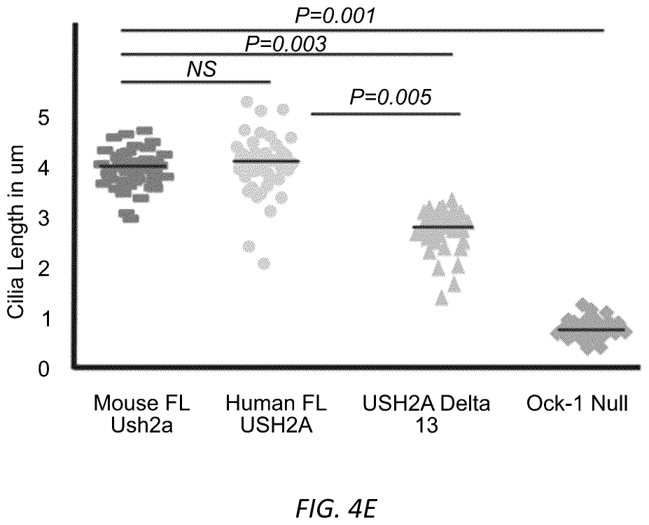

In order to determine whether the Ush2a protein that lacks portion of the reparative Laminin EGF like domain (encoded by exon 13) will retain partial or complete biologic function of USH2A protein, full-length and USH2A-ΔEx13 cDNAs were transfected into the Ush2a null line and their effect on ciliogenesis was evaluated. It was observed that both the mouse and human wild-type full length USH2A cDNAs were able to rescue ciliogenesis (FIGS. 4B and 4C). In addition, the expression of human USH2A-ΔEx13 was able to increase the length of cilia to 63% of the wild-type cilia (FIGS. 4D and 4E). These results indicate that the product of the aberrant USH2A-ΔEx13 retains at least partial biological function of USH2A.

### Example 2. Targeted Ush2a-ΔEx12 Mouse Model

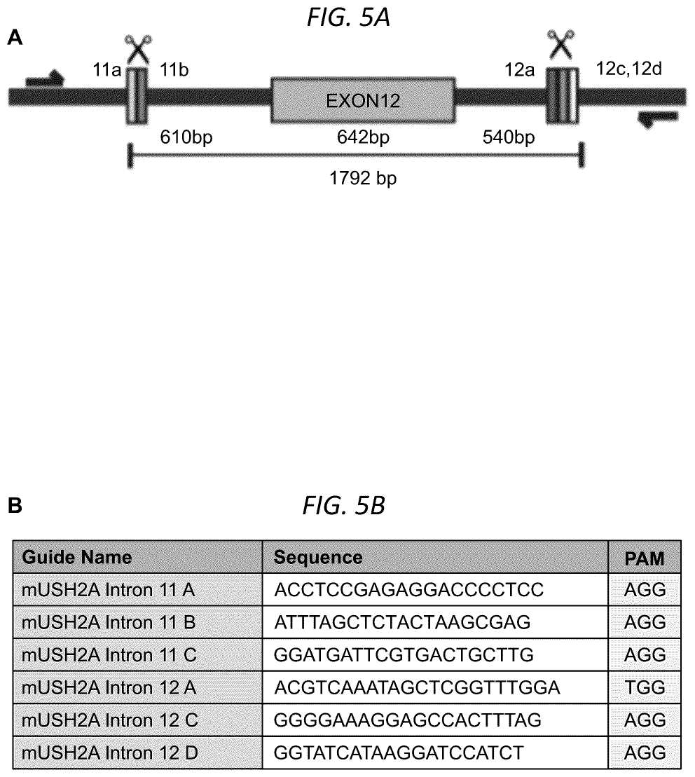

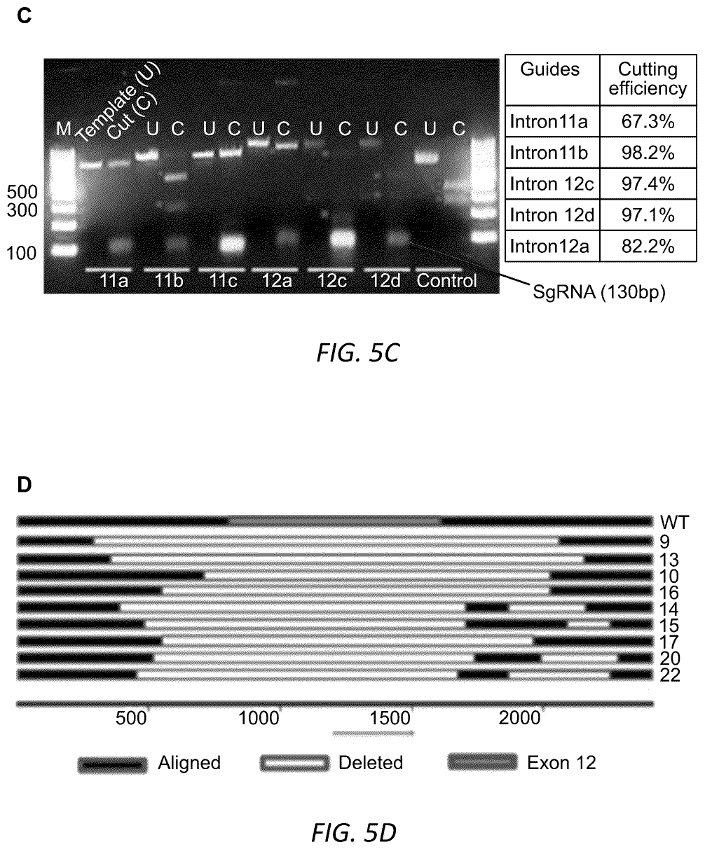

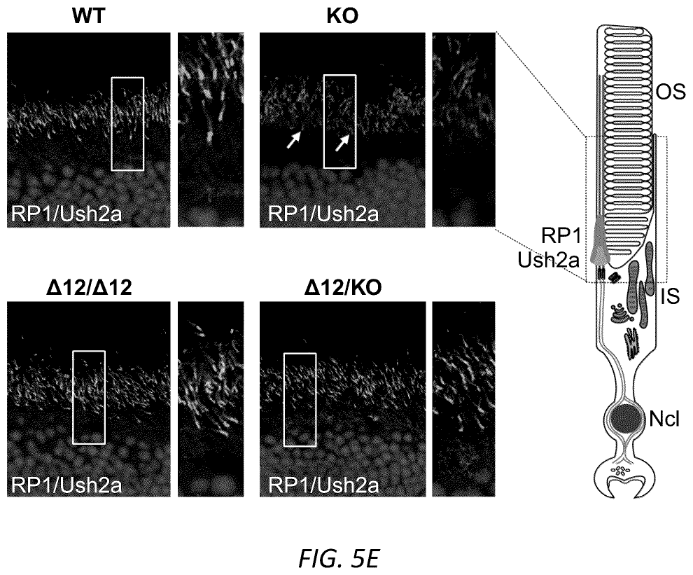

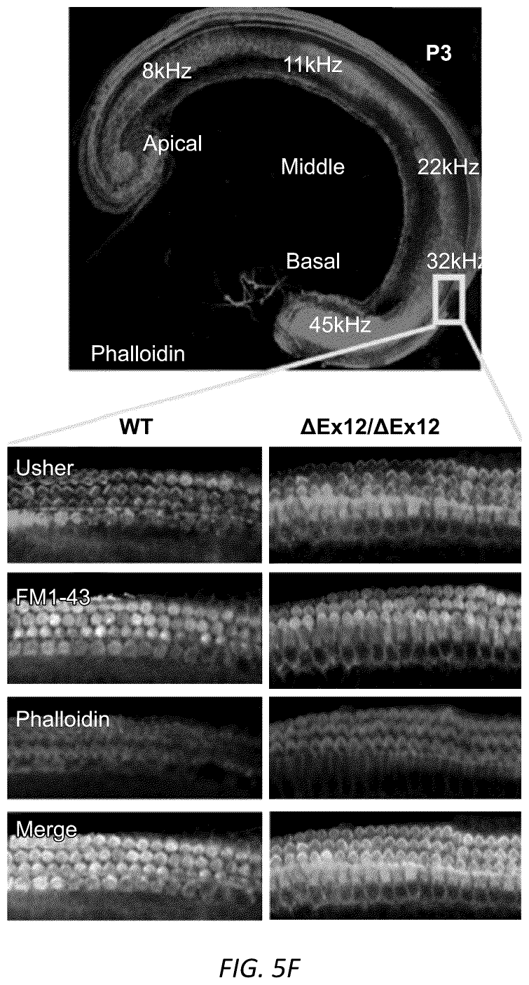

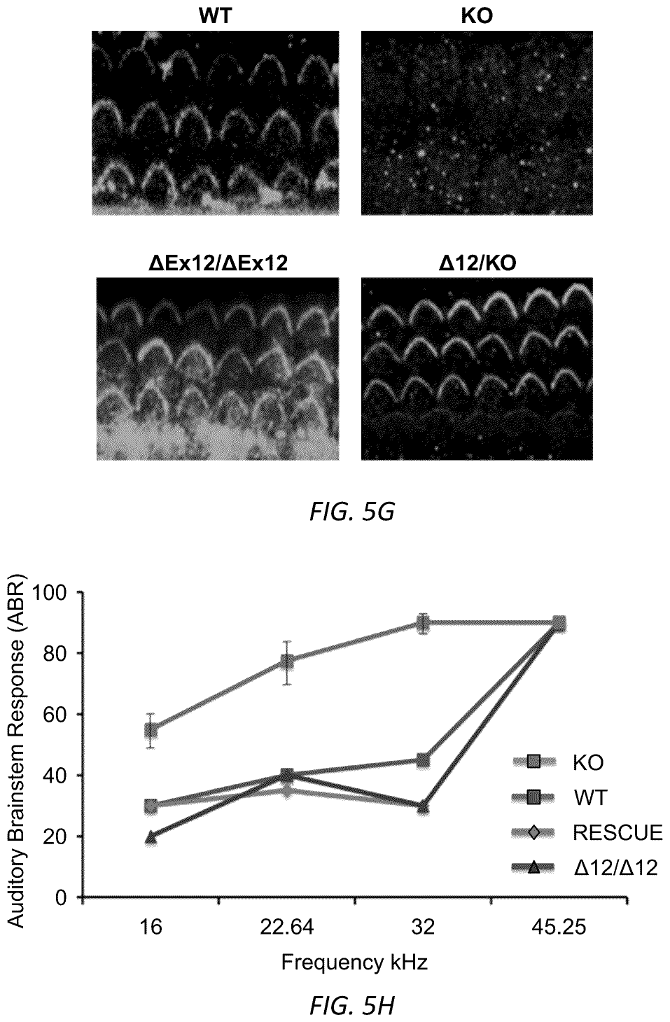

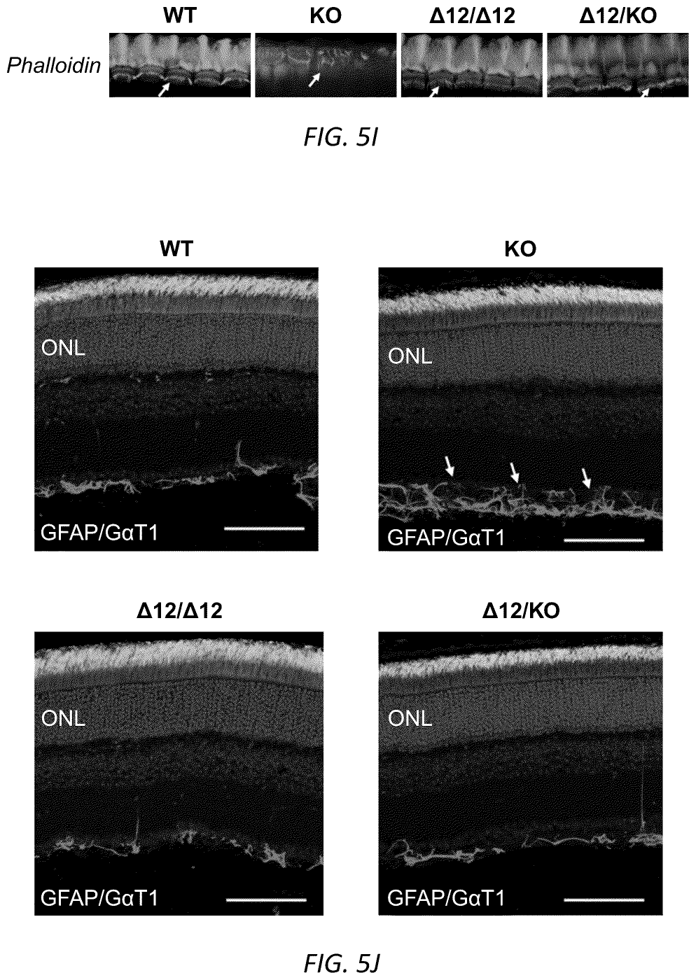

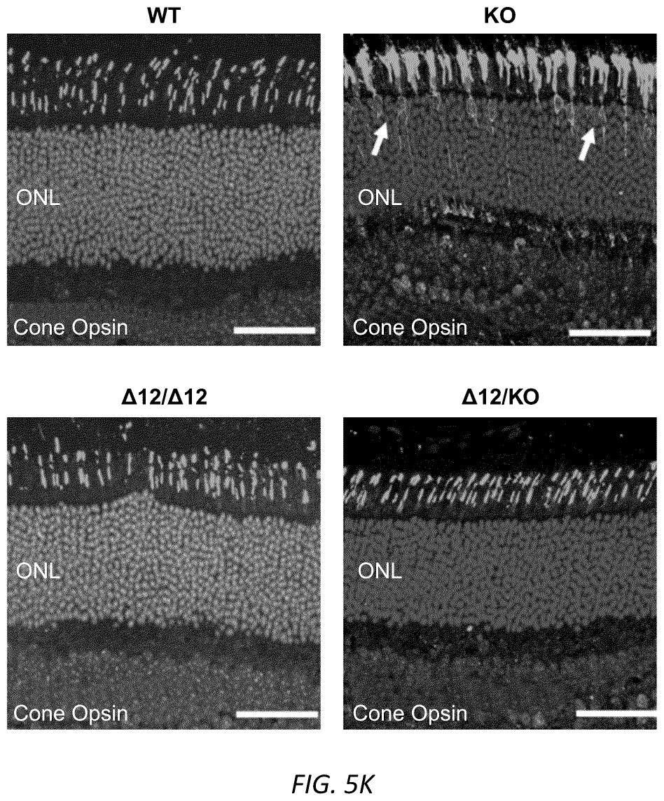

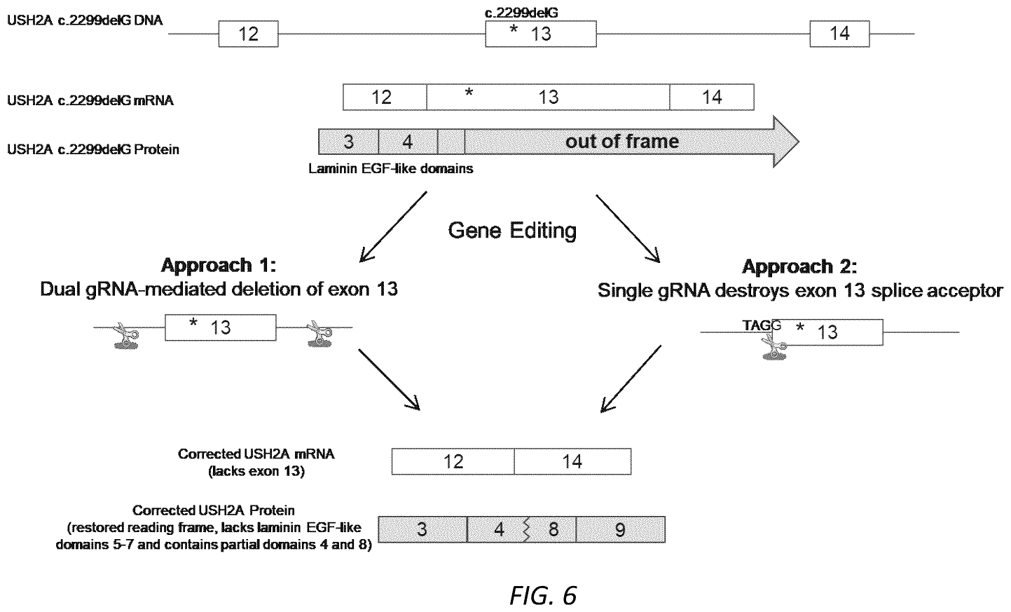

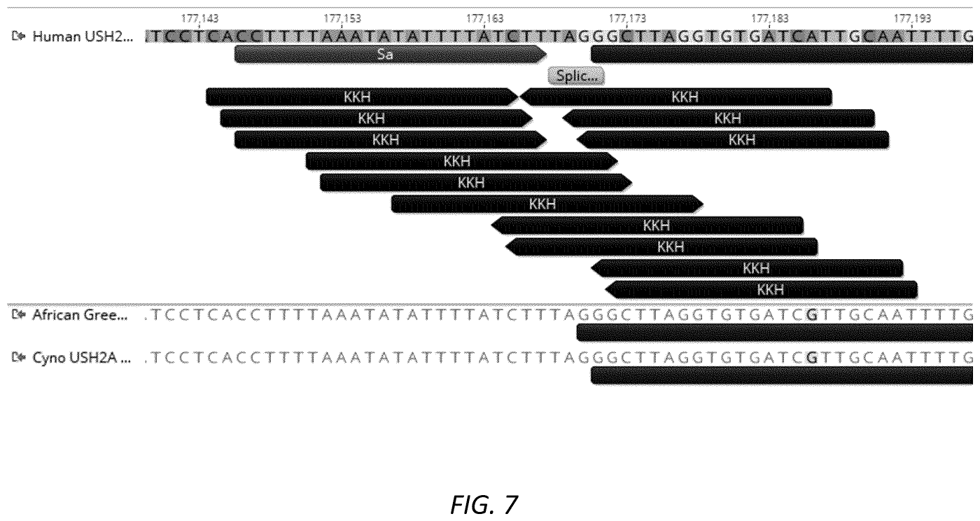

To further assess the cell-based findings in Ush2a null cells in vivo, Ush2a-ΔEx12 mouse lines were generated using CRISPR/Cas9 technology. Pairs of sgRNAs that target the flanking intron 11(11A, 11B and 11C) and intron 12 (12A, 12C and 12D) (FIG. 5A, 5B) were designed. All guides were synthesized and in vitro transcribed and tested for cleavage efficiency using an in-vitro cleavage assay. FIG. 5C illustrates the selected sgRNAs are active and cleaved PCR template derived from Ush2a genomic region surrounding exon 12 with efficiencies of 67.3% to 98.2%. All three pairs of sgRNAs were microinjected together with SpCas9 protein into the pronuclei of mouse zygotes to generate the Ush2a-ΔEx12 mouse lines in the Genome Modification Facility at Harvard University. A total of 40 pups were obtained from the pronuclear injections. With initial genotyping and sequencing, it was confirmed that 29 mice (71%) carry deletion of exon 12 and flanking introns with different sizes. Sanger sequence verified 9 founders of mice (FIG. 5D). The male founders were further backcrossed with C57BL6/J females and F1 generations were obtained.

The phenotype of the resulting Ush2a-ΔEx12 mouse lines was characterized histologically and functionally. The localization of the Ush2a-ΔEx12 protein and its interaction with other Usher2 complex proteins at two months of age was determined by immunostaining, and showed that both the wild type and exon 12-skippped Ush2a proteins were localized at the transition zone of photoreceptor sensory cilia in ΔEx12/ΔEx12, ΔEx12/ko, and wt mice (FIG. 5E).

The therapeutic potential of Ush2a-ΔEx12 was evaluated by transferring this Ush2a-ΔEx12 allele onto an Ush2a null background in Ush2a^{-/-} knockout mice (Liu, X., et al., Proc Natl Acad Sci USA, 2007. 104(11): p. 4413-8.) The phenotypes observed in these Ush2a^{-/-} mice include progressive disruption of inner hair cells in the cochlea after 4 months of age and diminished inner hair cells at 7 months of age; a detectable accumulation of GFAP and mis-localization of cone opsin at 3 months of age; gradual outer nuclear layer thinning and photoreceptor abnormalities after 10 months of age; 50% loss of photoreceptors and 60% or greater reduction of ERG amplitudes for a- and b-waves by the age of 20 months (Liu, X., et al., Proc Natl Acad Sci USA, 2007. 104(11): p. 4413-8; Lu, B., et al., Invest Ophthalmol Vis Sci, 2010. 51(4): p. 2269-76.

Cochleas were isolated from P3 ΔEx12 ΔEx12 and wt mice and stained for phalloidin (top panel), and Ush2a, FM1-43, and phalloidin (FIG. 5F), showing essentially normal morphology in the ΔEx12 ΔEx12 mice. Inner and outer hair cell structures were grossly normal in ΔEx12 ΔEx12, ΔEx12 ko, and wt mice on staining with Ush2a, as compared with the knockout (FIG. 5G). Auditory brain stem recordings showed improved hearing as determined by ABR in the Ush2a ko/ko mice by the Ush2a-Exl2 allele (FIG. 5H). Phalloidin staining showed disrupted bundles in the ko/ko mice, but not in ΔEx12 ΔEx12, ΔEx12 ko, or wt mice (FIG. 5I).

In the retina, abnormal accumulation of GFAP in Ush2a ko/ko mice was reduced in the ΔEx12 ko mice at 3 months of age, and cone opsin localization was normalized in ΔEx12 ko mice at 3 months of age, as compared to ko/ko mice (FIGS. 5J-K).

These results showed that the protein encoded by Ush2a-ΔEx12 allele rescued the cochlear and retinal phenotypes observed in Ush2a^{-/-} knockout mice.

### Example 3. Humanized USH2A Mouse Models

A humanized USH2A mouse models is developed. Exon 12 of the mouse Ush2a gene, along with up to 1500 bp of the flanking introns, is replaced with the syntenic human exon 13 and up to 1500 bp of flanking introns. Two models are generated—one in which the wildtype human exon 13 is used and one in which the human exon 13 contains the c.2299delG mutation.

The expectation is that the mouse containing the wildtype human exon 13 will be phenotypically normal. This is supported by the high level of similarity between the amino acids encoded by mouse exon 12 and human exon 13 (74.8% exact sequence identity match and 86.9% sequence similarity match based on amino acid properties).

The expectation is that the mouse containing the c.2299delG mutation will exhibit an Usher Syndrome disease phenotype. As in Usher Syndrome patients, the c.2299delG mutation will result in a frameshift and premature stop codon, leading to a prematurely truncated, and non-functional protein. This is therefore expected to mimic the phenotype of the Ush2a knockout mouse (Ush2a^{-/-}) as described above

The humanized Ush2a mouse models enable pharmacology PK/PD studies with human USH2A-targeted therapeutic guides. In addition, they enable demonstration of correction of disease phenotype. Lead gRNAs, along with Cas9 will be packaged in AAV. An example of what the configuration of this vector could look like is given in FIG. 9. Several AAV serotypes could be used, for example, AAV5, AAV8, AAV9 and AAV-Anc80 have all been demonstrated to show strong tropism for photoreceptors. lul of AAV will be delivered to mice subretinally at doses ranging from 1E+11 to 1E+13 viral genomes/mL. Mice may be treated at different ages to assess ability to reverse pathology at various stages in disease. Mice will be evaluated at several time points to assess functional and structural rescue of retinal and cochlear phenotypes. Molecular analysis will determine expression levels of Cas9 and gRNAs as well as quantify targeted gene editing rates. ERG will measure photoreceptor function in the retina. Optical coherence tomography (OCT) and histology will examine retinal structure.

### Example 4. sgRNAs for exon13 skipping in humanized USH2A mice

A comprehensive list of sgRNAs for SaCas9, SpCas9, their variants, and Cpf1 were generated to target the flanking intron 12 and 13 in the humanized USH2A mice. Those guides are individually screened in the human cell lines. Optimal pairs of sgRNAs are further evaluated for skipping the exon 13 in the humanized mice.

TABLE 4
Guide RNAs for flanking Intronic sequences of humanized exon 13

| | | Designed | | selected | |
|---|---|---|---|---|---|
| | | sgRNAs | | for testing | |
| | | Intron | Intron | Intron | Intron |
| Cas9 | PAM | 12 | 13 | 12 | 13 |
| SpCas9 | 5′-NGG-3′ | 81 | 133 | 27 | 21 |
| SpCas9-VQR | 5′-NGA-3′ | 186 | 248 | 21 | 23 |
| SpCas9-VRER | 5′-NGCG-3′ | 3 | 1 | 0 | 0 |
| SaCas9 | 5′-NNGRRT-′3 | 52 | 54 | 12 | 24 |
| SaCas9-KKH | 5′-NNNRRT-′3 | 298 | 296 | 19 | 23 |
| AsCpf1 or LbCpf1 | 5′-TTTN-3′ | 208 | 182 | 22 | 11 |
| | | 828 | 914 | 101 | 101 |

### Example 5: Screening of gRNA

Guide RNAs were screened within human USH2A intron 12 and intron 13 to find the best cutting gRNAs. To this end, ability of 41 gRNAs within intron 12 and 72 gRNAs within intron 13 to generate indels in HEK293 cells was evaluated. The cells were transfected with RNPs, gDNA was isolated 48 hours later and subjected to PCR amplification and high-throughput sequencing and analysis to determine the editing rates for each gRNA (Table 5).

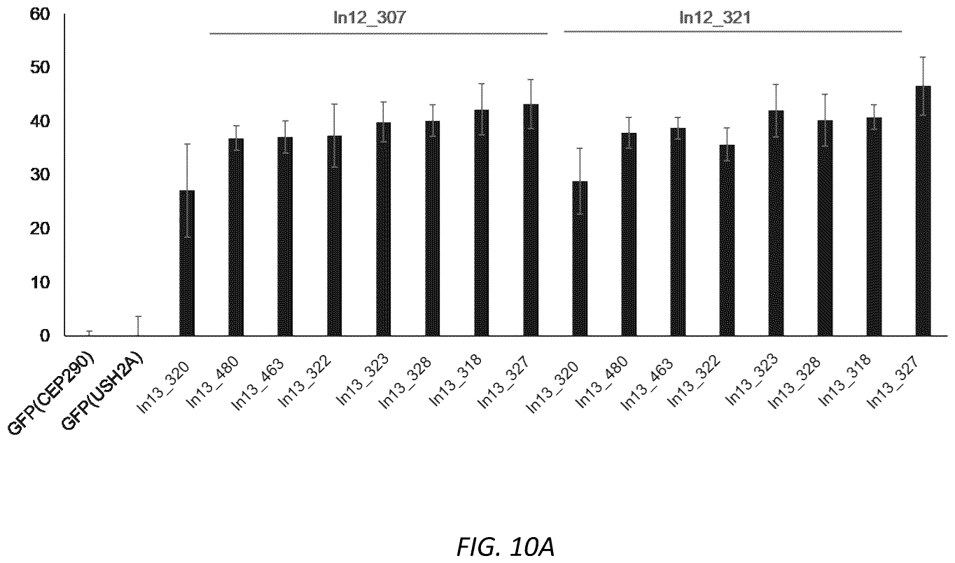

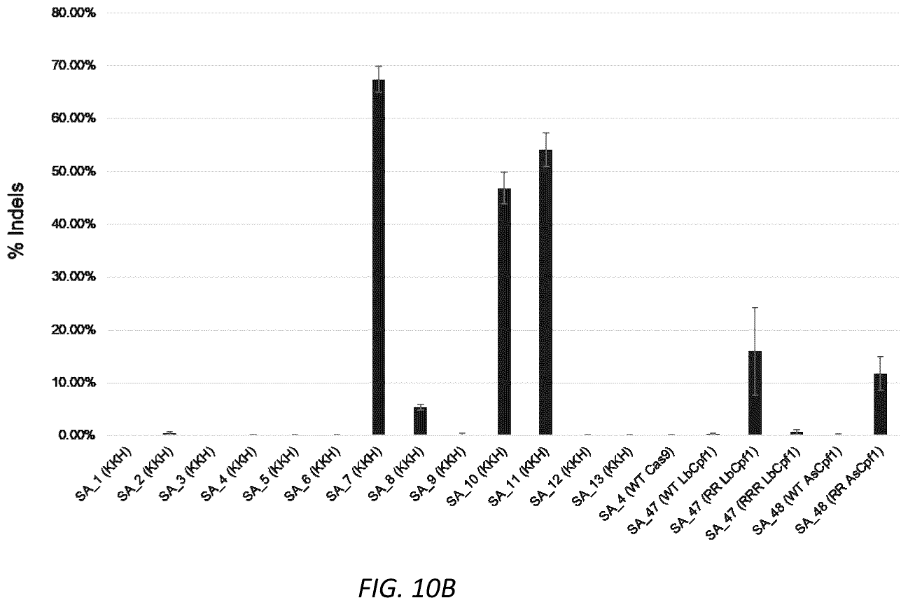

Following this initial screen, a second screen focused on 2 intron 12 gRNAs and 8 intron 13 gRNAs which were identified from the initial screen. All the possible gRNA combinations were screened to determine which worked together to give the highest loss of exon 13. U2OS cells were transfected with plasmids that expressed S. aureus Cas9 and the gRNAs of interest and gDNA was isolated 48 hours later for analysis. Editing was determined with a ddPCR assay that measures the presence or absence of USH2A exon 13. Results are shown in FIG. 10A (N=3) for all gRNA combinations as well as negative controls (GFP).

For the single gRNA approach, where the aim is to disrupt the exon 13 splice acceptor, gRNA that cut near the exon 13 splice acceptor were identified (Table 3). The ability of these gRNA to cut and form indels in U20S cells was tested through plasmid transfection of CRISPR Cas9 or Cpf1 and the associated gRNA. The genomic DNA was extracted and subject to PCR amplification and sequencing to determine the percentage of indels (FIG. 10B).

TABLE 5

| Guide | Editing |
|---|---|
| in12_67 | 0.11% |
| in12_68 | 0.68% |
| in12_69 | 0.39% |
| in12_70 | 1.68% |
| in12_71 | 0.15% |
| in12_72 | 0.34% |
| in12_73 | 0.17% |
| in12_74 | 0.16% |
| in12_75 | 0.26% |
| in12_76 | 0.15% |
| in12_77 | 0.36% |
| in12_78 | 0.09% |
| in12_79 | 0.07% |
| in12_80 | 0.11% |
| in12_81 | 0.39% |
| in12_82 | 0.68% |
| in12_83 | 0.38% |
| in12_84 | 1.66% |
| in12_85 | 0.07% |
| in12_86 | 0.13% |
| in12_87 | 1.78% |
| in12_88 | 0.64% |
| in12_89 | 0.35% |
| in12_90 | 0.57% |
| in12_307 | 17.10% |
| in12_308 | 0.30% |
| in12_309 | 11.50% |
| in12_310 | 0.26% |
| in12_311 | 1.65% |
| in12_312 | 1.90% |
| in12_313 | 0.07% |
| in12_314 | 0.20% |
| in12_315 | 7.31% |
| in12_316 | 1.70% |
| in12_317 | 0.24% |
| in12_318 | 4.26% |
| in12_319 | 0.39% |
| in12_320 | 1.69% |
| in12_321 | 6.33% |
| in12_322 | 0.42% |
| in12_323 | 1.62% |
| in13_73 | 72.91% |
| in13_74 | 0.14% |
| in13_75 | 0.12% |
| in13_76 | 0.07% |
| in13_77 | 0.26% |
| in13_78 | 73.31% |
| in13_79 | 0.09% |
| in13_80 | 1.06% |
| in13_81 | 0.33% |
| in13_82 | 0.38% |
| in13_83 | 0.25% |
| in13_84 | 0.67% |
| in13_85 | 0.21% |
| in13_86 | 73.04% |
| in13_87 | 0.10% |
| in13_88 | 73.09% |
| in13_89 | 0.32% |
| in13_90 | 73.13% |
| in13_91 | 73.07% |
| in13_92 | 0.17% |
| in13_93 | 0.20% |
| in13_94 | 0.16% |
| in13_95 | 0.17% |
| in13_96 | 1.38% |
| in13_97 | 0.18% |
| in13_98 | 0.09% |
| in13_99 | 0.42% |
| in13_100 | 0.78% |
| in13_101 | 2.17% |
| in13_102 | 0.12% |
| in13_103 | 0.51% |
| in13_104 | 0.11% |
| in13_105 | 1.33% |
| in13_106 | 73.74% |
| in13_107 | 0.20% |
| in13_314 | 0.16% |
| in13_315 | 1.99% |
| in13_316 | 4.89% |
| in13_317 | 0.18% |
| in13_318 | 9.29% |
| in13_319 | 1.75% |
| in13_320 | 11.78% |
| in13_321 | 1.95% |
| in13_322 | 10.90% |
| in13_323 | 8.37% |
| in13_324 | 1.74% |
| in13_325 | 5.81% |
| in13_326 | 1.84% |
| in13_327 | 14.96% |
| in13_328 | 7.75% |
| in13_329 | 7.98% |
| in13_330 | 0.11% |
| in13_379 | 0.20% |
| in13_384 | 0.54% |
| in13_392 | 0.97% |
| in13_393 | 0.30% |
| in13_404 | 0.84% |
| in13_405 | 73.28% |
| in13_408 | 73.14% |
| in13_417 | 2.82% |
| in13_421 | 0.79% |
| in13_437 | 1.24% |
| in13_438 | 0.84% |
| in13_452 | 0.53% |
| in13_454 | 0.13% |
| in13_460 | 73.84% |
| in13_463 | 16.13% |
| in13_466 | 73.58% |
| in13_469 | 4.02% |
| in13_472 | 0.23% |
| in13_478 | 0.09% |
| in13_480 | 18.54% |

### Example 6: Specificity of Top gRNA

The specificity of the top cutting gRNA for the dual gRNA approach was assessed using three different analyses. First, an in silico screen was conducted to identify all sites in the human genome where the particular guide could potentially cut, allowing for up to 3 mismatches or gaps in the protospacer sequence (Tables 6a and 6b).

Next, two different unbiased screens to identify off-target cut sites were completed. Guide-Seq was performed to assess the number and location of all editing events that occurred following treatment of cells with RNPs containing the one of the top gRNA. Guide-Seq was performed in primary human T cells after activation and expansion of the cells. The cells were nucleofected with RNPs and a short double-stranded oligo (Nat. Biotech. 2015, 33: 187-197). gDNA was isolated, sheared, and adapters for PCR amplification were added before PCR amplification. DNA sequences adjacent to the Guide-Seq oligo were aligned to the genome to identify the location where the double-strand oligo was inserted.

Finally, Digenome-Seq was used as a second unbiased method to locate off-target cut sites. In this method, purified genomic DNA is mixed with RNP in a cell-free system (Nat. Methods 2015, 12: 237-243). The DNA is then isolated, undergoes high-throughput sequencing, and is aligned to the human genome to identify locations where the DNA was cut (Tables 6a and 6b).

TABLE 6a

| | | # Digenome | # GUIDE-Seq | # In Silico |
|---|---|---|---|---|
| | gRNA | Off-Targets | Off-Targets | Off-Targets |
| Intron 12 | In12_307 | 1 | 0 | 208 |
| | In12_321 | 1 | 0 | 14 |

TABLE 6b

| | | # Digenome | # GUIDE-Seq | # In Silico |
|---|---|---|---|---|
| | gRNA | Off-Targets | Off-Targets | Off-Targets |
| Intron 13 | In13_318 | 9 | 0 | 400 |
| | In13_322 | 1 | 1 | 53 |
| | In13_323 | 1 | 0 | 42 |
| | In13_327 | 1 | 0 | 50 |
| | In13_328 | 2 | 1 | 47 |

### Example 7: Human Model of USH2A Editing and Exon 13 Skipping in Cells

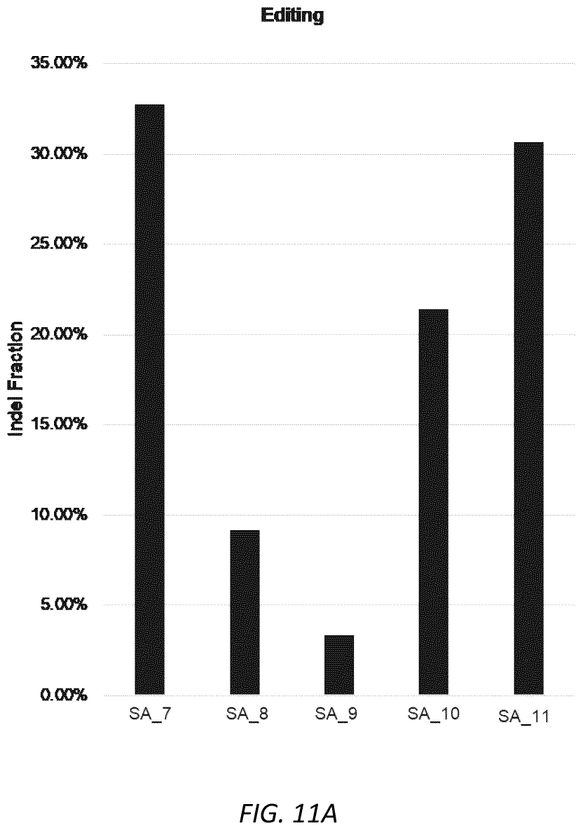

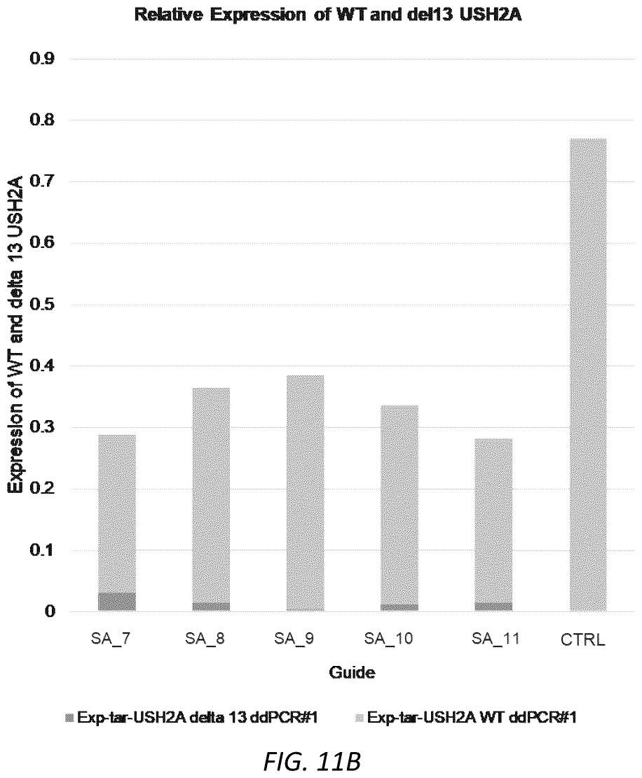

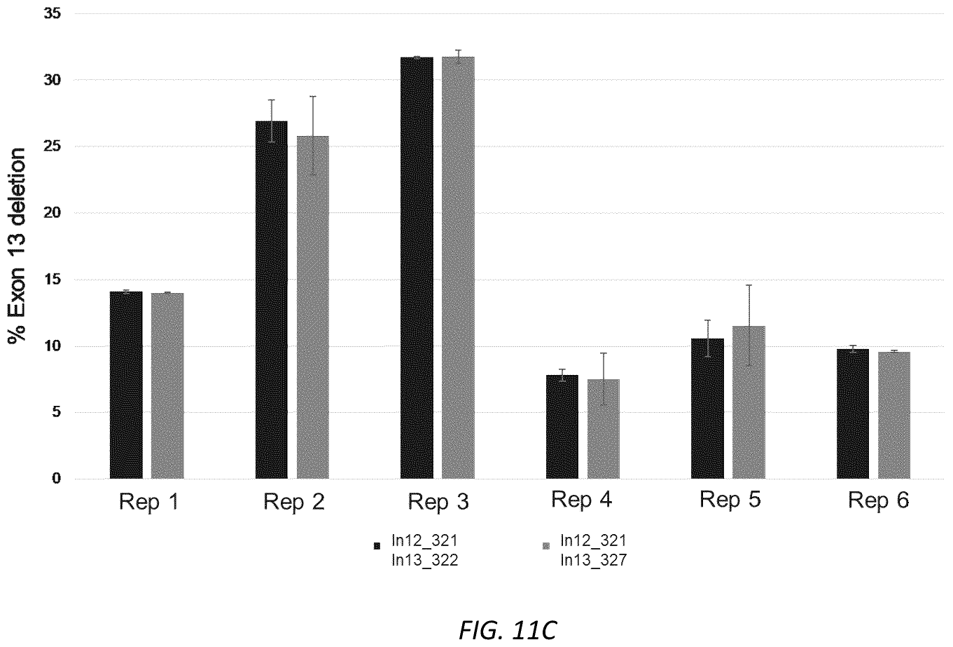

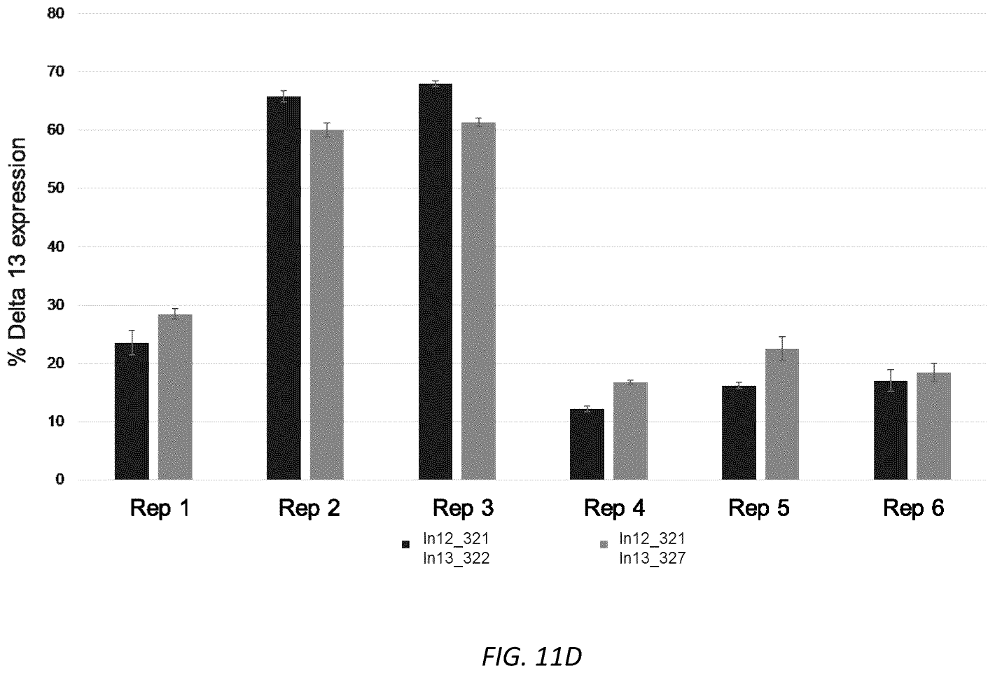

An RTddPCR assay to measure the amount of delta exon 13 USH2A transcript relative to WT USH2A was established. The WT assay amplifies the RNA junction between exons 13 and 14 while the delta 13 assay amplifies the junction between exon 12 and exon 14, which will only occur if exon 13 is precisely skipped (FIG. 11A). Another transcript ubiquitously expressed from Chromosome 1 (Clorf43) was used as a reference in this assay. The assay was validated with plasmids to check the linearity against expected inputs.

The ability of the assay to detect delta exon 13 USH2A transcripts after CRISPR/Cas9 mediated editing was tested in human CRL-5923 cells, which have been shown to express USH2A. CRL-5923 cells were transfected with plasmids expressing Cas9 and gRNA for the single guide approach and DNA and RNA were isolated from the cells 4 days after transfection. The DNA was assessed for genomic editing by high-throughput sequencing. RNA was used in the RTddPCR assay to measure the level of WT and delta exon 13 USH2A transcripts (FIGS. 11 B-C).

Editing and delta 13 USH2A expression was compared with two top pairs of gRNAs. CRL-5923 cells were nucleofected with RNPs containing the two gRNA and then DNA and RNA were isolated 4 days later (6 biological replicates). Loss of genomic USH2A exon 13 was measured by a ddPCR assay (FIG. 1ID) and levels of WT and delta exon 13 USH2A transcripts were measured by RTddPCR (FIG. 11E).

### Example 8: Editing of USH2A in human retinal explants

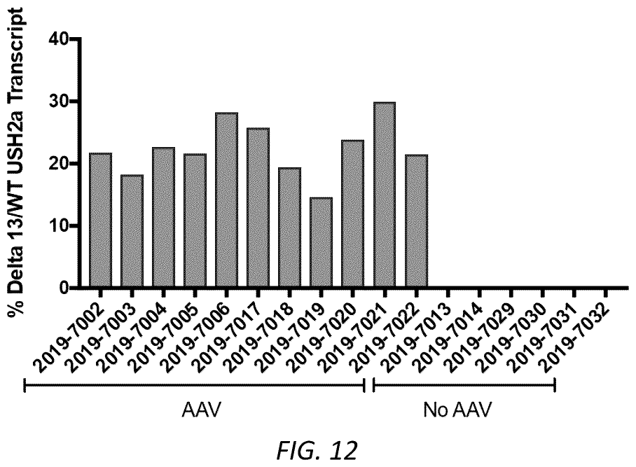

AAV5 vectors were cloned and produced to express inl2_321 and inl3_322 as depicted in FIG. 9. Retinal explant punches (3 millimeters) were taken from cadaver eyes within 5 hours post-mortem and individually cultured on membranes. 10 μL of AAV at a titer of 5e13 vg/mL was added between the neural retina and the membrane such that it created a viral bleb under the retinal tissue. Tissue was incubated for 28 days at which point the punches were collected. Each punch was split in half, one half for gDNA and the other half for RNA isolation. PCR amplification and sequencing showed that the punches treated with AAV had between 5-40% total editing, while untreated control punches had background levels of editing. RNA was used for RTddPCR analysis to determine whether editing had caused production of USH2A delta exon 13 transcripts. Analysis showed that between 14-30% of USH2A transcripts were lacking exon 13 (FIG. 12).

SEQUENCES
Human USH2A mRNA lacking exon 13: (exon 12 in bold, exon 14
double underlined)
(SEQ ID NO: 1)
TGTTTGCTCTGCAGAATACTTTACCTGGGCACCCAAGTCTTCCTTCCAGCATTCCTGCTGCTA
CAGCCTATTTGCTGAGTAACCAGGGGTTACAGCAGCGTTGCCAGGCAACGAGGGACAGCG
GTCCTGTTGAAGAGCCATTTGTCACACTGAGGGGACTGGTTGAAATGCAATAAAGAAATGA
TACCAGCAGCTACTCATGTCTTCGCCATTGCTAAGAACGTCGTTGGTATTACCTTACTCTGAG
AACGTGTCTGCAGTTTCCAGAAAATGGAGTATCGCAACATCACTTAAAGTACCCTGCTTCAA
AGTATTGCTGGCAAGTGGCGTGGGCCTGATTATTTATTTAGAAATGCTTTATCAGGAGGAG
AATGCTTTTTTGTAAACATGAATTGCCCAGTTCTTTCATTGGGCTCTGGCTTCTTGTTTCAGG
TCATTGAAATGTTGATCTTTGCCTATTTTGCTTCAATATCCTTGACTGAGTCACGAGGTCTTTT
CCCAAGGCTGGAGAACGTGGGAGCTTTCAAGAAAGTTTCCATCGTGCCAACCCAAGCAGTA
TGTGGACTCCCAGACCGAAGCACTTTTTGTCACAGCTCTGCTGCTGCTGAAAGTATTCAGTT
CTGTACCCAGCGGTTTTGTATTCAGGATTGCCCATACAGATCTTCACACCCTACCTACACTGC
CCTTTTCTCAGCAGGCCTCAGTAGCTGCATCACACCAGACAAGAATGATCTGCATCCTAACG
CCCATAGCAATTCTGCAAGTTTTATTTTTGGAAATCACAAGAGCTGCTTTTCTTCTCCTCCTTC
TCCAAAGCTGATGGCATCATTTACCTTAGCTGTATGGCTGAAACCTGAGCAACAAGGTGTAA
TGTGTGTTATAGAAAAGACAGTAGATGGGCAGATTGTGTTCAAACTTACAATATCTGAGAA
AGAGACCATGTTTTATTATCGCACAGTAAATGGTTTGCAACCTCCAATAAAAGTAATGACAC
TGGGGAGAATTCTTGTGAAGAAATGGATTCATCTTAGTGTGCAGGTGCATCAGACAAAAAT
CAGCTTCTTTATCAATGGCGTGGAGAAGGATCATACACCTTTCAATGCAAGAACTCTAAGTG
GTTCAATTACAGATTTTGCATCTGGTACTGTGCAAATAGGACAGAGTTTAAATGGTTTAGAG
CAGTTTGTCGGAAGAATGCAAGATTTTCGATTATACCAAGTGGCACTTACAAACAGAGAGA
TTCTGGAAGTCTTCTCTGGAGATCTTCTCAGATTGCATGCCCAATCACATTGCCGTTGCCCTG
GCAGCCACCCGCGGGTCCACCCTTTGGCACAGCGGTACTGCATTCCTAATGATGCAGGAGA
CACAGCTGATAATAGAGTGTCACGGTTGAATCCTGAAGCCCATCCTCTCTCTTTTGTCAATG
ATAATGATGTTGGTACTTCATGGGTTTCAAATGTGTTTACAAACATTACACAGCTTAATCAA
GGAGTGACTATTTCAGTTGATTTGGAAAATGGACAGTATCAGGTGTTTTATATTATCATTCA
GTTCTTTAGTCCACAACCAACGGAAATAAGGATTCAAAGGAAGAAGGAAAATAGTTTAGAT
TGGGAGGACTGGCAATATTTTGCCAGGAATTGTGGTGCTTTTGGAATGAAAAACAATGGAG
ATTTGGAAAAACCTGATTCTGTCAACTGTCTTCAGCTTTCCAATTTTACTCCATATTCCCGTGG
CAATGTCACATTTAGCATCCTGACACCTGGACCAAATTATCGTCCTGGATACAATAACTTCTA
TAATACCCCATCTCTTCAAGAGTTCGTAAAAGCCACGCAAATAAGGTTTCATTTTCATGGGC
AGTACTATACAACTGAGACTGCTGTTAACCTCAGACACAGATATTATGCAGTGGACGAAATC
ACCATTAGTGGGAGATGTCAGTGCCATGGTCATGCCGATAACTGCGACACAACAAGCCAGC
CATATAGATGCCTCTGCTCCCAGGAGAGCTTCACTGAAGGACTTCATTGTGATCGCTGCTTG
CCTCTTTATAATGACAAGCCTTTCCGCCAAGGTGATCAAGTTTACGCTTTCAATTGTAAACCT
TGTCAATGCAACAGCCATTCCAAAAGCTGCCATTACAACATCTCTGTAGACCCATTTCCTTTT
GAGCACTTCAGAGGGGGAGGAGGAGTTTGTGATGATTGTGAGCATAACACTACAGGAAGG
AACTGTGAGCTGTGCAAGGATTACTTTTTCCGACAAGTTGGTGCAGATCCTTCGGCCATAGA
TGTTTGCAAACCCTGTGACTGTGATACAGTTGGCACTAGAAATGGTAGCATTCTTTGTGATC
AGATTGGAGGACAGTGTAATTGTAAGAGACACGTGTCTGGCAGGCAGTGCAATCAGTGC
CAGAATGGATTCTACAATCTACAAGAGTTGGATCCTGATGGCTGCAGTCCCTGTAACTGCA
ATACCTCTGGGACAGTGGATGGAGATATTACCTGTCACCAAAATTCAGGCCAGTGCAAGT
GCAAAGCAAACGTTATTGGTTTTTATATTTCTCCAGGCAATGCCACTGGCTGCCTGCCATGC
TCATGCCATACAACTGGTGCAGTTAATCACATCTGTAATAGCCTGACTGGTCAGTGTGTTTG
CCAAGATGCTTCCATTGCTGGGCAACGTTGTGACCAATGCAAAGACCATTACTTTGGATTTG
ATCCTCAGACTGGAAGATGTCAGCCTTGTAATTGTCATCTCTCAGGAGCCTTGAATGAAACC
TGTCACTTGGTCACAGGCCAGTGTTTCTGTAAACAATTTGTCACTGGCTCAAAGTGTGATGC
TTGTGTTCCCAGTGCAAGCCACTTGGATGTCAACAATCTATTGGGTTGCAGCAAAACTCCAT
TCCAGCAACCTCCGCCCAGAGGACAAGTTCAAAGTTCTTCTGCTATCAATCTCTCCTGGAGT
CCACCTGATTCTCCAAATGCCCACTGGCTTACTTACAGTTTACTCAGGGATGGTTTTGAAATC
TACACAACAGAGGATCAATACCCATACAGTATTCAATACTTCTTAGACACAGACCTGTTACC
ATATACCAAATATTCCTATTACATTGAGACCACCAATGTGCATGGTTCAACAAGGAGTGTAG
CTGTCACTTACAAGACAAAACCAGGGGTCCCAGAGGGAAACTTGACTTTAAGTTATATCATT
CCTATTGGCTCAGACTCTGTGACACTTACCTGGACAACACTCTCAAATCAATCTGGTCCCATA
GAGAAATATATTTTGTCCTGTGCCCCTTTGGCTGGTGGTCAGCCATGTGTTTCCTACGAAGG
TCATGAAACCTCAGCTACCATCTGGAATCTGGTTCCATTTGCCAAGTACGATTTTTCTGTACA
GGCGTGTACTAGCGGGGGCTGTTTACACAGCTTGCCCATTACAGTGACCACAGCCCAGGCC
CCTCCCCAAAGACTAAGTCCACCTAAGATGCAGAAAATCAGTTCTACAGAACTTCATGTAGA
ATGGTCTCCACCAGCGGAACTAAATGGAATAATTATAAGATATGAACTATACATGAGAAGA
CTGAGATCTACTAAAGAAACCACATCTGAGGAAAGTCGAGTTTTTCAGAGCAGTGGTTGGC
TCAGTCCTCATTCATTTGTAGAATCGGCCAATGAAAATGCATTAAAACCTCCTCAAACAATG
ACAACCATCACTGGCTTGGAGCCATACACCAAGTATGAGTTCAGAGTCTTAGCTGTGAATAT
GGCTGGAAGTGTGTCTTCTGCCTGGGTCTCAGAAAGAACGGGAGAATCAGCACCTGTATTC
ATGATCCCTCCTTCAGTCTTTCCCCTCTCTTCGTACTCTCTCAATATCTCCTGGGAGAAGCCAG
CAGATAATGTTACAAGAGGAAAAGTTGTGGGGTATGACATCAATATGCTTTCTGAACAATC
ACCTCAACAGTCTATTCCCATGGCGTTTTCACAGCTGTTGCACACTGCTAAATCCCAAGAACT
ATCTTACACTGTAGAAGGACTGAAACCTTATAGGATATATGAGTTTACTATTACTCTCTGCAA
TTCAGTTGGTTGTGTGACCAGTGCTTCGGGAGCAGGACAAACTTTAGCAGCAGCACCAGCA
CAACTGAGGCCACCTCTGGTTAAAGGAATCAACAGCACAACAATCCATCTTAAGTGGTTTCC
ACCTGAAGAACTGAATGGACCCTCTCCTATATATCAGCTGGAAAGGAGAGAGTCATCTCTAC
CAGCTCTGATGACCACGATGATGAAAGGAATCCGTTTCATAGGAAATGGGTATTGTAAATTT
CCCAGCTCCACTCACCCAGTCAATACAGACTTCACTGGCATTAAGGCCAGCTTTCGAACAAA
AGTGCCTGAAGGTTTGATTGTCTTTGCAGCATCACCTGGCAATCAGGAAGAGTATTTTGCAC
TTCAGTTGAAGAAGGGACGTCTTTATTTTCTTTTTGATCCTCAGGGGTCACCAGTGGAAGTA
ACTACAACTAATGATCATGGCAAACAATATAGTGATGGAAAATGGCATGAAATAATTGCTA
TTAGGCATCAGGCTTTTGGCCAAATCACTCTGGATGGGATATATACAGGTTCCTCTGCCATC
CTGAATGGTAGTACTGTTATTGGAGATAACACAGGAGTCTTTCTGGGAGGGCTCCCGCGAA
GTTATACCATCCTCAGGAAGGATCCTGAGATAATCCAAAAAGGTTTTGTGGGCTGTCTCAAG
GATGTACATTTTATGAAGAATTACAATCCGTCAGCTATTTGGGAACCTCTGGATTGGCAGAG
TTCTGAAGAACAAATCAACGTGTATAACAGCTGGGAGGGATGTCCCGCTTCATTAAATGAG
GGAGCTCAGTTCCTAGGAGCAGGGTTCCTGGAACTTCATCCATATATGTTTCATGGTGGAAT
GAACTTTGAGATTTCCTTTAAGTTCAGAACTGACCAATTAAATGGATTGCTTCTTTTCGTTTA
TAACAAAGATGGACCTGATTTTCTTGCTATGGAGCTGAAAAGTGGAATATTGACCTTCCGGT
TAAATACCAGTCTTGCCTTTACACAAGTGGATCTATTGCTGGGGCTATCCTATTGTAATGGA
AAGTGGAATAAAGTCATTATTAAAAAGGAAGGCTCTTTCATATCAGCAAGTGTGAATGGAC
TGATGAAGCATGCATCGGAGTCCGGAGACCAGCCACTGGTGGTGAATTCACCAGTTTATGT
GGGAGGAATCCCACAGGAACTGCTGAACTCTTATCAACATTTGTGTTTGGAACAAGGTTTC
GGTGGTTGCATGAAGGATGTTAAATTTACACGGGGTGCTGTCGTTAACTTGGCATCTGTGTC
CAGCGGTGCTGTCAGAGTCAATCTGGATGGATGCCTATCAACTGACAGTGCTGTTAACTGC
AGGGGAAATGACTCCATCCTGGTTTACCAGGGAAAAGAGCAGAGTGTTTACGAGGGTGGT
CTCCAGCCTTTTACAGAATACCTGTATCGAGTGATAGCCTCGCATGAAGGAGGTTCAGTATA
TAGTGATTGGAGTCGAGGACGTACAACAGGAGCAGCTCCACAAAGTGTGCCAACTCCCTCA
AGAGTCCGCAGCTTAAATGGATACAGCATTGAGGTGACCTGGGATGAACCTGTTGTCAGAG
GTGTAATTGAGAAGTACATTCTGAAAGCCTATAGTGAGGACAGCACCCGTCCACCCCGCAT
GCCCTCTGCCAGTGCTGAATTTGTCAATACAAGCAACCTCACAGGCATATTGACAGGCTTGC
TACCCTTCAAAAACTATGCAGTAACCCTAACTGCTTGCACTTTGGCTGGCTGTACTGAGAGC
TCACATGCATTGAACATCTCTACTCCACAAGAAGCCCCACAAGAGGTTCAGCCACCAGTAGC
CAAATCCCTTCCCAGTTCTTTGCTGCTCTCCTGGAACCCACCCAAAAAGGCAAATGGTATTAT
AACTCAGTACTGTTTATACATGGATGGGAGGCTGATCTATTCAGGCAGTGAGGAGAACTAC
ACAGTCACAGATTTAGCAGTATTTACACCCCACCAGTTTCTACTAAGTGCATGCACACATGT
GGGCTGTACAAACAGTTCCTGGGTCCTACTGTACACAGCACAGCTGCCACCAGAACACGTG
GATTCCCCAGTTCTGACTGTCCTGGATTCTAGAACTATACACATACAGTGGAAACAACCAAG
AAAAATAAGTGGGATTCTGGAACGCTATGTATTATATATGTCAAACCATACACATGATTTTA
CAATTTGGAGTGTCATCTATAACAGTACAGAACTTTTCCAGGATCATATGCTACAATACGTTT
TACCTGGTAATAAATATCTCATCAAGCTGGGAGCTTGCACAGGTGGTGGGTGCACAGTGAG
TGAGGCCAGTGAGGCCCTAACTGACGAGGACATACCCGAAGGCGTGCCAGCCCCCAAAGC
CCACTCATATTCACCTGACTCCTTTAATGTCTCCTGGACTGAGCCTGAATATCCGAATGGTGT
TATCACGAGTTATGGATTATATCTAGATGGTATATTAATCCACAATTCCTCAGAACTCAGCTA
TCGTGCTTACGGATTTGCTCCTTGGAGTTTACATTCCTTCAGAGTCCAAGCATGCACGGCCA
AAGGTTGTGCTCTGGGCCCACTGGTGGAAAATCGAACTCTAGAAGCTCCTCCTGAAGGAAC
AGTAAATGTGTTTGTCAAAACACAGGGATCCCGGAAAGCCCACGTGAGGTGGGAAGCACC
TTTTCGCCCTAATGGACTCTTAACACACTCAGTCCTTTTCACTGGGATATTCTATGTAGACCC
AGTAGGTAATAACTACACCCTTCTGAATGTCACAAAAGTCATGTACAGCGGAGAAGAGACA
AACCTTTGGGTGCTCATCGATGGGCTGGTTCCTTTTACCAACTATACTGTACAAGTGAATATT
TCAAATAGCCAAGGCAGCTTGATAACTGATCCTATAACAATTGCAATGCCTCCAGGAGCTCC
AGATGGCGTGCTGCCTCCCAGGCTTTCATCTGCCACTCCAACCAGTCTTCAGGTTGTCTGGT
CTACACCAGCTCGTAATAACGCTCCTGGCTCTCCCAGATACCAACTCCAGATGAGGTCTGGC
GACTCCACCCATGGATTTCTAGAGTTATTTTCCAATCCTTCTGCATCGTTAAGCTATGAAGTG
AGTGATCTCCAACCGTACACAGAGTATATGTTTCGGTTGGTTGCCTCCAATGGATTTGGCAG
TGCACATAGTTCTTGGATTCCATTCATGACCGCAGAGGACAAACCTGGACCTGTAGTTCCTC
CGATTCTTCTGGATGTGAAGTCAAGAATGATGTTGGTCACCTGGCAGCATCCTAGAAAATCC
AATGGGGTTATTACCCATTATAACATTTATCTACATGGCCGTCTATACTTGAGAACTCCTGGA
AATGTCACTAATTGCACAGTGATGCATTTACACCCATACACTGCCTATAAGTTTCAGGTAGA
AGCCTGCACTTCAAAAGGATGTTCCCTTTCACCAGAGTCCCAGACTGTATGGACACTCCCAG
GGGCACCGGAAGGGATCCCAAGTCCAGAGCTGTTCTCTGATACTCCAACATCTGTGATTATA
TCTTGGCAACCCCCTACCCACCCCAATGGCTTGGTGGAGAATTTCACAATTGAGAGAAGAGT
CAAAGGAAAGGAAGAAGTTACTACCCTGGTGACTCTCCCGAGGAGTCATTCCATGAGGTTT
ATTGACAAGACTTCTGCTCTTAGCCCATGGACAAAATATGAATATCGGGTACTGATGAGCAC
TCTTCATGGAGGCACAAACAGCAGTGCTTGGGTAGAAGTTACCACAAGACCCTCACGACCT
GCTGGGGTGCAGCCACCTGTGGTGACAGTGCTGGAACCCGATGCAGTCCAGGTCACTTGG
AAACCCCCACTCATCCAGAACGGAGACATACTTAGCTATGAGATTCACATGCCTGACCCTCA
CATCACTTTAACCAATGTGACTTCCGCAGTGTTAAGTCAAAAAGTTACTCATCTGATTCCTTT
CACTAATTATTCTGTCACCATTGTTGCTTGCTCAGGGGGTAATGGGTACCTTGGAGGGTGCA
CAGAGAGTTTACCTACCTATGTTACCACTCACCCCACCGTACCTCAGAATGTTGGCCCATTGT
CTGTGATTCCACTAAGTGAATCATATGTTGTGATTTCTTGGCAACCACCATCCAAGCCAAAT
GGACCTAATTTGAGATATGAGCTTCTGAGACGTAAAATCCAGCAGCCACTTGCATCAAATCC
CCCAGAAGATTTAAATCGGTGGCACAATATTTATTCAGGAACTCAGTGGCTTTATGAAGATA
AGGGTCTTAGCAGGTTTACAACCTATGAATATATGCTCTTCGTACACAACAGTGTGGGTTTT
ACACCGAGCCGAGAAGTGACTGTGACAACGTTAGCTGGTCTTCCAGAGAGAGGAGCCAAT
CTCACTGCGAGTGTCCTTAACCACACAGCCATCGACGTGAGGTGGGCTAAACCAACTGTTCA
AGACCTACAAGGTGAAGTTGAATATTACACACTTTTTTGGAGTTCTGCTACCTCAAACGACT
CTCTAAAAATCTTGCCAGATGTAAACTCTCATGTCATTGGCCACCTAAAGCCAAACACAGAG
TATTGGATCTTTATCTCTGTCTTCAATGGAGTCCACAGCATCAACAGTGCAGGACTTCATGCA
ACCACTTGCGATGGGGAGCCTCAGGGCATGCTTCCTCCAGAGGTTGTCATCATCAACAGTA
CAGCTGTACGTGTCATCTGGACATCTCCTTCAAACCCAAATGGTGTTGTCACTGAGTATTCTA
TCTATGTAAATAATAAGCTCTACAAGACTGGAATGAATGTGCCTGGGTCGTTTATTCTGAGA
GACCTGTCTCCCTTCACTATCTATGACATTCAGGTTGAAGTCTGCACAATATATGCCTGCGTG
AAAAGCAATGGAACCCAAATTACCACTGTGGAAGACACTCCAAGTGATATACCAACACCCA
CAATTCGTGGCATCACTTCAAGATCTCTTCAAATTGATTGGGTGTCTCCACGGAAGCCAAAT
GGCATCATTCTTGGATATGATCTCCTATGGAAAACATGGTATCCATGCGCTAAAACTCAAAA
GTTAGTGCAGGATCAGAGTGATGAGCTCTGCAAGGCAGTGAGGTGTCAAAAACCTGAATCT
ATCTGTGGACACATTTGCTATTCTTCTGAAGCTAAGGTTTGTTGTAACGGAGTGCTCTATAAC
CCCAAGCCTGGACATCGCTGTTGTGAAGAAAAGTATATCCCGTTTGTTCTGAATTCTACTGG
AGTTTGTTGTGGTGGCCGAATACAGGAGGCACAACCAAATCATCAGTGCTGCTCTGGGTAT
TACGCTAGAATTCTACCAGGTGAAGTATGCTGTCCAGATGAACAGCACAATCGGGTTTCTGT
TGGCATTGGTGATTCCTGCTGTGGCAGAATGCCGTACTCCACCTCAGGAAACCAGATTTGCT
GTGCTGGGAGGCTTCATGATGGCCATGGCCAGAAGTGCTGTGGCAGACAGATTGTGAGCA
ACGATTTAGAGTGTTGTGGTGGAGAAGAAGGAGTGGTGTACAATCGCCTTCCAGGTATGTT
CTGTTGTGGGCAGGATTATGTGAATATGTCAGATACCATATGCTGCTCAGCTTCCAGTGGAG
AGTCTAAAGCACATATTAAAAAGAATGACCCGGTGCCAGTAAAATGCTGTGAGACTGAACT
TATTCCAAAGAGCCAGAAATGCTGTAATGGAGTTGGATATAATCCTTTGAAATATGTTTGCT
CTGACAAGATTTCAACTGGAATGATGATGAAGGAAACCAAAGAGTGCAGGATCCTCTGCCC
AGCATCTATGGAAGCCACAGAACATTGTGGCAGGTGTGACTTCAACTTTACCAGCCACATTT
GCACTGTGATAAGAGGGTCTCACAATTCCACAGGGAAGGCATCAATTGAAGAAATGTGTTC
ATCTGCCGAAGAAACCATTCATACAGGGAGTGTAAACACGTACTCTTACACAGATGTGAAC
CTCAAGCCCTACATGACATATGAGTACAGGATTTCTGCCTGGAACAGCTATGGGCGAGGAC
TCAGCAAAGCTGTGAGAGCCAGAACAAAAGAAGATGTGCCTCAAGGAGTGAGTCCCCCTA
CGTGGACCAAAATAGACAATCTTGAAGATACAATTGTCTTAAACTGGAGAAAACCTATACA
ATCAAATGGTCCTATTATTTACTACATCCTTCTTCGAAATGGAATTGAACGTTTTCGGGGAAC
ATCACTGAGCTTCTCTGATAAAGAGGGAATTCAACCATTTCAGGAATATTCATATCAGCTGA
AAGCTTGCACGGTTGCTGGCTGTGCCACCAGTAGCAAGGTAGTTGCAGCTACTACCCAAGG
AGTTCCGGAGAGCATCCTGCCACCAAGCATCACAGCCCTAAGTGCAGTGGCTCTGCATCTG
AGCTGGAGTGTCCCTGAGAAATCAAACGGCGTCATTAAAGAGTACCAGATCAGGCAGGTTG
GGAAAGGTCTCATCCACACTGACACCACTGACAGGAGACAGCATACGGTCACAGGTCTCCA
GCCATACACCAACTACAGCTTCACTCTTACAGCTTGTACATCTGCTGGGTGCACTTCAAGCG
AGCCTTTTCTAGGTCAGACACTGCAGGCAGCTCCTGAAGGAGTTTGGGTGACACCTCGACA
CATTATCATCAATTCTACAACAGTGGAATTATATTGGAGTCTGCCAGAAAAGCCCAATGGCC
TCGTTTCTCAATATCAATTGAGTCGTAATGGAAACTTGCTTTTCCTGGGTGGCAGTGAGGAG
CAGAATTTCACTGATAAAAACCTGGAGCCCAATAGCAGATACACTTACAAGTTAGAAGTCA
AAACTGGAGGTGGCAGCAGTGCTAGTGATGATTACATTGTTCAAACACCTATGTCAACACC
AGAAGAAATCTATCCTCCATATAATATCACAGTAATTGGGCCTTATTCTATATTTGTAGCTTG
GATACCACCAGGGATCCTCATCCCCGAAATTCCTGTGGAGTACAATGTCTTACTCAATGATG
GAAGTGTAACACCTCTGGCCTTCTCCGTTGGTCATCATCAATCCACCCTTCTGGAAAATTTGA
CTCCATTCACACAGTATGAGATAAGGATACAAGCATGTCAAAATGGAAGTTGTGGAGTTAG
CAGTAGGATGTTTGTCAAAACACCTGAAGCAGCCCCAATGGATCTTAATTCTCCTGTTCTTA
AGGCACTGGGGTCAGCTTGCATAGAGATTAAGTGGATGCCACCTGAAAAACCAAATGGAAT
CATCATCAACTACTTTATTTACAGACGCCCTGCTGGCATTGAAGAGGAGTCTGTTTTATTTGT
CTGGTCAGAAGGAGCCCTTGAATTTATGGATGAAGGAGACACCCTGAGGCCTTTCACACTC
TACGAATATCGGGTCAGAGCCTGTAACTCCAAGGGTTCAGTGGAGAGTCTGTGGTCATTAA
CACAAACTCTGGAAGCTCCACCTCAAGATTTTCCAGCTCCTTGGGCTCAAGCCACGAGTGCT
CATTCAGTTCTGTTGAATTGGACAAAGCCAGAATCTCCCAATGGCATTATCTCCCATTACCGT
GTGGTCTACCAGGAGAGACCCGACGATCCTACATTTAACAGCCCTACCGTGCATGCTTTCAC
AGTGAAGGGAACAAGCCATCAAGCCCACCTGTACGGGTTAGAACCATTCACAACATATCGC
ATTGGTGTTGTGGCTGCAAACCATGCAGGAGAAATTTTAAGCCCTTGGACTCTGATTCAAAC
CTTAGAATCTTCCCCAAGTGGACTGAGAAACTTTATAGTAGAACAGAAAGAGAATGGCCGG
GCATTGCTACTACAGTGGTCAGAACCTATGAGAACCAATGGTGTGATTAAGACATACAACA
TCTTCAGTGACGGGTTCCTGGAGTACTCTGGTTTGAATCGTCAGTTTCTCTTCCGCCGCCTGG
ATCCTTTCACTCTCTACACACTGACCCTGGAGGCCTGCACCAGAGCAGGTTGTGCACACTCG
GCGCCTCAGCCTCTGTGGACAGATGAAGCCCCTCCAGACTCTCAGCTGGCTCCTACTGTCCA
CTCTGTGAAGTCCACCAGTGTTGAGCTGAGCTGGTCTGAGCCTGTTAACCCAAATGGAAAA
ATAATTCGCTATGAAGTGATTCGCAGATGCTTCGAGGGAAAAGCTTGGGGAAATCAGACGA
TCCAGGCCGACGAGAAAATTGTTTTCACAGAATATAACACTGAAAGGAATACATTTATGTAT
AATGACACAGGTTTGCAACCATGGACGCAGTGTGAATATAAAATCTACACTTGGAATTCAG
CTGGGCATACCTGTAGCTCTTGGAATGTGGTGAGGACATTGCAAGCACCTCCAGAAGGTCT
CTCTCCACCTGTGATATCCTATGTTTCTATGAATCCCCAAAAACTGCTGATTTCCTGGATCCC
ACCAGAACAGTCTAATGGTATTATCCAGTCCTATAGGCTTCAAAGGAATGAAATGCTCTATC
CTTTTAGCTTTGATCCTGTGACTTTCAATTACACTGATGAAGAGCTTCTTCCTTTTTCCACCTA
TAGCTATGCACTCCAAGCCTGCACGAGTGGAGGATGCTCCACCAGCAAACCCACCAGCATC
ACAACTCTGGAGGCTGCTCCATCAGAAGTCAGCCCTCCAGATCTTTGGGCCGTCAGTGCCAC
TCAAATGAATGTATGTTGGTCACCGCCCACAGTGCAAAATGGAAAGATTACTAAATATTTAG
TTAGATATGATAATAAAGAGTCCCTTGCTGGCCAGGGCCTGTGCCTGCTGGTTTCCCACCTG
CAGCCTTACTCTCAGTATAACTTCTCCCTTGTAGCCTGCACGAATGGAGGTTGCACAGCTAG
TGTGTCAAAATCTGCCTGGACAATGGAGGCCCTGCCAGAGAACATGGACTCTCCAACATTG
CAAGTCACAGGCTCAGAATCAATAGAAATCACCTGGAAACCTCCAAGAAACCCAAATGGCC
AGATCAGAAGTTATGAACTTAGGAGGGATGGAACCATTGTATATACAGGCTTGGAAACACG
CTATCGTGATTTTACTCTCACCCCAGGTGTGGAGTATAGCTACACAGTAACTGCCAGCAACA
GCCAAGGGGGTATTTTGAGTCCTCTTGTCAAAGATCGAACCAGCCCCTCAGCACCCTCAGG
GATGGAACCTCCAAAATTGCAGGCCAGGGGTCCTCAGGAGATCTTAGTGAACTGGGACCCT
CCAGTGAGAACAAATGGTGATATCATCAATTATACCCTCTTCATCCGTGAACTATTTGAAAG
AGAAACTAAAATCATACACATAAACACAACTCATAATTCTTTTGGTATGCAGTCATATATAGT
AAACCAGCTGAAGCCATTTCACAGGTATGAAATACGAATTCAAGCGTGCACCACCCTGGGA
TGTGCATCAAGTGACTGGACATTCATACAGACCCCTGAGATTGCACCTTTGATGCAACCCCC
TCCACATCTGGAGGTACAAATGGCTCCAGGAGGATTCCAGCCAACTGTTTCTCTTTTGTGGA
CAGGACCGCTGCAGCCAAATGGAAAAGTTTTGTATTACGAATTATACAGAAGACAAATAGC
AACTCAGCCTAGAAAATCCAATCCAGTCCTAATCTATAACGGAAGCTCAACATCTTTTATAG
ATTCCGAACTATTGCCTTTCACAGAGTATGAGTATCAGGTCTGGGCAGTGAATTCTGCAGGA
AAAGCCCCCAGTAGCTGGACATGGTGCAGAACCGGGCCAGCCCCACCAGAAGGTCTCAGA
GCCCCCACGTTCCATGTGATCTCTTCTACCCAAGCAGTGGTCAACATCAGTGCCCCTGGGAA
GCCCAACGGGATCGTCAGTCTCTACAGGCTGTTCTCCAGCAGCGCCCATGGGGCTGAGACA
GTGCTATCCGAAGGCATGGCCACCCAGCAGACTCTCCATGGCCTTCAAGCCTTCACTAACTA
CTCTATTGGAGTAGAGGCCTGCACCTGCTTCAACTGTTGCAGCAAAGGACCGACAGCTGAA
CTGAGAACCCATCCTGCCCCACCCTCAGGACTGTCCTCTCCACAAATCGGGACGCTGGCCTC
AAGGACGGCCTCCTTCCGGTGGAGTCCCCCCATGTTCCCCAATGGTGTCATTCACAGCTATG
AACTCCAATTCCACGTGGCTTGCCCTCCTGACTCAGCCCTCCCCTGTACTCCCAGCCAAATAG
AAACAAAGTACACGGGGCTGGGGCAGAAAGCCAGCCTTGGGGGTCTCCAGCCCTACACCA
CATACAAGCTGAGAGTGGTGGCACACAACGAGGTGGGCAGTACGGCTTCCGAGTGGATCA
GTTTCACCACCCAAAAAGAATTGCCTCAGTACCGAGCCCCATTTTCGGTGGACAGCAATTTG
TCTGTGGTGTGTGTGAACTGGAGTGACACCTTCCTCCTGAACGGCCAACTGAAGGAGTACG
TGTTAACCGACGGAGGGCGACGCGTGTACAGCGGCTTGGACACCACCCTCTACATACCGAG
AACGGCGGACAAAACCTTCTTTTTCCAGGTCATCTGCACGACTGACGAAGGAAGTGTTAAG
ACGCCGTTGATCCAATATGATACCTCTACTGGACTTGGCTTGGTCCTAACAACTCCTGGGAA
AAAGAAGGGATCGCGGAGCAAAAGCACAGAGTTCTACAGCGAGCTGTGGTTCATAGTGTT
AATGGCGATGCTGGGCTTGATCTTGTTGGCCATTTTTCTGTCCCTGATACTACAAAGAAAAA
TCCACAAAGAGCCATATATCAGAGAAAGACCTCCCTTGGTACCTCTTCAGAAGAGGATGTCT
CCATTGAATGTTTACCCACCGGGGGAAAACCATATGGGGTTAGCCGATACCAAAATTCCCC
GGTCTGGGACACCTGTGAGTATCCGCAGCAACCGGAGTGCATGTGTCCTGCGCATCCCGAG
TCAAAACCAAACCAGCCTAACCTACTCCCAGGGTTCTCTTCACCGCAGCGTCAGCCAGCTCA
TGGACATTCAAGACAAGAAAGTCTTGATGGACAACTCACTGTGGGAAGCCATCATGGGCCA
CAACAGTGGACTGTATGTGGATGAAGAGGACCTGATGAACGCCATCAAGGATTTCAGCTCA
GTGACTAAGGAACGCACCACATTCACAGACACCCACCTGTAAAGGATGGAAACCCAGAAGA
CGTAACCCTGGAATGCAAGGTCTGCACCCATTTCCTCCTGGGTTATCACTCACACATCATAAA
TGCTGAAAAGCCATTGTTTATTATCCTATAATTCTTTAAAGAAATGATGACTGTTTTTGAAAG
TGTTCCTTCCTAATAGAGGTCTAAGAAATGATATTTTTCTCATCTTAAATGAGAGAGAATATT
CATATGAAAATACTTGATTTGCTCTTATTTTGTAGAAGACAAAGAAGTATGTAATTGTCACTT
GGTTCTGTTTGGCAGTGATGCTCCTGGTTAACTGAATAATCAGTGGCAATTTCAAGATGGCT
CACAGTTGTTAGAAGTAGTAAGTTAGTTACTGGCTCAAAAATGATTCTGTTGAAAGGATGTC
ACTGCTGTTCATTTCTATCTGCCATTTCTGTCAGGGTTGACACAATCCTGCAAGAATAGTTAT
TCTAATGATCACAGCTGCTAAATGAATCCCAAACTTTGCACCAGGTCGACAAACTTTTCTGA
AGGTTCTATTTATTTACCATACATAGGGTTACTTACCAAACTTTTTGACAAGGCTGAAGGTTC
TATTTATTTACAATACATAGGGTTACTCACCAAACTTTTTGACAAGGCAACACATAACTTACA
CATAAATGTCTCTGTTCTTGCATTTATGAATTTTCCAAAAATCTAAGGAGTAAACAGCTTATT
TATACATTTTGAGGAGAAAACAAAGTGTTTCACTAGGAACACCTCTACTTGAACCAATGTTT
TTATTTCATATATTTTATAGTTTTGAAACTAGTTTCTCATAAAATTCTGTCAATTCACTGAATA
TCAGAGAATACTGACATCTTCAACCTAGCACATTTCAAATGGAAACTACTGTTCTATTTGCAA
TATTAGGCTGCGTGAAATTTTAAAAGGAAAAATGTATCTGTTCCTTCTAGCATTAACATATAT
ACATGTAGAGACAAGACTATACCTATGTGTATATATATGTATATCATGTATATATTACTCTGC
ACTATATCCCTTCTTTTTGGAGAACTAGCCATTATTTTAGCCACAGAATCAGTAAGAACAGAT
GATATGCAACAGTACCAATTACGGTTCAAAAATGTCTGTCACCTGCTCTAGTTGGATTACAA
AGTCATTGGTGAAAGTCCTATGGCAAGAAAAATTTTCTTGCAAATCATCCACATAAAATCAG
ATATTTAAATTTGTTCTTCATGGAAAACAGAGTAAGAAAACCTCTTGTCTTCCTTCATCCTTA
AAGGTCTTTGTGACCCCAGGAAAATATTGACTCTGTCTAACACACAATAGTCACAATACTTTT
TGTGAATCTACAACCAGAGACAGGCAAAAACTTGTAAAGTAAGGGATAGTCTTACTTATTCT
GCCTGAAAACAATGTATTACCCCAGGGCCCAACAGTAAAAGATTGTGGACTTTTTGGGTATT
GAGATTTCATCTAGCTCTGTGAGAGAGCAGCTCCTCAGACTGACCAACTCCTAGACAAAGTT
TGCCAACCATAAGTGTCAAAAGCACAGGCCAGTATTAAGCAGAAGTTCTACCACCTTATTAG
AACTGCTATAAACAAAAGCATCTGAAATAATTGTGCACATCTGGCAGTGACTGTAGAAAAT
ACGAAATATATATTTCTCGCCAAGTTTTTATACTTTCTGAAATGAAAACATAGGATTGACTAG
TTTACTGGTTTTTATTCCCATATGCCGATTCTGGGACAATAAAGTTGTTTAAAGCTGGCACAA
ATAAGCATTAACCAAGGCTGTGTCCACCTTCTGTGAGCTACTTAAGGTATATAGGAAAGGA
GTGGTCACAAACTTGCATCCTAATCCTTGGTGGACTCTTCTAAGAATACAGTTTGCTAGTCAC
AAAGAATAGTCTACAAATATGCTTTGCTAGGTTCAGAAGATTGAGTTTATCCTGATTTTTGA
AAAATTAACCAGGTATCTTTATCACTGTGTATTTTTCCAAGCACAGTATAAAATTTTAACAAC
GCACAAAAAAATACAGAACTGCAGGGGATTTTATCTTGGATCATTATCCATTTAATCATCTA
ATTAGACATGAACTCAGTTAGCTGAATCATTTACATTTTGACTCCATAGCTTAGGGCAGACA
GAAGCCTGTATGGCTTCTGCCCAGAACTCTGTCCCCTGCTACATGTCTAAGTTTACTTGTATT
TATTTCAGAGAAGAACTCTAAGATGTTGCTTTGCTACTTTAAGTGGTATTGCGTGCCAAGCC
TCTATTATACAAACCATGCAGACTCGCCTCTAGAGATTCTGATTCGGTTGATCTGGGGTGTG
TGGCTGAGGCATCAGTACTTTTTAAAGCTTCCAGGTGTTCTAATGTTGAGACCCACTGATGT
TCCACAATCTGGAAGAAATCATGTACAGGAATAATATGCTATGCACAGGGACTATGCTCCTT
GGCTCACCCCTTCTCCCTTATAAACAATGAGCAGTTCTTGATGAACCTCTTTAAATTTAAATC
TCCTGACTCACATTTTACCAATTGTACATGCCACATTCTCAGCTTACGAACTACCATGTTTTGT
TATTCTTAATATCAACTGTTTGGTAAGAGTACAGTTGTTTTTATACACTCTAAGAAATGTGTT
TATAATCTACTGTAATTTCCACTAAATGGAACCCAAATATTAATGTTATGGTACCATATACTG
ATGTAAAAATCATGCTGGCATCCATGAACACACCGGTAAATAAAACATAGTCCAAGTGGAA
GAATTCATTAATAAGGAACTTTTAATTATGTCACAAATGAATAGTTGGTTTCCAATGCACAA
ATATCATGTAAACTAATCTAAAGATGGTTTGCTTAATAAATATTTGAATGTGACC

Translation of this mRNA is expected to result in expression of human Usherin protein lacking part of laminin EGF-like domain 4, all of domains 5, 6 and 7 and part of domain 8:
(Partial domain 4 in bold, partial domain 8 double underlined)

(SEQ ID NO: 2)
MNCPVLSLGSGFLFQVIEMLIFAYFASISLTESRGLFPRLENVGAFKKVSIVPTQAVCGLPDRSTFC
HSSAAAESIQFCTQRFCIQDCPYRSSHPTYTALFSAGLSSCITPDKNDLHPNAHSNSASFIFGNHK
SCFSSPPSPKLMASFTLAVWLKPEQQGVMCVIEKTVDGQIVFKLTISEKETMFYYRTVNGLQPPI
KVMTLGRILVKKWIHLSVQVHQTKISFFINGVEKDHTPFNARTLSGSITDFASGTVQIGQSLNGLE
QFVGRMQDFRLYQVALTNREILEVFSGDLLRLHAQSHCRCPGSHPRVHPLAQRYCIPNDAGDT
ADNRVSRLNPEAHPLSFVNDNDVGTSWVSNVFTNITQLNQGVTISVDLENGQYQVFYIIIQFFS
PQPTEIRIQRKKENSLDWEDWQYFARNCGAFGMKNNGDLEKPDSVNCLQLSNFTPYSRGNVT
FSILTPGPNYRPGYNNFYNTPSLQEFVKATQIRFHFHGQYYTTETAVNLRHRYYAVDEITISGRCQ
CHGHADNCDTTSQPYRCLCSQESFTEGLHCDRCLPLYNDKPFRQGDQVYAFNCKPCQCNSHSK
SCHYNISVDPFPFEHFRGGGGVCDDCEHNTTGRNCELCKDYFFRQVGADPSAIDVCKPCDCDT
VGTRNGSILCDQIGGQCNCKRHVSGRQCNQCQNGFYNLQELDPDGCSPCNCNTSGTVDGDIT
CHQNSGQCKCKANVIGFYISPGNATGCLPCSCHTTGAVNHICNSLTGQCVCQDASIAGQRCDQ
CKDHYFGFDPQTGRCQPCNCHLSGALNETCHLVTGQCFCKQFVTGSKCDACVPSASHLDVNNL
LGCSKTPFQQPPPRGQVQSSSAINLSWSPPDSPNAHWLTYSLLRDGFEIYTTEDQYPYSIQYFLD
TDLLPYTKYSYYIETTNVHGSTRSVAVTYKTKPGVPEGNLTLSYIIPIGSDSVTLTWTTLSNQSGPIE
KYILSCAPLAGGQPCVSYEGHETSATIWNLVPFAKYDFSVQACTSGGCLHSLPITVTTAQAPPQR
LSPPKMQKISSTELHVEWSPPAELNGIIIRYELYMRRLRSTKETTSEESRVFQSSGWLSPHSFVESA
NENALKPPQTMTTITGLEPYTKYEFRVLAVNMAGSVSSAWVSERTGESAPVFMIPPSVFPLSSYS
LNISWEKPADNVTRGKVVGYDINMLSEQSPQQSIPMAFSQLLHTAKSQELSYTVEGLKPYRIYEF
TITLCNSVGCVTSASGAGQTLAAAPAQLRPPLVKGINSTTIHLKWFPPEELNGPSPIYQLERRESSL
PALMTTMMKGIRFIGNGYCKFPSSTHPVNTDFTGIKASFRTKVPEGLIVFAASPGNQEEYFALQL
KKGRLYFLFDPQGSPVEVTTTNDHGKQYSDGKWHEIIAIRHQAFGQITLDGIYTGSSAILNGSTVI
GDNTGVFLGGLPRSYTILRKDPEIIQKGFVGCLKDVHFMKNYNPSAIWEPLDWQSSEEQINVYN
SWEGCPASLNEGAQFLGAGFLELHPYMFHGGMNFEISFKFRTDQLNGLLLFVYNKDGPDFLAM
ELKSGILTFRLNTSLAFTQVDLLLGLSYCNGKWNKVIIKKEGSFISASVNGLMKHASESGDQPLVV
NSPVYVGGIPQELLNSYQHLCLEQGFGGCMKDVKFTRGAVVNLASVSSGAVRVNLDGCLSTDS
AVNCRGNDSILVYQGKEQSVYEGGLQPFTEYLYRVIASHEGGSVYSDWSRGRTTGAAPQSVPTP
SRVRSLNGYSIEVTWDEPVVRGVIEKYILKAYSEDSTRPPRMPSASAEFVNTSNLTGILTGLLPFKN
YAVTLTACTLAGCTESSHALNISTPQEAPQEVQPPVAKSLPSSLLLSWNPPKKANGIITQYCLYMD
GRLIYSGSEENYTVTDLAVFTPHQFLLSACTHVGCTNSSWVLLYTAQLPPEHVDSPVLTVLDSRTI
HIQWKQPRKISGILERYVLYMSNHTHDFTIWSVIYNSTELFQDHMLQYVLPGNKYLIKLGACTGG
GCTVSEASEALTDEDIPEGVPAPKAHSYSPDSFNVSWTEPEYPNGVITSYGLYLDGILIHNSSELSY
RAYGFAPWSLHSFRVQACTAKGCALGPLVENRTLEAPPEGTVNVFVKTQGSRKAHVRWEAPFR
PNGLLTHSVLFTGIFYVDPVGNNYTLLNVTKVMYSGEETNLWVLIDGLVPFTNYTVQVNISNSQ
GSLITDPITIAMPPGAPDGVLPPRLSSATPTSLQVVWSTPARNNAPGSPRYQLQMRSGDSTHGF
LELFSNPSASLSYEVSDLQPYTEYMFRLVASNGFGSAHSSWIPFMTAEDKPGPVVPPILLDVKSR
MMLVTWQHPRKSNGVITHYNIYLHGRLYLRTPGNVTNCTVMHLHPYTAYKFQVEACTSKGCSL
SPESQTVWTLPGAPEGIPSPELFSDTPTSVIISWQPPTHPNGLVENFTIERRVKGKEEVTTLVTLPR
SHSMRFIDKTSALSPWTKYEYRVLMSTLHGGTNSSAWVEVTTRPSRPAGVQPPVVTVLEPDAV
QVTWKPPLIQNGDILSYEIHMPDPHITLTNVTSAVLSQKVTHLIPFTNYSVTIVACSGGNGYLGG
CTESLPTYVTTHPTVPQNVGPLSVIPLSESYVVISWQPPSKPNGPNLRYELLRRKIQQPLASNPPE
DLNRWHNIYSGTQWLYEDKGLSRFTTYEYMLFVHNSVGFTPSREVIVTTLAGLPERGANLTASV
LNHTAIDVRWAKPTVQDLQGEVEYYTLFWSSATSNDSLKILPDVNSHVIGHLKPNTEYWIFISVF
NGVHSINSAGLHATTCDGEPQGMLPPEVVIINSTAVRVIWTSPSNPNGVVTEYSIYVNNKLYKT
GMNVPGSFILRDLSPFTIYDIQVEVCTIYACVKSNGTQITTVEDTPSDIPTPTIRGITSRSLQIDWVS
PRKPNGIILGYDLLWKTWYPCAKTQKLVQDQSDELCKAVRCQKPESICGHICYSSEAKVCCNGVL
YNPKPGHRCCEEKYIPFVLNSTGVCCGGRIQEAQPNHQCCSGYYARILPGEVCCPDEQHNRVSV
GIGDSCCGRMPYSTSGNQICCAGRLHDGHGQKCCGRQIVSNDLECCGGEEGVVYNRLPGMFC
CGQDYVNMSDTICCSASSGESKAHIKKNDPVPVKCCETELIPKSQKCCNGVGYNPLKYVCSDKIS
TGMMMKETKECRILCPASMEATEHCGRCDFNFTSHICTVIRGSHNSTGKASIEEMCSSAEETIHT
GSVNTYSYTDVNLKPYMTYEYRISAWNSYGRGLSKAVRARTKEDVPQGVSPPTWTKIDNLEDTI
VLNWRKPIQSNGPIIYYILLRNGIERFRGTSLSFSDKEGIQPFQEYSYQLKACTVAGCATSSKVVAA
TTQGVPESILPPSITALSAVALHLSWSVPEKSNGVIKEYQIRQVGKGLIHTDTTDRRQHTVTGLQP
YTNYSFTLTACTSAGCTSSEPFLGQTLQAAPEGVWVTPRHIIINSTTVELYWSLPEKPNGLVSQYQ
LSRNGNLLFLGGSEEQNFTDKNLEPNSRYTYKLEVKTGGGSSASDDYIVQTPMSTPEEIYPPYNIT
VIGPYSIFVAWIPPGILIPEIPVEYNVLLNDGSVTPLAFSVGHHQSTLLENLTPFTQYEIRIQACQNG
SCGVSSRMFVKTPEAAPMDLNSPVLKALGSACIEIKWMPPEKPNGIIINYFIYRRPAGIEEESVLF
VWSEGALEFMDEGDTLRPFTLYEYRVRACNSKGSVESLWSLTQTLEAPPQDFPAPWAQATSAH
SVLLNWTKPESPNGIISHYRVVYQERPDDPTFNSPTVHAFTVKGTSHQAHLYGLEPFTTYRIGVV
AANHAGEILSPWTLIQTLESSPSGLRNFIVEQKENGRALLLQWSEPMRTNGVIKTYNIFSDGFLEY
SGLNRQFLFRRLDPFTLYTLTLEACTRAGCAHSAPQPLWIDEAPPDSQLAPTVHSVKSTSVELSW
SEPVNPNGKIIRYEVIRRCFEGKAWGNQTIQADEKIVFTEYNTERNTFMYNDTGLQPWTQCEYK
IYTWNSAGHTCSSWNVVRTLQAPPEGLSPPVISYVSMNPQKLLISWIPPEQSNGIIQSYRLQRNE
MLYPFSFDPVTFNYTDEELLPFSTYSYALQACTSGGCSTSKPTSITTLEAAPSEVSPPDLWAVSAT
QMNVCWSPPTVQNGKITKYLVRYDNKESLAGQGLCLLVSHLQPYSQYNFSLVACINGGCTASV
SKSAWTMEALPENMDSPTLQVTGSESIEITWKPPRNPNGQIRSYELRRDGTIVYTGLETRYRDFT
LTPGVEYSYTVTASNSQGGILSPLVKDRTSPSAPSGMEPPKLQARGPQEILVNWDPPVRINGDII
NYTLFIRELFERETKIIHINTTHNSFGMQSYIVNQLKPFHRYEIRIQACTTLGCASSDWTFIQTPEIA
PLMQPPPHLEVQMAPGGFQPTVSLLWTGPLQPNGKVLYYELYRRQIATQPRKSNPVLIYNGSS
TSFIDSELLPFTEYEYQVWAVNSAGKAPSSWTWCRTGPAPPEGLRAPTFHVISSTQAVVNISAP
GKPNGIVSLYRLFSSSAHGAETVLSEGMATQQTLHGLQAFTNYSIGVEACTCFNCCSKGPTAELR
THPAPPSGLSSPQIGTLASRTASFRWSPPMFPNGVIHSYELQFHVACPPDSALPCTPSQIETKYT
GLGQKASLGGLQPYTTYKLRVVAHNEVGSTASEWISFTTQKELPQYRAPFSVDSNLSVVCVNWS
DTFLLNGQLKEYVLTDGGRRVYSGLDTTLYIPRTADKTFFFQVICTTDEGSVKTPLIQYDTSTGLGL
VLTTPGKKKGSRSKSTEFYSELWFIVLMAMLGLILLAIFLSLILQRKIHKEPYIRERPPLVPLQKRMS
PLNVYPPGENHMGLADTKIPRSGTPVSIRSNRSACVLRIPSQNQTSLTYSQGSLHRSVSQLMDIQ
DKKVLMDNSLWEAIMGHNSGLYVDEEDLMNAIKDFSSVTKERTTFTDTHL

### REFERENCES

1. Hartong, D. T., E. L. Berson, and T. P. Dryja, Retinitis pigmentosa. Lancet, 2006. 368(9549): p. 1795-809.
2. Saihan, Z., et al., Update on Usher syndrome. Curr Opin Neurol, 2009. 22(1): p. 19-27.
3. Lentz, J. and B. Keats, Usher Syndrome Type II, in GeneReviews®, R. A. Pagon, et al., Editors. 1993, University of Washington, Seattle: Seattle WA.
4. Sandberg, M. A., et al., Disease course in patients with autosomal recessive retinitis pigmentosa due to the USH2A gene. Invest Ophthalmol Vis Sci, 2008. 49(12): p. 5532-9.
5. Maguire, A. M., et al., Safety and efficacy of gene transfer for Leber's congenital amaurosis. The New England journal of medicine, 2008. 358(21): p. 2240-8.
6. Bainbridge, J. W., et al., Effect of gene therapy on visual function in Leber's congenital amaurosis. N. Engl. J. Med., 2008. 358(21): p. 2231-2239.
7. Cideciyan, A. V., et al., Human gene therapy for RPE65 isomerase deficiency activates the retinoid cycle of vision but with slow rod kinetics. Proc. Nat. Acad. Sci., 2008. 105(39): p. 15112-7.
8. Maguire, A. M., et al., Age-dependent effects of RPE65 gene therapy for Leber's congenital amaurosis: a phase 1 dose-escalation trial. Lancet, 2009. 374(9701): p. 1597-605.
9. Jacobson, S. G., et al., Gene therapy for leber congenital amaurosis caused by RPE65 mutations: safety and efficacy in 15 children and adults followed up to 3 years. Archives of Ophthalmology, 2012. 130(1): p. 9-24.
10. Bennett, J., et al., AAV2 gene therapy readministration in three adults with congenital blindness. Science translational medicine, 2012. 4(120): p. 120ra15.
11. Bowles, D. E., et al., Phase 1 gene therapy for Duchenne muscular dystrophy using a translational optimized AAV vector. Molecular therapy: the journal of the American Society of Gene Therapy, 2012. 20(2): p. 443-55.
12. Maclachlan, T. K., et al., Preclinical safety evaluation of AAV2 -sFLT01—a gene therapy for age-related macular degeneration. Molecular therapy: the journal of the American Society of Gene Therapy, 2011. 19(2): p. 326-34.
13. Nathwani, A. C., et al., Adenovirus-associated virus vector-mediated gene transfer in hemophilia B. The New England journal of medicine, 2011. 365(25): p. 2357-65.
14. Ran, F. A., et al., In vivo genome editing using Staphylococcus aureus Cas9. Nature, 2015. 520(7546): p. 186-91.
15. Swiech, L., et al., In vivo interrogation of gene function in the mammalian brain using CRISPR-Cas9. Nat Biotechnol, 2015. 33(1): p. 102-6.
16. van Wijk, E., et al., Identification of 51 novel exons of the Usher syndrome type 2A (USH2A) gene that encode multiple conserved functional domains and that are mutated in patients with Usher syndrome type II. Am J Hum Genet, 2004. 74(4): p. 738-44.
17. Grati, M., et al., Localization of PDZD7 to the stereocilia ankle-link associates this scaffolding protein with the Usher syndrome protein network. J Neurosci, 2012. 32(41): p. 14288-93.
18. Liu, X., et al., Usherin is required for maintenance of retinal photoreceptors and normal development of cochlear hair cells. Proc Natl Acad Sci U S A, 2007. 104(11): p. 4413-8.
19. Aller, E., et al., The USH2A c.2299delG mutation: dating its common origin in a Southern European population. Eur J Hum Genet, 2010. 18(7): p. 788-93.
20. Yan, D., et al., Mutation analysis in the long isoform of USH2A in American patients with Usher Syndrome type II. J Hum Genet, 2009. 54(12): p. 732-8.
21. Kemaladewi, D. U. and R. D. Cohn, Exon Snipping in Duchenne Muscular Dystrophy. Trends Mol Med, 2016. 22(3): p. 187-9.
22. Long, C., et al., Postnatal genome editing partially restores dystrophin expression in a mouse model of muscular dystrophy. Science, 2016. 351(6271): p. 400-3.
23. Nelson, C. E., et al., In vivo genome editing improves muscle function in a mouse model of Duchenne muscular dystrophy. Science, 2016. 351(6271): p. 403-7.
24. Tabebordbar, M., et al., In vivo gene editing in dystrophic mouse muscle and muscle stem cells. Science, 2016. 351(6271): p. 407-11.
25. Young, C. S., et al., A Single CRISPR-Cas9 Deletion Strategy that Targets the Majority of DMD Patients Restores Dystrophin Function in hiPSC-Derived Muscle Cells. Cell Stem Cell, 2016. 18(4): p. 533-40.
26. Kalinec, F., et al., Establishment and characterization of conditionally immortalized organ of corti cell lines. Cell Biol Int, 1999. 23(3): p. 175-84.
27. Millan, J. M., et al., An update on the genetics of usher syndrome. J Ophthalmol, 2011. 2011: p. 417217.
28. Lenassi, E., et al., A detailed clinical and molecular survey of subjects with nonsyndromic USH2A retinopathy reveals an allelic hierarchy of disease-causing variants. Eur J Hum Genet, 2015. 23(10): p. 1318-27.
29. Suzuki, K., et al., In vivo genome editing via CRISPR/Cas9 mediated homology-independent targeted integration. Nature, 2016. 540(7631): p. 144-149.
30. Zou, J., et al., Whirlin replacement restores the formation of the USH2 protein complex in whirlin knockout photoreceptors. Invest Ophthalmol Vis Sci, 2011. 52(5): p. 2343-51.
31. Zou, J., et al., Deletion of PDZD7 disrupts the Usher syndrome type 2 protein complex in cochlear hair cells and causes hearing loss in mice. Hum Mol Genet, 2014. 23(9): p. 2374-90.
32. Davis, E. E., et al., TTC21B contributes both causal and modifying alleles across the ciliopathy spectrum. Nat Genet, 2011. 43(3): p. 189-96.
33. Zhang, Q., et al., Knockdown of ttc26 disrupts ciliogenesis of the photoreceptor cells and the pronephros in zebrafish. Mol Biol Cell, 2012. 23(16): p. 3069-78.
34. Wang, H., et al., One-step generation of mice carrying mutations in multiple genes by CRISPR/Cas-mediated genome engineering. Cell, 2013. 153(4): p. 910-8.
35. Inui, M., et al., Rapid generation of mouse models with defined point mutations by the CRISPR/Cas9 system. Sci Rep, 2014. 4: p. 5396.
36. Mashiko, D., et al., Feasibility for a large scale mouse mutagenesis by injecting CRISPR/Cas plasmid into zygotes. Dev Growth Differ, 2014. 56(1): p. 122-9.
37. Lu, B., et al., Cell transplantation to arrest early changes in an ush2a animal model. Invest Ophthalmol Vis Sci, 2010. 51(4): p. 2269-76.
38. Liu, Q., J. Zuo, and E. A. Pierce, The retinitis pigmentosa 1 protein is a photoreceptor microtubule-associated protein. J Neurosci, 2004. 24(29): p. 6427-36.
39. Cheng, A. W., et al., Multiplexed activation of endogenous genes by CRISPR-on, an RNA-guided transcriptional activator system. Cell Res, 2013. 23(10): p. 1163-71.
40. Ousterout, D. G., et al., Multiplex CRISPR/Cas9-based genome editing for correction of dystrophin mutations that cause Duchenne muscular dystrophy. Nat Commun, 2015. 6: p. 6244.
41. Platt, R. J., et al., CRISPR-Cas9 knockin mice for genome editing and cancer modeling. Cell, 2014. 159(2): p. 440-55.
42. Senis, E., et al., CRISPR/Cas9-mediated genome engineering: an adeno-associated viral (AAV) vector toolbox. Biotechnol J, 2014. 9(11): p. 1402-12.
43. Tsai, S. Q., et al., GUIDE-seq enables genome-wide profiling of off-target cleavage by CRISPR-Cas nucleases. Nat Biotechnol, 2015. 33(2): p. 187-97.
44. Tsai, S. Q., et al., Open-source guideseq software for analysis of GUIDE-seq data. Nat Biotechnol, 2016. 34(5): p. 483.
45. Matsuda, T. and C. L. Cepko, Controlled expression of transgenes introduced by in vivo electroporation. Proc Natl Acad Sci USA, 2007. 104(3): p. 1027-32.
46. Jelcick, A. S., et al., Genetic variations strongly influence phenotypic outcome in the mouse retina. PLoS One, 2011. 6(7): p. e21858.
47. Liu, Q., A. Saveliev, and E. A. Pierce, The severity of retinal degeneration in Rplh gene-targeted mice is dependent on genetic background. Invest Ophthalmol Vis Sci, 2009. 50(4): p. 1566-74.
48. Vijayakumar S, Depreux FF, Jodelka FM, et al. Rescue of peripheral vestibular function in Usher syndrome mice using a splice-switching antisense oligonucleotide. Hum Mol Genet. 2017 Sep. 15; 26(18):3482-3494.
49. Slijkerman RW, Vach6 C, Dona M, et al. Antisense Oligonucleotide-based Splice Correction for USH2A-associated Retinal Degeneration Caused by a Frequent Deep-intronic Mutation. Mol Ther Nucleic Acids. 2016 Nov. 1; 5(10):e381.
50. Annemieke Aartsma-Rus, et al. Theoretic applicability of antisense-mediated exon skipping for Duchenne muscular dystrophy. Hum Mutat. 2009 March;30 (3):293-9.
51. B6roud C, et al. Multiexon skipping leading to an artificial DMD protein lacking amino acids from exons 45 through 55 could rescue up to 63% of patients with Duchenne muscular dystrophy. Hum Mutat. 2007 February;28(2):196-202.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid comprising sequences encoding a Cas9 protein, and a first gRNA, and a second gRNA, wherein the first and second gRNAs are targeted to sequences flanking exon 13 of a human usherin (USH2A) gene, wherein the target sequence of the first gRNA is in the 3′ 1500 base pairs (bp) of intron 12 of the USH2A gene, and the target sequence of the second gRNA is in the 5′ 1500 bp of intron 13 of the USH2A gene.

2. The nucleic acid of claim 1, wherein the first gRNA comprises a target sequence shown in Table 1 or 6a, and/or wherein the second gRNA comprises a target sequence shown in Table 2 or 6b.

3. A nucleic acid comprising sequences encoding a CRISPR Cas protein, and a gRNA targeted to a splice acceptor site for exon 13 of a human USH2A gene.

4. The nucleic acid of claim 3, wherein the gRNA comprises a target sequence shown in Table 3.

5. The nucleic acid of claim 3, wherein the nucleic acid encodes a CRISPR Cas protein selected from S. aureus Cas9, Acidaminococcus sp. BV3L6 Cpf1 (AsCpf1), orLachnospiraceae bacterium ND2006 Cpf1 (LbCpf1).

6. The nucleic acid of claim 5, wherein the Cas9 CRISPR Cas protein comprises a nuclear localization signal, optionally a C-terminal nuclear localization signal and/or an N-terminal nuclear localization signal; and/or wherein the sequences encoding the CRISPR Cas protein comprises a polyadenylation signal.

7. (canceled)

8. The nucleic acid of claim 1, which comprises a viral delivery vector further comprising a promoter for driving expression of the sequences encoding the CRISPR Cas protein, preferably a CMV, EFS, or hGRK1 promoter.

9. (canceled)

10. The nucleic acid of claim 8, wherein the viral delivery vector comprises an adeno-associated virus (AAV) vector.

11. The nucleic acid of claim 8, which comprises:
(i) a first guide RNA comprising a targeting domain sequence selected from the group listed in Table 1 or 6a and a second guide RNA comprising a targeting domain sequence selected from the group listed in Table 2 or 6b, or a single guide RNA comprising a targeting domain sequence selected from the group listed in Table 3;
(ii) a first and a second inverted terminal repeat sequence (ITR); and
(iii) the promoter selected from the group consisting of a CMV, an EFS, or an hGRK1 promoter.

12. (canceled)

13. (canceled)

14. (canceled)

15. (canceled)

16. (canceled)

17. (canceled)

18. (canceled)

19. (canceled)

20. An isolated cell, wherein the genome of the cell comprises a human USH2A gene lacking exon 13 or a mutation in a splice acceptor site for exon 13 of the USH2A gene, and wherein the cells express a human usherin protein lacking exon 13, and wherein the cells do not express a functional mouse usherin protein.

21. The isolated cell of claim 20, wherein the cell is a cultured mouse cochlear cell.

22. The isolated cell of claim 20, wherein the cultured mouse cochlear cell is an Oc-K1 cell.

23. (canceled)

24. (canceled)

25. A method of altering the genome of a cell, the method comprising using CRISPR editing to target an USH2A gene comprising a wild type splice acceptor to destroy the exon 13 splice acceptor.

26. (canceled)

27. (canceled)

28. (canceled)

29. (canceled)

30. (canceled)

31. (canceled)

32. (canceled)

33. (canceled)

34. (canceled)

35. The method of claim 25, wherein the cell is from a subject suffering from Usher syndrome type 2A.

36. The method of claim 25, wherein the cell is a retinal cell or a photoreceptor cell.

37. The method of claim 36, wherein the photoreceptor cell is a cone photoreceptor cell or a cone cell, a rod photoreceptor cell or a rod cell or a macular cone photoreceptor cell.

38. The method of claim 25, comprising contacting the cell with a recombinant viral particle comprising:
a nucleotide sequence encoding a gRNA molecule comprising a targeting domain sequence targeted to a splice acceptor site for exon 13 of an USH2A gene, optionally a gRNA selected from Table 3;
a nucleotide sequence encoding a CRISPR Cas molecule;
wherein said viral particle is capable of delivery to a non-dividing cell, and wherein said contacting results in removal of exon 13 of human USH2A gene.

39. The method of claim 38, wherein the viral particle is an adeno-associated virus (AAV) viral particle.

40. (canceled)

41. The method of claim 38, wherein the CRISPR Cas molecule is SaCas9-KKH and the gRNA comprises one of SEQ ID NOs:143-155, 157-169, or 171-183, or the CRISPR Cas molecule is SaCas9 and the gRNA comprises one of SEQ ID NOs:156, 170, or 184.

42. The method of claim 38, wherein the CRISPR Cas molecule is AsCpf1 RR and the gRNA comprises SEQ ID NO:48; the CRISPR Cas molecule is AsCpf1 RVR and the gRNA comprises SEQ ID NO:46; or the CRISPR Cas molecule is RRR LbCpf1 and the gRNA comprises SEQ ID NO:47.


SEQUENCE LISTING (189 sequences)

SEQ ID NO: 1 - DNA, 18241 bp, synthetic construct
  source 1..18241: mol_type = other DNA
tgtttgctctgcagaatactttacctgggcacccaagtcttccttccagcattcctgctgctacagcctatttgctgagtaaccaggggttacagcagcgttgccaggcaacgagggacagcggtcctgttgaagagccatttgtcacactgaggggactggttgaaatgcaataaagaaatgataccagcagctactcatgtcttcgccattgctaagaacgtcgttggtattaccttactctgagaacgtgtctgcagtttccagaaaatggagtatcgcaacatcacttaaagtaccctgcttcaaagtattgctggcaagtggcgtgggcctgattatttatttagaaatgctttatcaggaggagaatgcttttttgtaaacatgaattgcccagttctttcattgggctctggcttcttgtttcaggtcattgaaatgttgatctttgcctattttgcttcaatatccttgactgagtcacgaggtcttttcccaaggctggagaacgtgggagctttcaagaaagtttccatcgtgccaacccaagcagtatgtggactcccagaccgaagcactttttgtcacagctctgctgctgctgaaagtattcagttctgtacccagcggttttgtattcaggattgcccatacagatcttcacaccctacctacactgcccttttctcagcaggcctcagtagctgcatcacaccagacaagaatgatctgcatcctaacgcccatagcaattctgcaagttttatttttggaaatcacaagagctgcttttcttctcctccttctccaaagctgatggcatcatttaccttagctgtatggctgaaacctgagcaacaaggtgtaatgtgtgttatagaaaagacagtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagttcgtaaaagccacgcaaataaggtttcattttcatgggcagtactatacaactgagactgctgttaacctcagacacagatattatgcagtggacgaaatcaccattagtgggagatgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgtaaaccttgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgcaaaccctgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgcagtccctgtaactgcaatacctctgggacagtggatggagatattacctgtcaccaaaattcaggccagtgcaagtgcaaagcaaacgttattggtttttatatttctccaggcaatgccactggctgcctgccatgctcatgccatacaactggtgcagttaatcacatctgtaatagcctgactggtcagtgtgtttgccaagatgcttccattgctgggcaacgttgtgaccaatgcaaagaccattactttggatttgatcctcagactggaagatgtcagccttgtaattgtcatctctcaggagccttgaatgaaacctgtcacttggtcacaggccagtgtttctgtaaacaatttgtcactggctcaaagtgtgatgcttgtgttcccagtgcaagccacttggatgtcaacaatctattgggttgcagcaaaactccattccagcaacctccgcccagaggacaagttcaaagttcttctgctatcaatctctcctggagtccacctgattctccaaatgcccactggcttacttacagtttactcagggatggttttgaaatctacacaacagaggatcaatacccatacagtattcaatacttcttagacacagacctgttaccatataccaaatattcctattacattgagaccaccaatgtgcatggttcaacaaggagtgtagctgtcacttacaagacaaaaccaggggtcccagagggaaacttgactttaagttatatcattcctattggctcagactctgtgacacttacctggacaacactctcaaatcaatctggtcccatagagaaatatattttgtcctgtgcccctttggctggtggtcagccatgtgtttcctacgaaggtcatgaaacctcagctaccatctggaatctggttccatttgccaagtacgatttttctgtacaggcgtgtactagcgggggctgtttacacagcttgcccattacagtgaccacagcccaggcccctccccaaagactaagtccacctaagatgcagaaaatcagttctacagaacttcatgtagaatggtctccaccagcggaactaaatggaataattataagatatgaactatacatgagaagactgagatctactaaagaaaccacatctgaggaaagtcgagtttttcagagcagtggttggctcagtcctcattcatttgtagaatcggccaatgaaaatgcattaaaacctcctcaaacaatgacaaccatcactggcttggagccatacaccaagtatgagttcagagtcttagctgtgaatatggctggaagtgtgtcttctgcctgggtctcagaaagaacgggagaatcagcacctgtattcatgatccctccttcagtctttcccctctcttcgtactctctcaatatctcctgggagaagccagcagataatgttacaagaggaaaagttgtggggtatgacatcaatatgctttctgaacaatcacctcaacagtctattcccatggcgttttcacagctgttgcacactgctaaatcccaagaactatcttacactgtagaaggactgaaaccttataggatatatgagtttactattactctctgcaattcagttggttgtgtgaccagtgcttcgggagcaggacaaactttagcagcagcaccagcacaactgaggccacctctggttaaaggaatcaacagcacaacaatccatcttaagtggtttccacctgaagaactgaatggaccctctcctatatatcagctggaaaggagagagtcatctctaccagctctgatgaccacgatgatgaaaggaatccgtttcataggaaatgggtattgtaaatttcccagctccactcacccagtcaatacagacttcactggcattaaggccagctttcgaacaaaagtgcctgaaggtttgattgtctttgcagcatcacctggcaatcaggaagagtattttgcacttcagttgaagaagggacgtctttattttctttttgatcctcaggggtcaccagtggaagtaactacaactaatgatcatggcaaacaatatagtgatggaaaatggcatgaaataattgctattaggcatcaggcttttggccaaatcactctggatgggatatatacaggttcctctgccatcctgaatggtagtactgttattggagataacacaggagtctttctgggagggctcccgcgaagttataccatcctcaggaaggatcctgagataatccaaaaaggttttgtgggctgtctcaaggatgtacattttatgaagaattacaatccgtcagctatttgggaacctctggattggcagagttctgaagaacaaatcaacgtgtataacagctgggagggatgtcccgcttcattaaatgagggagctcagttcctaggagcagggttcctggaacttcatccatatatgtttcatggtggaatgaactttgagatttcctttaagttcagaactgaccaattaaatggattgcttcttttcgtttataacaaagatggacctgattttcttgctatggagctgaaaagtggaatattgaccttccggttaaataccagtcttgcctttacacaagtggatctattgctggggctatcctattgtaatggaaagtggaataaagtcattattaaaaaggaaggctctttcatatcagcaagtgtgaatggactgatgaagcatgcatcggagtccggagaccagccactggtggtgaattcaccagtttatgtgggaggaatcccacaggaactgctgaactcttatcaacatttgtgtttggaacaaggtttcggtggttgcatgaaggatgttaaatttacacggggtgctgtcgttaacttggcatctgtgtccagcggtgctgtcagagtcaatctggatggatgcctatcaactgacagtgctgttaactgcaggggaaatgactccatcctggtttaccagggaaaagagcagagtgtttacgagggtggtctccagccttttacagaatacctgtatcgagtgatagcctcgcatgaaggaggttcagtatatagtgattggagtcgaggacgtacaacaggagcagctccacaaagtgtgccaactccctcaagagtccgcagcttaaatggatacagcattgaggtgacctgggatgaacctgttgtcagaggtgtaattgagaagtacattctgaaagcctatagtgaggacagcacccgtccaccccgcatgccctctgccagtgctgaatttgtcaatacaagcaacctcacaggcatattgacaggcttgctacccttcaaaaactatgcagtaaccctaactgcttgcactttggctggctgtactgagagctcacatgcattgaacatctctactccacaagaagccccacaagaggttcagccaccagtagccaaatcccttcccagttctttgctgctctcctggaacccacccaaaaaggcaaatggtattataactcagtactgtttatacatggatgggaggctgatctattcaggcagtgaggagaactacacagtcacagatttagcagtatttacaccccaccagtttctactaagtgcatgcacacatgtgggctgtacaaacagttcctgggtcctactgtacacagcacagctgccaccagaacacgtggattccccagttctgactgtcctggattctagaactatacacatacagtggaaacaaccaagaaaaataagtgggattctggaacgctatgtattatatatgtcaaaccatacacatgattttacaatttggagtgtcatctataacagtacagaacttttccaggatcatatgctacaatacgttttacctggtaataaatatctcatcaagctgggagcttgcacaggtggtgggtgcacagtgagtgaggccagtgaggccctaactgacgaggacatacccgaaggcgtgccagcccccaaagcccactcatattcacctgactcctttaatgtctcctggactgagcctgaatatccgaatggtgttatcacgagttatggattatatctagatggtatattaatccacaattcctcagaactcagctatcgtgcttacggatttgctccttggagtttacattccttcagagtccaagcatgcacggccaaaggttgtgctctgggcccactggtggaaaatcgaactctagaagctcctcctgaaggaacagtaaatgtgtttgtcaaaacacagggatcccggaaagcccacgtgaggtgggaagcaccttttcgccctaatggactcttaacacactcagtccttttcactgggatattctatgtagacccagtaggtaataactacacccttctgaatgtcacaaaagtcatgtacagcggagaagagacaaacctttgggtgctcatcgatgggctggttccttttaccaactatactgtacaagtgaatatttcaaatagccaaggcagcttgataactgatcctataacaattgcaatgcctccaggagctccagatggcgtgctgcctcccaggctttcatctgccactccaaccagtcttcaggttgtctggtctacaccagctcgtaataacgctcctggctctcccagataccaactccagatgaggtctggcgactccacccatggatttctagagttattttccaatccttctgcatcgttaagctatgaagtgagtgatctccaaccgtacacagagtatatgtttcggttggttgcctccaatggatttggcagtgcacatagttcttggattccattcatgaccgcagaggacaaacctggacctgtagttcctccgattcttctggatgtgaagtcaagaatgatgttggtcacctggcagcatcctagaaaatccaatggggttattacccattataacatttatctacatggccgtctatacttgagaactcctggaaatgtcactaattgcacagtgatgcatttacacccatacactgcctataagtttcaggtagaagcctgcacttcaaaaggatgttccctttcaccagagtcccagactgtatggacactcccaggggcaccggaagggatcccaagtccagagctgttctctgatactccaacatctgtgattatatcttggcaaccccctacccaccccaatggcttggtggagaatttcacaattgagagaagagtcaaaggaaaggaagaagttactaccctggtgactctcccgaggagtcattccatgaggtttattgacaagacttctgctcttagcccatggacaaaatatgaatatcgggtactgatgagcactcttcatggaggcacaaacagcagtgcttgggtagaagttaccacaagaccctcacgacctgctggggtgcagccacctgtggtgacagtgctggaacccgatgcagtccaggtcacttggaaacccccactcatccagaacggagacatacttagctatgagattcacatgcctgaccctcacatcactttaaccaatgtgacttccgcagtgttaagtcaaaaagttactcatctgattcctttcactaattattctgtcaccattgttgcttgctcagggggtaatgggtaccttggagggtgcacagagagtttacctacctatgttaccactcaccccaccgtacctcagaatgttggcccattgtctgtgattccactaagtgaatcatatgttgtgatttcttggcaaccaccatccaagccaaatggacctaatttgagatatgagcttctgagacgtaaaatccagcagccacttgcatcaaatcccccagaagatttaaatcggtggcacaatatttattcaggaactcagtggctttatgaagataagggtcttagcaggtttacaacctatgaatatatgctcttcgtacacaacagtgtgggttttacaccgagccgagaagtgactgtgacaacgttagctggtcttccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaacagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcacttcaagcgagccttttctaggtcagacactgcaggcagctcctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagtgctagtgatgattacattgttcaaacacctatgtcaacaccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagcagtaggatgtttgtcaaaacacctgaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtggagagtctgtggtcattaacacaaactctggaagctccacctcaagattttccagctccttgggctcaagccacgagtgctcattcagttctgttgaattggacaaagccagaatctcccaatggcattatctcccattaccgtgtggtctaccaggagagacccgacgatcctacatttaacagccctaccgtgcatgctttcacagtgaagggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaagacatacaacatcttcagtgacgggttcctggagtactctggtttgaatcgtcagtttctcttccgccgcctggatcctttcactctctacacactgaccctggaggcctgcaccagagcaggttgtgcacactcggcgcctcagcctctgtggacagatgaagcccctccagactctcagctggctcctactgtccactctgtgaagtccaccagtgttgagctgagctggtctgagcctgttaacccaaatggaaaaataattcgctatgaagtgattcgcagatgcttcgagggaaaagcttggggaaatcagacgatccaggccgacgagaaaattgttttcacagaatataacactgaaaggaatacatttatgtataatgacacaggtttgcaaccatggacgcagtgtgaatataaaatctacacttggaattcagctgggcatacctgtagctcttggaatgtggtgaggacattgcaagcacctccagaaggtctctctccacctgtgatatcctatgtttctatgaatccccaaaaactgctgatttcctggatcccaccagaacagtctaatggtattatccagtcctataggcttcaaaggaatgaaatgctctatccttttagctttgatcctgtgactttcaattacactgatgaagagcttcttcctttttccacctatagctatgcactccaagcctgcacgagtggaggatgctccaccagcaaacccaccagcatcacaactctggaggctgctccatcagaagtcagccctccagatctttgggccgtcagtgccactcaaatgaatgtatgttggtcaccgcccacagtgcaaaatggaaagattactaaatatttagttagatatgataataaagagtcccttgctggccagggcctgtgcctgctggtttcccacctgcagccttactctcagtataacttctcccttgtagcctgcacgaatggaggttgcacagctagtgtgtcaaaatctgcctggacaatggaggccctgccagagaacatggactctccaacattgcaagtcacaggctcagaatcaatagaaatcacctggaaacctccaagaaacccaaatggccagatcagaagttatgaacttaggagggatggaaccattgtatatacaggcttggaaacacgctatcgtgattttactctcaccccaggtgtggagtatagctacacagtaactgccagcaacagccaagggggtattttgagtcctcttgtcaaagatcgaaccagcccctcagcaccctcagggatggaacctccaaaattgcaggccaggggtcctcaggagatcttagtgaactgggaccctccagtgagaacaaatggtgatatcatcaattataccctcttcatccgtgaactatttgaaagagaaactaaaatcatacacataaacacaactcataattcttttggtatgcagtcatatatagtaaaccagctgaagccatttcacaggtatgaaatacgaattcaagcgtgcaccaccctgggatgtgcatcaagtgactggacattcatacagacccctgagattgcacctttgatgcaaccccctccacatctggaggtacaaatggctccaggaggattccagccaactgtttctcttttgtggacaggaccgctgcagccaaatggaaaagttttgtattacgaattatacagaagacaaatagcaactcagcctagaaaatccaatccagtcctaatctataacggaagctcaacatcttttatagattccgaactattgcctttcacagagtatgagtatcaggtctgggcagtgaattctgcaggaaaagcccccagtagctggacatggtgcagaaccgggccagccccaccagaaggtctcagagcccccacgttccatgtgatctcttctacccaagcagtggtcaacatcagtgcccctgggaagcccaacgggatcgtcagtctctacaggctgttctccagcagcgcccatggggctgagacagtgctatccgaaggcatggccacccagcagactctccatggccttcaagccttcactaactactctattggagtagaggcctgcacctgcttcaactgttgcagcaaaggaccgacagctgaactgagaacccatcctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctgtaaaggatggaaacccagaagacgtaaccctggaatgcaaggtctgcacccatttcctcctgggttatcactcacacatcataaatgctgaaaagccattgtttattatcctataattctttaaagaaatgatgactgtttttgaaagtgttccttcctaatagaggtctaagaaatgatatttttctcatcttaaatgagagagaatattcatatgaaaatacttgatttgctcttattttgtagaagacaaagaagtatgtaattgtcacttggttctgtttggcagtgatgctcctggttaactgaataatcagtggcaatttcaagatggctcacagttgttagaagtagtaagttagttactggctcaaaaatgattctgttgaaaggatgtcactgctgttcatttctatctgccatttctgtcagggttgacacaatcctgcaagaatagttattctaatgatcacagctgctaaatgaatcccaaactttgcaccaggtcgacaaacttttctgaaggttctatttatttaccatacatagggttacttaccaaactttttgacaaggctgaaggttctatttatttacaatacatagggttactcaccaaactttttgacaaggcaacacataacttacacataaatgtctctgttcttgcatttatgaattttccaaaaatctaaggagtaaacagcttatttatacattttgaggagaaaacaaagtgtttcactaggaacacctctacttgaaccaatgtttttatttcatatattttatagttttgaaactagtttctcataaaattctgtcaattcactgaatatcagagaatactgacatcttcaacctagcacatttcaaatggaaactactgttctatttgcaatattaggctgcgtgaaattttaaaaggaaaaatgtatctgttccttctagcattaacatatatacatgtagagacaagactatacctatgtgtatatatatgtatatcatgtatatattactctgcactatatcccttctttttggagaactagccattattttagccacagaatcagtaagaacagatgatatgcaacagtaccaattacggttcaaaaatgtctgtcacctgctctagttggattacaaagtcattggtgaaagtcctatggcaagaaaaattttcttgcaaatcatccacataaaatcagatatttaaatttgttcttcatggaaaacagagtaagaaaacctcttgtcttccttcatccttaaaggtctttgtgaccccaggaaaatattgactctgtctaacacacaatagtcacaatactttttgtgaatctacaaccagagacaggcaaaaacttgtaaagtaagggatagtcttacttattctgcctgaaaacaatgtattaccccagggcccaacagtaaaagattgtggactttttgggtattgagatttcatctagctctgtgagagagcagctcctcagactgaccaactcctagacaaagtttgccaaccataagtgtcaaaagcacaggccagtattaagcagaagttctaccaccttattagaactgctataaacaaaagcatctgaaataattgtgcacatctggcagtgactgtagaaaatacgaaatatatatttctcgccaagtttttatactttctgaaatgaaaacataggattgactagtttactggtttttattcccatatgccgattctgggacaataaagttgtttaaagctggcacaaataagcattaaccaaggctgtgtccaccttctgtgagctacttaaggtatataggaaaggagtggtcacaaacttgcatcctaatccttggtggactcttctaagaatacagtttgctagtcacaaagaatagtctacaaatatgctttgctaggttcagaagattgagtttatcctgatttttgaaaaattaaccaggtatctttatcactgtgtatttttccaagcacagtataaaattttaacaacgcacaaaaaaatacagaactgcaggggattttatcttggatcattatccatttaatcatctaattagacatgaactcagttagctgaatcatttacattttgactccatagcttagggcagacagaagcctgtatggcttctgcccagaactctgtcccctgctacatgtctaagtttacttgtatttatttcagagaagaactctaagatgttgctttgctactttaagtggtattgcgtgccaagcctctattatacaaaccatgcagactcgcctctagagattctgattcggttgatctggggtgtgtggctgaggcatcagtactttttaaagcttccaggtgttctaatgttgagacccactgatgttccacaatctggaagaaatcatgtacaggaataatatgctatgcacagggactatgctccttggctcaccccttctcccttataaacaatgagcagttcttgatgaacctctttaaatttaaatctcctgactcacattttaccaattgtacatgccacattctcagcttacgaactaccatgttttgttattcttaatatcaactgtttggtaagagtacagttgtttttatacactctaagaaatgtgtttataatctactgtaatttccactaaatggaacccaaatattaatgttatggtaccatatactgatgtaaaaatcatgctggcatccatgaacacaccggtaaataaaacatagtccaagtggaagaattcattaataaggaacttttaattatgtcacaaatgaatagttggtttccaatgcacaaatatcatgtaaactaatctaaagatggtttgcttaataaatatttgaatgtgacc

SEQ ID NO: 2 - protein, 4988 aa, synthetic construct
MNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGFYISPGNATGCLPCSCHTTGAVNHICNSLTGQCVCQDASIAGQRCDQCKDHYFGFDPQTGRCQPCNCHLSGALNETCHLVTGQCFCKQFVTGSKCDACVPSASHLDVNNLLGCSKTPFQQPPPRGQVQSSSAINLSWSPPDSPNAHWLTYSLLRDGFEIYTTEDQYPYSIQYFLDTDLLPYTKYSYYIETTNVHGSTRSVAVTYKTKPGVPEGNLTLSYIIPIGSDSVTLTWTTLSNQSGPIEKYILSCAPLAGGQPCVSYEGHETSATIWNLVPFAKYDFSVQACTSGGCLHSLPITVTTAQAPPQRLSPPKMQKISSTELHVEWSPPAELNGIIIRYELYMRRLRSTKETTSEESRVFQSSGWLSPHSFVESANENALKPPQTMTTITGLEPYTKYEFRVLAVNMAGSVSSAWVSERTGESAPVFMIPPSVFPLSSYSLNISWEKPADNVTRGKVVGYDINMLSEQSPQQSIPMAFSQLLHTAKSQELSYTVEGLKPYRIYEFTITLCNSVGCVTSASGAGQTLAAAPAQLRPPLVKGINSTTIHLKWFPPEELNGPSPIYQLERRESSLPALMTTMMKGIRFIGNGYCKFPSSTHPVNTDFTGIKASFRTKVPEGLIVFAASPGNQEEYFALQLKKGRLYFLFDPQGSPVEVTTTNDHGKQYSDGKWHEIIAIRHQAFGQITLDGIYTGSSAILNGSTVIGDNTGVFLGGLPRSYTILRKDPEIIQKGFVGCLKDVHFMKNYNPSAIWEPLDWQSSEEQINVYNSWEGCPASLNEGAQFLGAGFLELHPYMFHGGMNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRGAVVNLASVSSGAVRVNLDGCLSTDSAVNCRGNDSILVYQGKEQSVYEGGLQPFTEYLYRVIASHEGGSVYSDWSRGRTTGAAPQSVPTPSRVRSLNGYSIEVTWDEPVVRGVIEKYILKAYSEDSTRPPRMPSASAEFVNTSNLTGILTGLLPFKNYAVTLTACTLAGCTESSHALNISTPQEAPQEVQPPVAKSLPSSLLLSWNPPKKANGIITQYCLYMDGRLIYSGSEENYTVTDLAVFTPHQFLLSACTHVGCTNSSWVLLYTAQLPPEHVDSPVLTVLDSRTIHIQWKQPRKISGILERYVLYMSNHTHDFTIWSVIYNSTELFQDHMLQYVLPGNKYLIKLGACTGGGCTVSEASEALTDEDIPEGVPAPKAHSYSPDSFNVSWTEPEYPNGVITSYGLYLDGILIHNSSELSYRAYGFAPWSLHSFRVQACTAKGCALGPLVENRTLEAPPEGTVNVFVKTQGSRKAHVRWEAPFRPNGLLTHSVLFTGIFYVDPVGNNYTLLNVTKVMYSGEETNLWVLIDGLVPFTNYTVQVNISNSQGSLITDPITIAMPPGAPDGVLPPRLSSATPTSLQVVWSTPARNNAPGSPRYQLQMRSGDSTHGFLELFSNPSASLSYEVSDLQPYTEYMFRLVASNGFGSAHSSWIPFMTAEDKPGPVVPPILLDVKSRMMLVTWQHPRKSNGVITHYNIYLHGRLYLRTPGNVTNCTVMHLHPYTAYKFQVEACTSKGCSLSPESQTVWTLPGAPEGIPSPELFSDTPTSVIISWQPPTHPNGLVENFTIERRVKGKEEVTTLVTLPRSHSMRFIDKTSALSPWTKYEYRVLMSTLHGGTNSSAWVEVTTRPSRPAGVQPPVVTVLEPDAVQVTWKPPLIQNGDILSYEIHMPDPHITLTNVTSAVLSQKVTHLIPFTNYSVTIVACSGGNGYLGGCTESLPTYVTTHPTVPQNVGPLSVIPLSESYVVISWQPPSKPNGPNLRYELLRRKIQQPLASNPPEDLNRWHNIYSGTQWLYEDKGLSRFTTYEYMLFVHNSVGFTPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWSLTQTLEAPPQDFPAPWAQATSAHSVLLNWTKPESPNGIISHYRVVYQERPDDPTFNSPTVHAFTVKGTSHQAHLYGLEPFTTYRIGVVAANHAGEILSPWTLIQTLESSPSGLRNFIVEQKENGRALLLQWSEPMRTNGVIKTYNIFSDGFLEYSGLNRQFLFRRLDPFTLYTLTLEACTRAGCAHSAPQPLWTDEAPPDSQLAPTVHSVKSTSVELSWSEPVNPNGKIIRYEVIRRCFEGKAWGNQTIQADEKIVFTEYNTERNTFMYNDTGLQPWTQCEYKIYTWNSAGHTCSSWNVVRTLQAPPEGLSPPVISYVSMNPQKLLISWIPPEQSNGIIQSYRLQRNEMLYPFSFDPVTFNYTDEELLPFSTYSYALQACTSGGCSTSKPTSITTLEAAPSEVSPPDLWAVSATQMNVCWSPPTVQNGKITKYLVRYDNKESLAGQGLCLLVSHLQPYSQYNFSLVACTNGGCTASVSKSAWTMEALPENMDSPTLQVTGSESIEITWKPPRNPNGQIRSYELRRDGTIVYTGLETRYRDFTLTPGVEYSYTVTASNSQGGILSPLVKDRTSPSAPSGMEPPKLQARGPQEILVNWDPPVRTNGDIINYTLFIRELFERETKIIHINTTHNSFGMQSYIVNQLKPFHRYEIRIQACTTLGCASSDWTFIQTPEIAPLMQPPPHLEVQMAPGGFQPTVSLLWTGPLQPNGKVLYYELYRRQIATQPRKSNPVLIYNGSSTSFIDSELLPFTEYEYQVWAVNSAGKAPSSWTWCRTGPAPPEGLRAPTFHVISSTQAVVNISAPGKPNGIVSLYRLFSSSAHGAETVLSEGMATQQTLHGLQAFTNYSIGVEACTCFNCCSKGPTAELRTHPAPPSGLSSPQIGTLASRTASFRWSPPMFPNGVIHSYELQFHVACPPDSALPCTPSQIETKYTGLGQKASLGGLQPYTTYKLRVVAHNEVGSTASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 3 - DNA, 22 bp, synthetic construct
  source 1..22: mol_type = other DNA
ggaatgcagtactgctgaacgg

SEQ ID NO: 4 - DNA, 62 bp, synthetic construct
  source 1..62: mol_type = other DNA
tgcaccattgggaatgcggaggggtggacacgggggtggctacctggacatcggcaatgtgg

SEQ ID NO: 5 - DNA, 63 bp, synthetic construct
  source 1..63: mol_type = other DNA
tgcaccattgggaatgcagtactgctggacacgggggtggctacctggacatcggcaatgtgg

SEQ ID NO: 6 - DNA, 75 bp, synthetic construct
  source 1..75: mol_type = other DNA
tgcaccattgggaatgcagtactgctgaacggaggggtggacacgggggtggctacctggacatcggcaatgtgg

SEQ ID NO: 7 - DNA, 38 bp, synthetic construct
  source 1..38: mol_type = other DNA
ttgggaatgcagtactgctggacacgggggtggctacc

SEQ ID NO: 8 - DNA, 43 bp, synthetic construct
  source 1..43: mol_type = other DNA
ttgggaatgcagtactgcaggggtggacacgggggtggctacc

SEQ ID NO: 9 - DNA, 37 bp, synthetic construct
  source 1..37: mol_type = other DNA
ttgggaatgcggaggggtggacacgggggtggctacc

SEQ ID NO: 10 - DNA, 43 bp, synthetic construct
  source 1..43: mol_type = other DNA
ttgggaatgcagtactggaggggtggacacgggggtggctacc

SEQ ID NO: 11 - DNA, 49 bp, synthetic construct
  source 1..49: mol_type = other DNA
ttgggaatgcagtactgctgaaggaggggtggacacgggggtggctacc

SEQ ID NO: 12 - DNA, 23 bp, synthetic construct
  source 1..23: mol_type = other DNA
acctccgagaggacccctccagg

SEQ ID NO: 13 - DNA, 23 bp, synthetic construct
  source 1..23: mol_type = other DNA
atttagctctactaagcgagagg

SEQ ID NO: 14 - DNA, 23 bp, synthetic construct
  source 1..23: mol_type = other DNA
ggatgattcgtgactgcttgagg

SEQ ID NO: 15 - DNA, 25 bp, synthetic construct
  source 1..25: mol_type = other DNA
acgtcaaatagctcggtttggatgg

SEQ ID NO: 16 - DNA, 23 bp, synthetic construct
  source 1..23: mol_type = other DNA
ggggaaaggagccactttagagg

SEQ ID NO: 17 - DNA, 23 bp, synthetic construct
  source 1..23: mol_type = other DNA
ggtatcataaggatccatctagg

SEQ ID NO: 18 - DNA, 58 bp, synthetic construct
  source 1..58: mol_type = other DNA
tcctcaccttttaaatatattttatctttagggcttaggtgtgatcattgcaattttg

SEQ ID NO: 19 - DNA, 58 bp, synthetic construct
  source 1..58: mol_type = other DNA
tcctcaccttttaaatatattttatctttagggcttaggtgtgatcgttgcaattttg

SEQ ID NO: 20 - DNA, 58 bp, synthetic construct; identical to SEQ ID NO: 19
  source 1..58: mol_type = other DNA

SEQ ID NO: 21 - RNA, 102 nt, synthetic construct
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 22 - protein, 7 aa, synthetic construct
PKKKRKV

SEQ ID NO: 23 - protein, 7 aa, synthetic construct
PKKKRRV

SEQ ID NO: 24 - protein, 16 aa, synthetic construct
KRPAATKKAGQAKKKK

SEQ ID NO: 25 - DNA, 60 bp, synthetic construct
  source 1..60: mol_type = other DNA
tagcaataaaggatcgtttattttcattggaagcgtgtgttggttttttgatcaggcgcg

SEQ ID NO: 26 - DNA, 106 bp, synthetic construct
  misc_feature 1..24: note = This region may encompass 16-24 nucleotides
  source 1..106: mol_type = other DNA
nnnnnnnnnnnnnnnnnnnnnnnngttttagtactctggaaacagaatctactaaaacaaggcaaaatgccgtgtttatctcgtcaacttgttggcgagatttttt

SEQ ID NO: 27 - RNA, 106 nt, synthetic construct
  misc_feature 1..24: note = This region may encompass 16-24 nucleotides
  source 1..106: mol_type = other RNA
nnnnnnnnnnnnnnnnnnnnnnnngttttagtactctggaaacagaatctactaaaacaaggcaaaatgccgtgtttatctcgtcaacttgttggcgagatttttt

SEQ ID NO: 28 - DNA, 106 bp, synthetic construct
  misc_feature 1..24: note = This region may encompass 16-24 nucleotides
  source 1..106: mol_type = other DNA
nnnnnnnnnnnnnnnnnnnnnnnngttatagtactctggaaacagaatctactataacaaggcaaaatgccgtgtttatctcgtcaacttgttggcgagatttttt

SEQ ID NO: 29 - RNA, 106 nt, synthetic construct
  misc_feature 1..24: note = This region may encompass 16-24 nucleotides
  source 1..106: mol_type = other RNA
nnnnnnnnnnnnnnnnnnnnnnnngttatagtactctggaaacagaatctactataacaaggcaaaatgccgtgtttatctcgtcaacttgttggcgagatttttt

SEQ ID NO: 30 - DNA, 20 bp, synthetic construct
  source 1..20: mol_type = other DNA
gagagcatgatttatattaa

SEQ ID NO: 31 - DNA, 20 bp, synthetic construct
  source 1..20: mol_type = other DNA
ggccccctatggcattgctt

SEQ ID NO: 32 - DNA, 20 bp, synthetic construct
  source 1..20: mol_type = other DNA
gtccttttttattttcctta

SEQ ID NO: 33 - DNA, 20 bp, synthetic construct
  source 1..20: mol_type = other DNA
gtgatggatacattaattag

SEQ ID NO: 34 - DNA, 20 bp, synthetic construct
  source 1..20: mol_type = other DNA
ggatatttggaaactatcta

SEQ ID NO: 35 - DNA, 20 bp, synthetic construct
  source 1..20: mol_type = other DNA
gcatatcatttaatttcaat

SEQ ID NO: 36 - DNA, 20 bp, synthetic construct
  source 1..20: mol_type = other DNA
gtagaaaagcatttcctaaa

SEQ ID NO: 37 - DNA, 20 bp, synthetic construct
  source 1..20: mol_type = other DNA
gtcagctgataactattttt

SEQ ID NO: 38 - DNA, 20 bp, synthetic construct
  source 1..20: mol_type = other DNA
gtacttatcatgtttttggg

SEQ ID NO: 39 - DNA, 21 bp, synthetic construct
  source 1..21: mol_type = other DNA
gaaaagcatttcctaaactgg

SEQ ID NO: 40 - DNA, 21 bp, synthetic construct
  source 1..21: mol_type = other DNA
gcatatcatttaatttcaata

SEQ ID NO: 41 - DNA, 21 bp, synthetic construct
  source 1..21: mol_type = other DNA
ggagagcatgatttatattaa

SEQ ID NO: 42 - DNA, 21 bp, synthetic construct
  source 1..21: mol_type = other DNA
gaacttattcattctgtctag

SEQ ID NO: 43 - DNA, 21 bp, synthetic construct
  source 1..21: mol_type = other DNA
gttatttaaattcatggatat

SEQ ID NO: 44 - DNA, 21 bp, synthetic construct
  source 1..21: mol_type = other DNA
ggcatatcatttaatttcaat

SEQ ID NO: 45 - DNA, 21 bp, synthetic construct
  source 1..21: mol_type = other DNA
gccccctatggcattgcttgt

SEQ ID NO: 46 - DNA, 21 bp, synthetic construct
  source 1..21: mol_type = other DNA
ggtccttttttattttcctta

SEQ ID NO: 47 - DNA, 21 bp, synthetic construct
  source 1..21: mol_type = other DNA
ggtgatggatacattaattag

SEQ ID NO: 48 - DNA, 22 bp, synthetic construct
  source 1..22: mol_type = other DNA
gttatttaaattcatggatatt

SEQ ID NO: 49 - DNA, 22 bp, synthetic construct
  source 1..22: mol_type = other DNA
gtttgtgtctcgtctatcttga

SEQ ID NO: 50 - DNA, 22 bp, synthetic construct
  source 1..22: mol_type = other DNA
gataaaagggtattacaaggca

SEQ ID NO: 51 - DNA, 22 bp, synthetic construct
  source 1..22: mol_type = other DNA
ggccccctatggcattgcttgt

SEQ ID NO: 52 - DNA, 22 bp, synthetic construct
  source 1..22: mol_type = other DNA
ggcatatcatttaatttcaata

SEQ ID NO: 53 - DNA, 22 bp, synthetic construct
  source 1..22: mol_type = other DNA
gaaaagcatttcctaaactgga

SEQ ID NO: 54 - DNA, 20 bp, synthetic construct
  source 1..20: mol_type = other DNA
gaatttaaataactcactgc

SEQ ID NO: 55 - DNA, 20 bp, synthetic construct
  source 1..20: mol_type = other DNA
gcttttctacatatgagttc

SEQ ID NO: 56 - DNA, 20 bp, synthetic construct
  source 1..20: mol_type = other DNA
gatgatacgaacacaaaata

SEQ ID NO: 57 - DNA, 20 bp, synthetic construct
  source 1..20: mol_type = other DNA
ggattaaaccaaaaattgcc

SEQ ID NO: 58 - DNA, 20 bp, synthetic construct
  source 1..20: mol_type = other DNA
ggagtacacatatacatttt

SEQ ID NO: 59 - DNA, 20 bp, synthetic construct
  source 1..20: mol_type = other DNA
gttaaagaacttgccttcat

SEQ ID NO: 60 - DNA, 20 bp, synthetic construct
  source 1..20: mol_type = other DNA
gtgctcatttaaaattatag

SEQ ID NO: 61 - DNA, 20 bp, synthetic construct
  source 1..20: mol_type = other DNA
gcagtgagttatttaaattc

SEQ ID NO: 62 - DNA, 21 bp, synthetic construct
  source 1..21: mol_type = other DNA
gatgtttaataaaaggttaag

SEQ ID NO: 63 - DNA, 21 bp, synthetic construct
  source 1..21: mol_type = other DNA
gtgatggatacattaattagc

SEQ ID NO: 64 - DNA, 21 bp, synthetic construct
  source 1..21: mol_type = other DNA
gataaaatatatttaaaaggt

SEQ ID NO: 65 - DNA, 22 bp, synthetic construct
  source 1..22: mol_type = other DNA
gtaaggttattctaaaagataa

SEQ ID NO: 66 - DNA, 22 bp, synthetic construct
  source 1..22: mol_type = other DNA
gaacttgccttcattggagttc

SEQ ID NO: 67 - DNA, 22 bp, synthetic construct
  source 1..22: mol_type = other DNA
ggtgatggatacattaattagc

SEQ ID NO: 68 - DNA, 22 bp, synthetic construct
  source 1..22: mol_type = other DNA
gaaattaaatgatatgccttag

SEQ ID NO: 69 - DNA, 22 bp, synthetic construct
  source 1..22: mol_type = other DNA
gaaggcaagttctttaacagtg

SEQ ID NO: 70 - DNA, 22 bp, synthetic construct
  source 1..22: mol_type = other DNA
gataaaatagaggagcatacaa

SEQ ID NO: 71 - DNA, 20 bp, synthetic construct
  source 1..20: mol_type = other DNA
gtccccttctgagagagaga

SEQ ID NO: 72 - DNA, 20 bp, synthetic construct
  source 1..20: mol_type = other DNA
ggtctatccctctcccaatt

SEQ ID NO: 73 - DNA, 20 bp, synthetic construct
  source 1..20: mol_type = other DNA
ggggtctatccctctcccaa

SEQ ID NO: 74 - DNA, 20 bp, synthetic construct
  source 1..20: mol_type = other DNA
gaaagataagtttgtatata

SEQ ID NO: 75 - DNA, 20 bp, synthetic construct
  source 1..20: mol_type = other DNA
gccatatacccatgtagaga

SEQ ID NO: 76 - DNA, 20 bp, synthetic construct
  source 1..20: mol_type = other DNA
gaggtaaagtccccttctga

SEQ ID NO: 77 - DNA, 20 bp, synthetic construct
  source 1..20: mol_type = other DNA
ggtggccatatacccatgta

SEQ ID NO: 78 - DNA, 20 bp, synthetic construct
  source 1..20: mol_type = other DNA
ggaccactgcagtcaggact

SEQ ID NO: 79 - DNA, 20 bp, synthetic construct
  source 1..20: mol_type = other DNA
gaagccacaaaccagaaaca

SEQ ID NO: 80 - DNA, 20 bp, synthetic construct
  source 1..20: mol_type = other DNA
gaagttacctaagttaacaa

SEQ ID NO: 81 - DNA, 20 bp, synthetic construct
  source 1..20: mol_type = other DNA
gtagaaattgagtctcaatt

SEQ ID NO: 82 - DNA, 20 bp, synthetic construct
  source 1..20: mol_type = other DNA
gtaatcagtgtcaaccaggt

SEQ ID NO: 83 - DNA, 20 bp, synthetic construct
  source 1..20: mol_type = other DNA
gtggtaagtatcccagaaga

SEQ ID NO: 84 - DNA, 20 bp, synthetic construct
  source 1..20: mol_type = other DNA
gagaggtaaagtccccttct

SEQ ID NO: 85 - DNA, 20 bp, synthetic construct
  source 1..20: mol_type = other DNA
gtagaattataaacaatttc

SEQ ID NO: 86 - DNA, 20 bp, synthetic construct
  source 1..20: mol_type = other DNA
ggtaaagtccccttctgaga

SEQ ID NO: 87 - DNA, 20 bp, synthetic construct
  source 1..20: mol_type = other DNA
gggtctatccctctcccaat

SEQ ID NO: 88 - DNA, 20 bp, synthetic construct
  source 1..20: mol_type = other DNA
gagagaggtaaagtcccctt

SEQ ID NO: 89 - DNA, 21 bp, synthetic construct
  source 1..21: mol_type = other DNA
gtaaagtccccttctgagaga

SEQ ID NO: 90 - DNA, 21 bp, synthetic construct
  source 1..21: mol_type = other DNA
gggtctatccctctcccaatt

SEQ ID NO: 91 - DNA, 21 bp, synthetic construct
  source 1..21: mol_type = other DNA
gggtggccatatacccatgta

SEQ ID NO: 92 - DNA, 21 bp, synthetic construct
  source 1..21: mol_type = other DNA
gctatatgttctagttttata

SEQ ID NO: 93 - DNA, 21 bp, synthetic construct
  source 1..21: mol_type = other DNA
gatataaatacagtaatgatt

SEQ ID NO: 94 - DNA, 21 bp, synthetic construct
  source 1..21: mol_type = other DNA
gagacaaatgaatatgtatca

SEQ ID NO: 95 - DNA, 21 bp, synthetic construct
  source 1..21: mol_type = other DNA
ggccatatacccatgtagaga

SEQ ID NO: 96 - DNA, 21 bp, synthetic construct
  source 1..21: mol_type = other DNA
gagtcaattcgaattttcctc

SEQ ID NO: 97 - DNA, 21 bp, synthetic construct
  source 1..21: mol_type = other DNA
ggtggtaagtatcccagaaga

SEQ ID NO: 98 - DNA, 21 bp, synthetic construct
  source 1..21: mol_type = other DNA
gtagaagccacaaaccagaaa

SEQ ID NO: 99 - DNA, 21 bp, synthetic construct
  source 1..21: mol_type = other DNA
ggtagaattataaacaatttc

SEQ ID NO: 100 - DNA, 21 bp, synthetic construct
  source 1..21: mol_type = other DNA
gtcaattttattttccagaga

SEQ ID NO: 101 - DNA, 21 bp, synthetic construct
  source 1..21: mol_type = other DNA
ggaaagataagtttgtatata

SEQ ID NO: 102 - DNA, 21 bp, synthetic construct
  source 1..21: mol_type = other DNA
ggggtctatccctctcccaat

SEQ ID NO: 103 - DNA, 21 bp, synthetic construct
  source 1..21: mol_type = other DNA
gtgtaagatcaaagagacaag

SEQ ID NO: 104 - DNA, 22 bp, synthetic construct
  source 1..22: mol_type = other DNA
gtccccttctgagagagagaga

SEQ ID NO: 105 - DNA, 22 bp, synthetic construct
  source 1..22: mol_type = other DNA
gatataaatacagtaatgattt

SEQ ID NO: 106 - DNA, 22 bp, synthetic construct
  source 1..22: mol_type = other DNA
ggggtctatccctctcccaatt

SEQ ID NO: 107 - DNA, 22 bp, synthetic construct
  source 1..22: mol_type = other DNA
ggctatatgttctagttttata

SEQ ID NO: 108 - DNA, 22 bp, synthetic construct
  source 1..22: mol_type = other DNA
gtgtagaaattgagtctcaatt

SEQ ID NO: 109 - DNA, 22 bp, synthetic construct
  source 1..22: mol_type = other DNA
gagagaggtaaagtccccttct

SEQ ID NO: 110 - DNA, 22 bp, synthetic construct
  source 1..22: mol_type = other DNA
ggtaaagtccccttctgagaga

SEQ ID NO: 111 - DNA, 22 bp, synthetic construct
  source 1..22: mol_type = other DNA
gccgatcggatttatttcataa

SEQ ID NO: 112 - DNA, 22 bp, synthetic construct
  source 1..22: mol_type = other DNA
gatttttaaaaaactgttaaaa

SEQ ID NO: 113 - DNA, 22 bp, synthetic construct
  source 1..22: mol_type = other DNA
gccatatacccatgtagagaaa

SEQ ID NO: 114 - DNA, 22 bp, synthetic construct
  source 1..22: mol_type = other DNA
gagaagttacctaagttaacaa

SEQ ID NO: 115 - DNA, 22 bp, synthetic construct
  source 1..22: mol_type = other DNA
gtgtaatcagtgtcaaccaggt

SEQ ID NO: 116 - DNA, 22 bp, synthetic construct
  source 1..22: mol_type = other DNA
gtcaattttattttccagagaa

SEQ ID NO: 117 - DNA, 22 bp, synthetic construct
  source 1..22: mol_type = other DNA
gagaggtaaagtccccttctga

SEQ ID NO: 118 - DNA, 22 bp, synthetic construct
  source 1..22: mol_type = other DNA
gaggtaaagtccccttctgaga

SEQ ID NO: 119 - DNA, 22 bp, synthetic construct
  source 1..22: mol_type = other DNA
gtatatggccaccctatgtccc

SEQ ID NO: 120 - DNA, 22 bp, synthetic construct
  source 1..22: mol_type = other DNA
gtctccataatcttcctgtctt

SEQ ID NO: 121 - DNA, 20 bp, synthetic construct
  source 1..20: mol_type = other DNA
gttaaatagttatatatgtg

SEQ ID NO: 122 - DNA, 20 bp, synthetic construct
  source 1..20: mol_type = other DNA
gaccactgcagtcaggactc

SEQ ID NO: 123 - DNA, 20 bp, synthetic construct
  source 1..20: mol_type = other DNA
gtaagatcaaagagacaaga

SEQ ID NO: 124 - DNA, 20 bp, synthetic construct
  source 1..20: mol_type = other DNA
gggtgtcacgtacttataaa

SEQ ID NO: 125 - DNA, 20 bp, synthetic construct
  source 1..20: mol_type = other DNA
gagacaagaaggaattgatg

SEQ ID NO: 126 - DNA, 21 bp, synthetic construct
  source 1..21: mol_type = other DNA
gatttacttcaagtgtagaaa

SEQ ID NO: 127 - DNA, 21 bp, synthetic construct
  source 1..21: mol_type = other DNA
gtccttctccttaggttttta

SEQ ID NO: 128 - DNA, 21 bp, synthetic construct
  source 1..21: mol_type = other DNA
gcatggcccaattatcctagg

SEQ ID NO: 129 - DNA, 21 bp, synthetic construct
  source 1..21: mol_type = other DNA
gtgtgatttgcttgccagaga

SEQ ID NO: 130 - DNA, 21 bp, synthetic construct
  source 1..21: mol_type = other DNA
gaagttacctaagttaacaaa

SEQ ID NO: 131 - DNA, 21 bp, synthetic construct
  source 1..21: mol_type = other DNA
ggaccactgcagtcaggactc

SEQ ID NO: 132 - DNA, 21 bp, synthetic construct
  source 1..21: mol_type = other DNA
ggggtgtcacgtacttataaa

SEQ ID NO: 133 - DNA, 21 bp, synthetic construct
  source 1..21: mol_type = other DNA
gttaattgattgcaaatttga

SEQ ID NO: 134 - DNA, 21 bp, synthetic construct
  source 1..21: mol_type = other DNA
gaggtaaccaaccaaacaaaa

SEQ ID NO: 135 - DNA, 22 bp, synthetic construct
  source 1..22: mol_type = other DNA
gtgtaagatcaaagagacaaga

SEQ ID NO: 136 - DNA, 22 bp, synthetic construct
  source 1..22: mol_type = other DNA
ggtccttctccttaggttttta

SEQ ID NO: 137 - DNA, 22 bp, synthetic construct
  source 1..22: mol_type = other DNA
gttataatttctagaggaaaat

SEQ ID NO: 138 - DNA, 22 bp, synthetic construct
  source 1..22: mol_type = other DNA
gtacgtgacacccctggccaca

SEQ ID NO: 139 - DNA, 22 bp, synthetic construct
  source 1..22: mol_type = other DNA
gttttttaaaaatcagatcaac

SEQ ID NO: 140 - DNA, 22 bp, synthetic construct
  source 1..22: mol_type = other DNA
gtcctgtcagccaatattagct

SEQ ID NO: 141 - DNA, 22 bp, synthetic construct
  source 1..22: mol_type = other DNA
gccaatattagctctgagttat

SEQ ID NO: 142 - DNA, 22 bp, synthetic construct
  source 1..22: mol_type = other DNA
gattaacctgaaggtaaaaaga

SEQ ID NO: 143 - DNA, 22 bp, synthetic construct
  source 1..22: mol_type = other DNA
tattttatctttagggcttagg

SEQ ID NO: 144 - DNA, 22 bp, synthetic construct
  source 1..22: mol_type = other DNA
aaatatattttatctttagggc

SEQ ID NO: 145 - DNA, 22 bp, synthetic construct
  source 1..22: mol_type = other DNA
taaatatattttatctttaggg

SEQ ID NO: 146 - DNA, 22 bp, synthetic construct
  source 1..22: mol_type = other DNA
ccttttaaatatattttatctt

SEQ ID NO: 147 - DNA, 22 bp, synthetic construct
  source 1..22: mol_type = other DNA
accttttaaatatattttatct

SEQ ID NO: 148 - DNA, 22 bp, synthetic construct
  source 1..22: mol_type = other DNA
caccttttaaatatattttatc

SEQ ID NO: 149 - DNA, 22 bp, synthetic construct
  source 1..22: mol_type = other DNA
gatcacacctaagccctaaaga

SEQ ID NO: 150 - DNA, 22 bp, synthetic construct
  source 1..22: mol_type = other DNA
tgatcacacctaagccctaaag

SEQ ID NO: 151 - DNA, 22 bp, synthetic construct
  source 1..22: mol_type = other DNA
atgatcacacctaagccctaaa

SEQ ID NO: 152 - DNA, 22 bp, synthetic construct
  source 1..22: mol_type = other DNA
gcaatgatcacacctaagccct

SEQ ID NO: 153 - DNA, 22 bp, synthetic construct
  source 1..22: mol_type = other DNA
tgcaatgatcacacctaagccc

SEQ ID NO: 154 - DNA, 22 bp, synthetic construct
  source 1..22: mol_type = other DNA
ttgcaatgatcacacctaagcc

SEQ ID NO: 155 - DNA, 22 bp, synthetic construct
  source 1..22: mol_type = other DNA
attgcaatgatcacacctaagc

SEQ ID NO: 156 - DNA, 22 bp, synthetic construct
  source 1..22: mol_type = other DNA
ccttttaaatatattttatctt

SEQ ID NO: 157 - DNA, 21 bp, synthetic construct
  source 1..21: mol_type = other DNA
attttatctttagggcttagg

SEQ ID NO: 158 - DNA, 21 bp, synthetic construct
  source 1..21: mol_type = other DNA
aatatattttatctttagggc

SEQ ID NO: 159 - DNA, 21 bp, synthetic construct
  source 1..21: mol_type = other DNA
aaatatattttatctttaggg

SEQ ID NO: 160 - DNA, 21 bp, synthetic construct
  source 1..21: mol_type = other DNA
cttttaaatatattttatctt

SEQ ID NO: 161 - DNA, 21 bp, synthetic construct
  source 1..21: mol_type = other DNA
ccttttaaatatattttatct

SEQ ID NO: 162 - DNA, 21 bp, synthetic construct
  source 1..21: mol_type = other DNA
accttttaaatatattttatc

SEQ ID NO: 163 - DNA, 21 bp, synthetic construct
  source 1..21: mol_type = other DNA
atcacacctaagccctaaaga

SEQ ID NO: 164 - DNA, 21 bp, synthetic construct
  source 1..21: mol_type = other DNA
gatcacacctaagccctaaag

SEQ ID NO: 165 - DNA, 21 bp, synthetic construct
  source 1..21: mol_type = other DNA
tgatcacacctaagccctaaa

SEQ ID NO: 166 - DNA, 21 bp, synthetic construct
  source 1..21: mol_type = other DNA
caatgatcacacctaagccct

SEQ ID NO: 167 - DNA, 21 bp, synthetic construct
  source 1..21: mol_type = other DNA
gcaatgatcacacctaagccc

SEQ ID NO: 168 - DNA, 21 bp, synthetic construct
  source 1..21: mol_type = other DNA
tgcaatgatcacacctaagcc

SEQ ID NO: 169 - DNA, 21 bp, synthetic construct
  source 1..21: mol_type = other DNA
ttgcaatgatcacacctaagc

SEQ ID NO: 170 - DNA, 21 bp, synthetic construct
  source 1..21: mol_type = other DNA
cttttaaatatattttatctt

SEQ ID NO: 171 - DNA, 20 bp, synthetic construct
  source 1..20: mol_type = other DNA
ttttatctttagggcttagg

SEQ ID NO: 172 - DNA, 20 bp, synthetic construct
  source 1..20: mol_type = other DNA
atatattttatctttagggc

SEQ ID NO: 173 - DNA, 20 bp, synthetic construct
  source 1..20: mol_type = other DNA
aatatattttatctttaggg

SEQ ID NO: 174 - DNA, 20 bp, synthetic construct
  source 1..20: mol_type = other DNA
ttttaaatatattttatctt

SEQ ID NO: 175 - DNA, 20 bp, synthetic construct
  source 1..20: mol_type = other DNA
cttttaaatatattttatct

SEQ ID NO: 176 - DNA, 20 bp, synthetic construct
  source 1..20: mol_type = other DNA
ccttttaaatatattttatc

SEQ ID NO: 177 - DNA, 20 bp, synthetic construct
  source 1..20: mol_type = other DNA
tcacacctaagccctaaaga

SEQ ID NO: 178 - DNA, 20 bp, synthetic construct
  source 1..20: mol_type = other DNA
atcacacctaagccctaaag

SEQ ID NO: 179 - DNA, 20 bp, synthetic construct
  source 1..20: mol_type = other DNA
gatcacacctaagccctaaa

SEQ ID NO: 180 - DNA, 20 bp, synthetic construct
  source 1..20: mol_type = other DNA
aatgatcacacctaagccct

SEQ ID NO: 181 - DNA, 20 bp, synthetic construct
  source 1..20: mol_type = other DNA
caatgatcacacctaagccc

SEQ ID NO: 182 - DNA, 20 bp, synthetic construct
  source 1..20: mol_type = other DNA
gcaatgatcacacctaagcc

SEQ ID NO: 183 - DNA, 20 bp, synthetic construct
  source 1..20: mol_type = other DNA
tgcaatgatcacacctaagc

SEQ ID NO: 184 - DNA, 20 bp, synthetic construct
  source 1..20: mol_type = other DNA
ttttaaatatattttatctt

SEQ ID NO: 185 - DNA, 20 bp, synthetic construct
  source 1..20: mol_type = other DNA
ttttatctttagggcttagg

SEQ ID NO: 186 - DNA, 20 bp, synthetic construct
  source 1..20: mol_type = other DNA
aatatattttatctttaggg

SEQ ID NO: 187 - DNA, 20 bp, synthetic construct
  source 1..20: mol_type = other DNA
aaattgcaatgatcacacct

SEQ ID NO: 188 - DNA, 264 bp, synthetic construct
  source 1..264: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 189 - DNA, 40 bp, Mus sp.
  source 1..40: mol_type = genomic DNA
attgggaatgcagtactgctgaacggaggggtggacacgg